(19) United States
(12) Patent Application Publication
(10) Pub. No.: US 2024/0382605 A1
(43) Pub. Date: Nov. 21, 2024

(54) CONJUGATED OLIGONUCLEOTIDES AND USES THEREOF

(71) Applicants: Ionis Pharmaceuticals, Inc., Carlsbad, CA (US); BicycleTx Limited, Cambridge (GB)

(72) Inventors: Punit P. Seth, Carlsbad, CA (US); Michael Oestergaard, Carlsbad, CA (US); Michele Carrer, San Marcos, CA (US); Michael Tanowitz, Solana Beach, CA (US); Michael Rigby, Cambridge (GB); Michael Skynner, Cambridge (GB); Steven Stanway, Cambridge (GB); Liudvikas Urbonas, Cambridge (GB); Katerine Van Rietschoten, Cambridge (GB)

(73) Assignees: Ionis Pharmaceuticals, Inc., Carlsbad, CA (US); BicycleTx Limited, Cambridge (GB)

(21) Appl. No.: 18/696,199

(22) PCT Filed: Sep. 29, 2022
(86) PCT No.: PCT/US2022/077301

§ 371 Date: Mar. 27, 2024

Related U.S. Application Data
(60) Provisional application No. 63/340,130, filed on May 10, 2022.
(60) Provisional application No. 63/250,135, filed on Sep. 29, 2021.

(51) Int. Cl. A61K 47/64; A61K 9/00; A61P 21/00; C12N 15/113

(52) U.S. Cl. CPC A61K 47/64; A61K 9/0019; A61P 21/00; C12N 15/1137; C12N 2310/14; C12N 2310/314; C12N 2310/315; C12N 2310/321; C12N 2310/322; C12N 2320/33; C12Y 207/11

(57) ABSTRACT

Provided herein are oligomeric compounds comprising a bicycle ligand and a modified oligonucleotide. This compound may comprise a bicycle ligand as the cell-targeting moiety, and may also comprise a conjugate linker to connect the bicycle ligand and modified oligonucleotide. This compound may be used in conjunction with a pharmaceutically acceptable salt.

### SEQUENCE LISTING

The present application is being filed along with a Sequence Listing in electronic format. The Sequence Listing is provided as a file entitled CHEM0103WOSEQ.xml created Sep. 29, 2022, which is 3,314 kb in size. The information in the electronic format of the sequence listing is incorporated herein by reference in its entirety.

### FIELD

The present embodiments provide compounds and methods for targeting cells of interest with an oligonucleotide.

### BACKGROUND

Oligomeric compounds, such as siRNA and single-stranded antisense oligonucleotides (ASOs), have been shown to be useful for regulating gene expression and have proven to be therapeutically effective. Certain chemical modifications of oligomeric compounds can improve the potency, efficacy, and unwanted side effects of oligomeric compounds, allowing for administration of lower doses, reducing the potential for toxicity, and decreasing the overall cost of therapy. Oligomeric compounds can be modified with a conjugate group, e.g., a ligand for a receptor expressed on a cell of interest, which results in targeting the oligomeric compound to one or more tissues of interest.

The type 1 transferrin receptor (TfR1), also known as CD71, is a transmembrane glycoprotein that binds to and internalizes iron-bound transferrin by receptor-mediated endocytosis. Antibody-drug conjugates with an anti-TfR1 antibody have been used to deliver drugs to various tissues, including the CNS and muscle.

Cyclic peptides are able to bind with high affinity and specificity to protein targets and hence are an attractive molecule class for the development of therapeutics. In fact, several cyclic peptides are already successfully used in the clinic, as for example the antibacterial peptide vancomycin, the immunosuppressant drug cyclosporine, or the anti-cancer drug octreotide (Driggers et al. (2008), Nat. Rev. Drug. Discov. 7(7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å2; Wu et al. (2007), Science 330, 1066-71), a cyclic peptide with the Arg-Gly-Asp motif binding to integrin αVb3 (355 Å2) (Xiong et al. (2002), Science 296(5565), 151-5) or the cyclic peptide inhibitor upain-1 binding to urokinase-type plasminogen activator (603 Å2; Zhao et al. (2007), J. Struct. Biol. 160(1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Chemey et al. (1998), J. Med. Chem. 41(11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers utilized tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example 1,1′,1″-(1,3,5-triazinane-1,3,5-triyl)triprop-2-en-1-one (TATA) (Heinis et al. (2014) Angewandte Chemie, International Edition 53(6) 1602-1606).

Phage display-based combinatorial approaches have been developed to generate and screen large libraries of bicyclic peptides to targets of interest (Heinis et al. (2009), Nat. Chem. Biol. 5(7), 502-7 and WO 2009/098450). Briefly, combinatorial libraries of linear peptides containing three cysteine residues and two regions of six random amino acids (Cys-(Xaa)6-Cys-(Xaa)6-Cys) were displayed on phage and cyclized by covalently linking the cysteine side chains to a small molecule scaffold.

### SUMMARY

Embodiments provided herein are directed to oligomeric compounds that comprise an oligonucleotide and a conjugate group. In certain embodiments, the conjugate group comprises a cell-targeting moiety. In certain embodiments, the conjugate group comprises a conjugate linker that links the cell-targeting moiety to the oligonucleotide. In certain embodiments, the cell-targeting moiety comprises or consists of a polypeptide comprising two polypeptide loops attached to a molecular scaffold. In certain embodiments, the cell-targeting moiety comprises or consists of a bicycle ligand. In certain embodiments, the conjugate linker links the oligonucleotide to the polypeptide. In general, conjugate linkers have sufficient length and/or structure to separate the oligonucleotide and the bicycle ligand such that the bicycle ligand does not inhibit an activity of the oligonucleotide (e.g., antisense activity) and the oligonucleotide does not inhibit an activity of the bicycle ligand (e.g., binding the transferrin receptor).

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank, NCBI, and ENSEMBL reference sequence records are hereby expressly incorporated by reference for the portions of the document discussed herein, as well as in their entirety.

It is understood that the sequence set forth in each SEQ ID NO of an oligonucleotide in the examples contained herein is independent of any modification to a sugar moiety, an internucleoside linkage, or a nucleobase. As such, oligonucleotides defined by a SEQ ID NO may comprise, independently, one or more modifications to a sugar moiety, an internucleoside linkage, or a nucleobase.

It is understood that throughout the specification, the first letter in a peptide sequence is the first amino acid of the peptide at the N-terminus and the last letter in a peptide sequence is the last amino acid of the peptide at the C-terminus unless indicated otherwise.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH₂CH₂OCH₃ group in place of the 2′—OH group of a furanosyl sugar moiety. A “2′-MOE sugar moiety” means a sugar moiety with a 2′-OCH₂CH₂OCH₃ group in place of the 2′—OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” means a nucleoside comprising a 2′-MOE sugar moiety.

As used herein, “2′-OMe” means a 2′-OCH₃ group in place of the 2′—OH group of a furanosyl sugar moiety. A “2′-O-methyl sugar moiety” or “2′-OMe sugar moiety” means a sugar moiety with a 2′-OCH₃ group in place of the 2′—OH group of a furanosyl sugar moiety. Unless otherwise indicated, a 2′-OMe sugar moiety is in the β-D-ribosyl configuration.

As used herein, “2′-OMe nucleoside” means a nucleoside comprising a 2′-OMe sugar moiety.

As used herein, “2′-F” means a 2′—F group in place of the 2′—OH group of a furanosyl sugar moiety. A “2′-fluoro sugar moiety” or “2′-F sugar moiety” means a sugar moiety with a 2′—F group in place of the 2′-OH group of a furanosyl sugar moiety. Unless otherwise indicated, a 2′-F sugar moiety is in the β-D-ribosyl configuration.

As used herein, “2′-F nucleoside” means a nucleoside comprising a 2′-F sugar moiety.

As used herein, “2′-NMA” means a —O—CH₂—C(═O)—NH—CH₃ group in place of the 2′—OH group of a ribosyl sugar moiety. A “2′-NMA sugar moiety” is a sugar moiety with a 2′-O—CH₂—C(═O)—NH—CH₃ group in place of the 2′—OH group of a ribosyl sugar moiety. Unless otherwise indicated, a 2′-NMA sugar moiety is in the β-D configuration. “NMA” means O—N-methyl acetamide.

As used herein, “2′-NMA nucleoside” means a nucleoside comprising a 2′-NMA sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5 position. A 5-methyl cytosine is a modified nucleobase.

As used herein, “administering” means providing an oligomeric agent or pharmaceutical composition to a subject.

As used herein, an “aliphatic amino acid” is an amino acid having a side chain composed of H and C. Aliphatic amino acids include, but are not limited to, glycine, alanine, leucine, isoleucine, valine, beta-alanine, 2-aminoisobutyric acid.

As used here in, an “amino acid” is a compound or a monomer subunit of a polypeptide having an amino group, a carboxylate group, and at least one carbon covalently linked between the amino group and carboxylate group that comprises an optional side chain. An “α-amino acid” contains exactly one carbon between the amino group and carboxylate group that bears an optional side chain. A “β-amino acid” contains exactly two optionally substituted carbons between the amino group and the carboxylate group.

As used here in, an “amino acid mimetic” is a compound or a monomer subunit of a peptidomimetic having an amino group surrogate, a carboxylate group surrogate, and at least one carbon covalently linked between the amino group surrogate and carboxylate group surrogate that comprises an optional side chain.

As used herein, an “aromatic amino acid” is an amino acid having an aromatic ring in its side chain. Aromatic amino acids include, but are not limited to, phenylalanine, tyrosine, and tryptophan.

As used herein, “bicycle ligand” means a ligand comprising a polypeptide or peptidomimetic covalently bound to a molecular scaffold at three distinct sites, forming two polypeptide loops. Typically, such peptides or peptidomimetics comprise a polypeptide having natural or non-natural amino acids or amino acid mimetics, including a first, a second, and a third amino acid comprising reactive groups that form covalent bonds to the scaffold. In certain embodiments, the peptides or peptidomimetics comprise at least three cysteine residues (referred to herein as Cᵢ, Cᵢᵢ and Cᵢᵢᵢ), which are the reactive groups. As used herein, “loop polypeptide” refers to the portion of the bicycle ligand that forms the two polypeptide loops, including the reactive groups, but excluding any N-terminal or C-terminal extensions. As used herein, “loop sequences” refers to the amino acids or amino acid mimetics between the reactive groups.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cell-targeting moiety” means a conjugate group or portion of a conjugate group that is capable of binding to a particular cell type or particular cell types. In certain embodiments, a cell-targeting moiety is capable of binding the cell-surface receptor or the cell-surface moiety. In certain embodiments, a cell-targeting moiety is capable of being internalized when it interacts with or binds the cell-surface receptor or the cell-surface moiety. In certain embodiments, a cell-targeting moiety comprises a bicyclic polypeptide or a bicycle ligand. In certain embodiments, a cell-targeting moiety consists of a bicyclic polypeptide or a bicycle ligand.

As used herein, “cell-surface moiety” means a moiety present on the surface of a cell that is available to interact with matter external to the cell. In certain embodiments, a portion of the cell-surface moiety is integral with the cell membrane of the cell. Non-limiting examples of cell-surface moieties are lipids, proteins, and carbohydrates. In certain embodiments, a cell-surface moiety is a cell-surface receptor. In certain embodiments, the cell-surface receptor is the transferrin receptor.

As used herein, “cell-surface receptor” means a protein receptor expressed on the surface of a cell that is available to interact with a corresponding ligand. The ligand may be endogenous or exogenous. In certain embodiments, the cell-surface receptor is the transferrin receptor.

As used herein, a “charged amino acid” is an amino acid having a side chain that comprises a positive charge or a negative charge when in solution at pH=7.0. A “basic amino acid” has a side chain that comprises a positive charge when in solution at pH=7.0. Basic amino acids include, but are not limited to, lysine, arginine, and ornithine. A “acidic amino acid” has a side chain that comprises a negative charge when in solution at pH=7.0. Acidic amino acids include, but are not limited to, glutamic acid and aspartic acid.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are oligomeric compounds disclosed herein. In certain embodiments, the oligomeric compounds are antisense compounds. In certain embodiments, the molecules are modified oligonucleotides. In certain embodiments, the molecules are oligomeric compounds comprising modified oligonucleotides.

As used herein, “cleavable moiety” means a bond or group of atoms that is cleaved under physiological conditions, for example, inside a cell, or a subject.

As used herein, “complementary” in reference to an oligonucleotide means that at least 70% of the nucleobases of the oligonucleotide and the nucleobases of another nucleic acid or one or more regions thereof are capable of hydrogen bonding with one another when the nucleobase sequence of the oligonucleotide and the other nucleic acid are aligned in opposing directions. “Complementary region” in reference to a region of an oligonucleotide means that at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions. Complementary nucleobases mean nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). Certain modified nucleobases that pair with natural nucleobases or with other modified nucleobases are known in the art and are not considered complementary nucleobases as defined herein unless indicated otherwise. For example, inosine can pair, but is not considered complementary, with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly attached to an oligonucleotide. In certain embodiments, a conjugate group comprises a conjugate moiety and a conjugate linker that attaches the conjugate moiety to the oligonucleotide. In certain embodiments, a conjugate group comprises a bicycle ligand.

As used herein, “conjugate linker” means a single bond or group of atoms comprising at least one bond that connects a conjugate moiety to an oligonucleotide.

As used herein, “conjugate moiety” means a group of atoms that is attached to an oligonucleotide via a conjugate linker. In certain embodiments, a conjugate moiety comprises a cell-targeting moiety. In certain embodiments, a cell-targeting moiety comprises or consists of a bicycle ligand. In certain embodiments, a cell-targeting moiety comprises or consists of a bicyclic polypeptide.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, a “cyclic amino acid” is an amino acid where the side chain connects to the backbone amide to form a cyclic structure. “Cyclic amino acid” includes, but is not limited to, proline or hydroxyproline.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif Unless otherwise indicated, the sugar moiety of each nucleoside of the gap is a 2′-β-D-deoxyribosyl sugar moiety. Thus, by way of example, the term “MOE gapmer” indicates a gapmer having a gap comprising 2′-β-D-deoxynucleosides and wings comprising 2′-MOE nucleosides. Unless otherwise indicated, a MOE gapmer may comprise one or more modified internucleoside linkages and/or modified nucleobases and such modifications do not necessarily follow the gapmer pattern of the sugar modifications.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “identical,” or “percent identical”, with regards to an amino acid sequence, means the percentage of amino acids that are identical between two amino acid sequences when the amino acid sequences are aligned for maximal similarity. “Identical” means that each atom of the amino acid is the same; that is, amino acids with substitutions or modifications do not count as “identical” amino acids.

As used herein, the term “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “non-bicyclic modified sugar moiety” means a modified sugar moiety that comprises a modification, such as a substituent, that does not form a bridge between two atoms of the sugar to form a second ring.

As used herein, “N-terminal modification” or “C-terminal modification” means a terminal non-peptidic chemical modification to a bicycle ligand on either side of the polypeptide, such as acylation or amidation, and that does not become part of a conjugate linker As used herein, “N-terminal extension” or “C-terminal extension” means a non-peptidic chemical modification to a bicycle ligand on either side of the polypeptide that is between terminus of the polypeptide of the bicycle ligand and the functional group that becomes part of the conjugate linker upon conjugation to an oligonucleotide.

As used herein, “mismatch” or “non-complementary” means a nucleobase of a first oligonucleotide that is not complementary with the corresponding nucleobase of a second oligonucleotide or target nucleic acid when the first and second oligonucleotide are aligned.

As used herein, “motif” means the pattern of unmodified and/or modified sugar moieties, nucleobases, and/or internucleoside linkages, in an oligonucleotide.

As used herein, “natural amino acid” means Gly or the _{L}-isomer of each of the following: Ala, Arg, Asn, Asp, Cys, Gln, Glu, His, Ile, Lys, Leu, Met, Phe, Pro, Ser, Thr, Trp, Tyr, Val.

As used herein, a “neutral amino acid” is an amino acid having a side chain that does not comprise a positive charge or a negative charge when in solution at pH=7.0. Neutral amino acids include, but are not limited to, 20 glycine, alanine, leucine, isoleucine, valine, serine, cysteine, methionine, proline, threonine, tyrosine, phenylalanine, tryptophan, beta-alanine, and 2-aminoisobutyric acid.

As used herein, “non-natural amino acid” means any amino acid other than the standard twenty amino acids encoded by the human genetic code, including _{D}-isomers of each of the following: Ala, Arg, Asn, Asp, Cys, Gln, Glu, His, Ile, Lys, Leu, Met, Phe, Pro, Ser, Thr, Trp, Tyr, Val.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one other nucleobase. A “5-methyl cytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound or fragment of a compound comprising a nucleobase and a sugar moiety. The nucleobase and sugar moiety are each, independently, unmodified or modified.

As used herein, “molecular scaffold” means a chemical group that forms covalent bonds with the reactive groups of a polypeptide to form at least two polypeptide loops joined by the molecular scaffold. In certain embodiments, the molecular scaffold is 1,1′,1″-(1,3,5-triazinane-1,3,5-triyl)triprop-2-en-1-one (TATA). In certain embodiments, the molecular scaffold is 1,1′,1″-(1,3,5-triazinane-1,3,5-triyl)tris(2-bromoethanone) (TATB).

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences.

As used herein, “oligonucleotide” means a strand of linked nucleosides connected via internucleoside linkages, wherein each nucleoside and internucleoside linkage independently may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “polypeptide” or “peptide” means a compound or a fragment of a compound consisting of 3 or more amino acids linked together via amide bonds. Unless otherwise indicated, polypeptides consist of 3-50 amino acids.

As used herein, “peptidomimetic” means a compound or a fragment of a compound consisting of 3 or more amino acids or amino acid mimetics linked together, wherein at least two subunits are linked by a bond that is not an amide bond. Unless otherwise indicated, peptide mimetics consist of 3-50 amino acids or amino acid mimetics.

As used herein, “pharmaceutically acceptable carrier or diluent” means any substance suitable for use in administering to a subject. Certain such carriers enable pharmaceutical compositions to be formulated as, for example, tablets, pills, dragees, capsules, liquids, gels, syrups, slurries, suspension, and lozenges for the oral ingestion by a subject. In certain embodiments, a pharmaceutically acceptable carrier or diluent is sterile water, distilled water for injection, sterile saline, sterile buffer solution or sterile artificial cerebrospinal fluid.

As used herein “pharmaceutically acceptable salt(s)” means physiologically and pharmaceutically acceptable salt(s) of oligomeric compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein “prodrug” means a therapeutic agent in a first form outside the body that is converted to a second form within a subject or cells thereof. Typically, conversion of a prodrug within the subject is facilitated by the action of an enzyme (e.g., endogenous or viral enzyme) or chemicals present in cells or tissues and/or by physiologic conditions. In certain embodiments, the first form of the prodrug is less active than the second form.

As used herein, “reactive group” means an atom or group of atoms of an amino acid that can form bonds with another compound, e.g., another atom or group of atoms of another amino acid or another compound. In certain embodiments, a reactive group is the sulfur atom of a cysteine amino acid.

As used herein, “self-complementary” in reference to an oligonucleotide means an oligonucleotide that at least partially hybridizes to itself.

As used herein, “side chain” has its ordinary meaning in the art and means a sub-structure of an amino acid that does not join the amino and carboxylate groups of an amino acid, and attaches to, e.g., the alpha or beta carbon of the amino acid.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Stabilized phosphate groups include, but are not limited to, 5′-vinyl phosphonates and 5′-cyclopropyl phosphonate.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the results of a synthetic method that is not designed to control the stereochemical configuration. In certain embodiments, a stereorandom chiral center is a stereorandom phosphorothioate internucleoside linkage.

As used herein, “standard cell assay” means assay(s) described in the Examples and reasonable variations thereof.

As used herein, “subject” refers to a human or non-human animal, including, but not limited to, mice, rats, rabbits, dogs, cats, pigs, and non-human primates, including, but not limited to, monkeys and chimpanzees.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “target nucleic acid” and “target RNA” mean a nucleic acid that an oligomeric compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “transferrin receptor”, “TfR1”, and “CD71” mean the mammalian type 1 transferrin receptor. “Human transferrin receptor” and “human TfR1” means the protein encoded by the gene represented by ENSEMBL ID ENSG00000072274 and/or GenBank Gene ID 7037. “Mouse transferrin 25 receptor” and “mouse TfR1” means the protein encoded by the gene represented by ENSEMBL ID ENSMUSG00000022797 and/or GenBank Gene ID 22042.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is the modulation of splicing of a target pre-mRNA.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount and/or activity, of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H agents may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “splice-modulating agent” means an antisense agent that acts, at least in part, by modulating the splicing of a target nucleic acid. A “splice-modulating” agent comprises a “splice-modulating oligonucleotide”.

As used herein, “steric-blocking agent” means an antisense agent that acts, at least in part, due to directly binding to a target nucleic acid, thus blocking the interaction of the target nucleic acid with other nucleic acids or proteins.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a oligomeric agent or pharmaceutical composition that provides a therapeutic benefit to a subject. For example, a therapeutically effective amount improves a symptom of a disease.

### CERTAIN EMBODIMENTS

The present disclosure provides the following non-limiting numbered embodiments:

Embodiment 1. An oligomeric compound comprising a modified oligonucleotide and a conjugate group, wherein the modified oligonucleotide consists of 10 to 300 linked nucleosides, and the conjugate group comprises a bicycle ligand and a conjugate linker, wherein
the bicycle ligand comprises a polypeptide consisting of 13-22 linked amino acids or amino acid mimetics and a molecular scaffold, wherein
   each of a first, a second, and a third amino acid of the polypeptide comprises a reactive group, each of which separately forms a bond with the molecular scaffold, thereby forming two polypeptide loops attached to the molecular scaffold; and
wherein a portion of the bicycle ligand binds to a type 1 transferrin receptor; and
wherein the modified oligonucleotide is covalently linked to the bicycle ligand through the conjugate linker.

Embodiment 2. The oligomeric compound of embodiment 1, wherein the conjugate group consists of the bicycle ligand and a conjugate linker.

Embodiment 3. The oligomeric compound of embodiment 1 or 2, wherein the oligomeric compound consists of the modified oligonucleotide and the conjugate group.

Embodiment 4. The oligomeric compound of any of embodiments 1-3, wherein the three reactive groups are each the thiol of a cysteine.

Embodiment 5. The oligomeric compound of any of embodiments 1-4, wherein the polypeptide has the following formula, from N-terminal to C-terminal:

[B]ₙ—[Zᵢ]-[J]ₘ—[Zᵢᵢ]—[O]ₒ—[Zᵢᵢᵢ]—[U]ₚ, wherein:
Zᵢ, Zᵢᵢ, and Zᵢᵢᵢ are the first, second, and third amino acids comprising a reactive group;
each B, J, O, and U is independently selected amino acids or amino acid mimetics;
n is from 0 to 5;
m is from 3 to 7;
o is from 3 to 7;
p is from 0 to 5;
wherein the sum of m+o is less than 12.

Embodiment 6. The oligomeric compound of embodiment 5, wherein m is 7 and o is 3.

Embodiment 7. The oligomeric compound of embodiment 5, wherein m is 2 and o is 9.

Embodiment 8. The oligomeric compound of embodiment 5, wherein m and o are both 6.

Embodiment 9. The oligomeric compound of embodiment 5, wherein m is 3 and o is 8.

Embodiment 10. The oligomeric compound of any of embodiments 5-9, wherein n is 0.

Embodiment 11. The oligomeric compound of any of embodiments 5-9, wherein n is 3 or 4.

Embodiment 12. The oligomeric compound of any of embodiments 5-11, wherein p is 0.

Embodiment 13. The oligomeric compound of any of embodiments 5-11, wherein p is 3 or 4.

Embodiment 14. The oligomeric compound of any of embodiments 1-13, wherein the polypeptide has an N-terminal modification.

Embodiment 15. The oligomeric compound of embodiment 14, wherein the N-terminal modification is an acetyl group.

Embodiment 16. The oligomeric compound of embodiment 14, wherein the N-terminal modification is an azidopropyl group.

Embodiment 17. The oligomeric compound of any of embodiments 1-13, wherein the polypeptide has a C-terminal modification.

Embodiment 18. The oligomeric compound of embodiment 14, wherein the C-terminal modification is an amide group.

Embodiment 19. The oligomeric compound of any of embodiments 1-18, wherein the conjugate linker is attached to the N-terminal amino acid of the bicycle ligand.

Embodiment 20. The oligomeric compound of any of embodiments 1-19, wherein the conjugate linker is attached to the C-terminal amino acid of the bicycle ligand.

Embodiment 21. The oligomeric compound of any of embodiments 1-20, wherein the conjugate linker is attached to a side chain of an amino acid within one of the polypeptide loops of the bicycle ligand.

Embodiment 22. The oligomeric compound of any of embodiments 1-21, wherein the bicycle ligand comprises a C-terminal extension.

Embodiment 23. The oligomeric compound of embodiment 22, wherein the C-terminal extension is selected from PEG10 or PEG24.

Embodiment 24. The oligomeric compound of any of embodiments 1-23, wherein the bicycle ligand comprises an N-terminal extension.

Embodiment 25. The oligomeric compound of embodiment 24, wherein the N-terminal extension is selected from PEG10 or PEG24.

Embodiment 26. The oligomeric compound of any of embodiments 1-25, wherein the conjugate group is attached to the 5′-terminal nucleoside of the modified oligonucleotide.

Embodiment 27. The oligomeric compound of embodiment 26, wherein the conjugate group is attached to the 5′-position of the 5′-terminal nucleoside of the modified oligonucleotide.

Embodiment 28. The oligomeric compound of any of embodiments 1-25, wherein the conjugate group is attached to the 3′-terminal nucleoside of the modified oligonucleotide.

Embodiment 29. The oligomeric compound of embodiment 28, wherein the conjugate group is attached to the 3′-position of the 3′-terminal nucleoside of the modified oligonucleotide.

Embodiment 30. The oligomeric compound of any of embodiments 1-25, wherein the conjugate group is attached to an internal nucleoside of the modified oligonucleotide.

Embodiment 31. The oligomeric compound of embodiment 1-25, wherein the conjugate group is attached through a modified internucleoside linkage.

Embodiment 32. The oligomeric compound of embodiment 28-30, wherein the conjugate group is attached through a 2′-modified furanosyl sugar moiety.

Embodiment 33. The oligomeric compound of any of embodiments 1-32, wherein the bicycle ligand has an amino acid sequence with at least 80% identity to any of SEQ ID NO: 26-27, 36-56, 58-65, 67-76, 79-88, 90-152, or 192-246.

Embodiment 34. The oligomeric compound of embodiment 33, wherein the bicycle ligand has an amino acid sequence with at least 85%, at least 90%, at least 95%, or 100% identity to any of SEQ ID NO: 26-27, 36-56, 58-65, 67-76, 79-88, 90-152, or 192-246.

Embodiment 35. The oligomeric compound of any of embodiments 5-34, wherein [Zᵢ]-[J]ₘ—[Zᵢᵢ]-[O]ₒ-[Zᵢᵢᵢ] has an amino acid sequence with at least 85%, at least 90%, at least 95%, or 100% identity to any of SEQ ID NO 26-27, 35-56, 58-65, 67-76, 79-88, 90-152, or 192-246.

Embodiment 36. The oligomeric compound of any of embodiments 5-35, wherein [Zᵢ]-[J]ₘ—[Zᵢ]-[ ]o-[Zᵢᵢᵢ] has an amino acid sequence of CXXDXXXGCISYC (SEQ ID NO: 35), wherein each “X” is an independently selected amino acid.

Embodiment 37. The oligomeric compound of any of embodiments 1-36, wherein the bicycle ligand comprises at least one, at least two, or at least three non-natural amino acids.

Embodiment 38. The oligomeric compound of embodiment 37, wherein at least one non-natural amino acid is selected from a _{D}-amino acid, allo-isoleucine, 2-amino-3-ethyl-pentanoic acid, aminoisobutyric acid, aminobutyric acid, azetidine, 7-azatryptophan, 6-azidolysine, β-cyclobutylalanine, β-methyl isoleucine, 4, 4-biphenylalanine, cis-hydroxyproline, cyclobutyl glycine, cyclohexyl glycine, cyclopentyl alanine, cyclopentyl glycine, 2,6-dimethyl tyrosine, 3,3-diphenyl alanine, 4-trans-hydroxy-L-proline, 1-napthaylalanine, 2-napthylalanine, N-methyl alanine, 1-methyl histidine, 3-methyl histidine, N-methyl-tryptophan, pipecolic acid,4-pyridylalanine, sarcosine, t-butyl alanine, or 3-t-butyl tyrosine.

Embodiment 39. The oligomeric compound of embodiment 38, wherein at least one non-natural amino acid is selected from 4-trans-hydroxy-L-proline, 6-azidolysine, and t-butyl glycine.

Embodiment 40. The oligomeric compound of any of embodiments 1-39, wherein the molecular scaffold is 1,1′,1″-(1,3,5-triazinane-1,3,5-triyl)triprop-2-en-1-one (TATA).

Embodiment 41. The oligomeric compound of any of embodiments 1-39, wherein the molecular scaffold is 1,1′,1″-(1,3,5-triazinane-1,3,5-triyl)tris(2-bromoethanone) (TATB).

Embodiment 42. The oligomeric compound of any of embodiments 1-41, wherein the bicycle ligand does not inhibit the binding of transferrin to the transferrin receptor.

Embodiment 43. The oligomeric compound of any of embodiments 1-42, wherein at least one nucleoside of the modified oligonucleotide comprises a modified sugar moiety.

Embodiment 44. The oligomeric compound of embodiment 43, wherein at least one modified sugar moiety comprises a bicyclic sugar moiety.

Embodiment 45. The oligomeric compound of embodiment 44, wherein the bicyclic sugar moiety comprises a 2′-4′ bridge selected from —O-CH2-; and —O—CH(CH3)-.

Embodiment 46. The oligomeric compound of embodiment 43-45, wherein at least one modified sugar moiety comprises a non-bicyclic modified sugar moiety.

Embodiment 47. The oligomeric compound of embodiment 46, wherein the non-bicyclic modified sugar moiety is a 2′-MOE sugar moiety or 2′-OMe sugar moiety.

Embodiment 48. The oligomeric compound of any of embodiments 43-47, wherein at least one nucleoside of the modified oligonucleotide compound comprises a sugar surrogate.

Embodiment 49. The oligomeric compound of any of embodiments 1-48, wherein the modified oligonucleotide comprises at least one modified internucleoside linkage.

Embodiment 50. The oligomeric compound of embodiment 49, wherein at least one modified internucleoside linkage is a phosphorothioate internucleoside linkage.

Embodiment 51. The oligomeric compound of any of embodiments 49-50, wherein each internucleoside linkage is a modified internucleoside linkage.

Embodiment 52. The oligomeric compound of embodiment 51, wherein each internucleoside linkage is a phosphorothioate internucleoside linkage.

Embodiment 53. The oligomeric compound of any of embodiments 1-50, wherein the modified oligonucleotide comprises at least one phosphodiester internucleoside linkage.

Embodiment 54. The oligomeric compound of any of embodiments 1-50 or 53, wherein each internucleoside linkage of the modified oligonucleotide is independently selected from a phosphodiester or a phosphorothioate internucleoside linkage.

Embodiment 55. The oligomeric compound of embodiment 49, wherein at least one modified internucleoside linkage is a mesyl phosphoramidate internucleoside linkage.

Embodiment 56. The oligomeric compound of any of embodiments 49-55, wherein each internucleoside linkage is independently selected from a phosphodiester, a phosphorothioate internucleoside, or a mesyl phosphoramidate internucleoside linkage.

Embodiment 57. The oligomeric compound of any of embodiments 49-55, wherein each internucleoside linkage is independently selected from a phosphorothioate internucleoside or a mesyl phosphoramidate internucleoside linkage.

Embodiment 58. The oligomeric compound of any of embodiments 1-57, wherein the modified oligonucleotide comprises at least one modified nucleobase.

Embodiment 59. The oligomeric compound of embodiment 58, wherein the modified nucleobase is 5-methylcytosine.

Embodiment 60. The oligomeric compound of any of embodiments 1-59, wherein the modified oligonucleotide comprises a deoxy region consisting of 5-12 contiguous 2′-deoxynucleosides.

Embodiment 61. The oligomeric compound of embodiment 60, wherein each nucleoside of the deoxy region is a 2′-β-D-deoxynucleoside.

Embodiment 62. The oligomeric compound of embodiment 60 or 61, wherein the deoxy region consists of 7, 8, 9, 10, or 7-10 linked nucleosides.

Embodiment 63. The oligomeric compound of any of embodiments 60-62, wherein each nucleoside immediately adjacent to the deoxy region comprises a modified sugar moiety.

Embodiment 64. The oligomeric compound of any of embodiments 60-63, wherein the deoxy region is flanked on the 5′-side by a 5′-region consisting of 1-6 linked 5′-region nucleosides and on the 3′-side by a 3′-region consisting of 1-6 linked 3′-region nucleosides; wherein the 3′-most nucleoside of the 5′-region comprises a modified sugar moiety; and the 5′-most nucleoside of the 3′-region comprises a modified sugar moiety.

Embodiment 65. The oligomeric compound of embodiment 64, wherein each nucleoside of the 3′-region comprises a modified sugar moiety.

Embodiment 66. The oligomeric compound of embodiment 64 or 65, wherein each nucleoside of the 5′-region comprises a modified sugar moiety.

Embodiment 67. The oligomeric compound of any of embodiments 1-66, wherein the modified oligonucleotide has a sugar motif comprising:
a 5′-region consisting of 1-6 linked 5′-region nucleosides;
an internal region consisting of 6-10 linked internal region nucleosides; and a 3′-region consisting of 1-6 linked 3′-region nucleosides;
wherein each of the 5′-region nucleosides and each of the 3′-region nucleosides comprises a modified sugar moiety; and each of the internal region nucleosides is selected from a 2′-deoxynucleoside and a 2′-substituted nucleoside.

Embodiment 68. The oligomeric compound of embodiment 67, wherein the modified oligonucleotide has a sugar motif comprising:
a 5′-region consisting of 1-6 linked 5′-region nucleosides;
an internal region consisting of 6-10 linked internal region nucleosides; and a 3′-region consisting of 1-6 linked 3′-region nucleosides;
wherein each of the 5′-region nucleosides and each of the 3′-region nucleosides is a cEt nucleoside or a 2′-MOE nucleoside; and each of the internal region nucleosides is a 2′-β-D-deoxynucleoside.

Embodiment 69. The oligomeric compound of any of embodiments 1-59, wherein each nucleoside of the modified oligonucleotide comprises a 2′-sugar moiety.

Embodiment 70. The oligomeric compound of embodiment 69, wherein each 2′-sugar moiety is selected from 2′-OMe, 2′-MOE, or 2′-NMA.

Embodiment 71. The oligomeric compound of embodiment 69 or 70, wherein each nucleoside of the modified oligonucleotide comprises the same 2′-sugar moiety.

Embodiment 72. The oligomeric compound of any of embodiments 1-71, wherein the conjugate linker is cleavable.

Embodiment 73. The oligomeric compound of any of embodiments 1-72, wherein the conjugate linker comprises 1-3 linker nucleosides.

Embodiment 74. The oligomeric compound of any of embodiments 1-72, wherein the conjugate linker does not comprise any linker nucleosides.

Embodiment 75. The oligomeric compound of any of embodiments 1-74, wherein the conjugate group comprises:

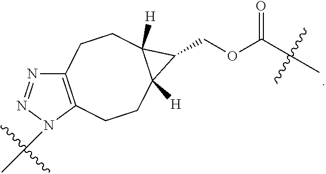 [SMILES: *C(=O)OC[C@@H]1[C@@H]2CCc3c(nnn3*)CC[C@@H]21]

Embodiment 76. The oligomeric compound of any of embodiments 1-74, wherein the conjugate group comprises:

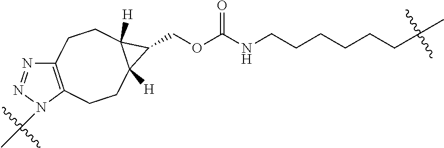 [SMILES: *CCCCCCNC(=O)OC[C@@H]1[C@@H]2CCc3c(nnn3*)CC[C@@H]21]

Embodiment 77. The oligomeric compound of any of embodiments 1-74, wherein the conjugate group comprises:

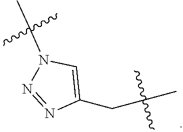 [SMILES: *Cc1cn(*)nn1]

Embodiment 78. The oligomeric compound of any of embodiments 1-74, wherein the conjugate group comprises:

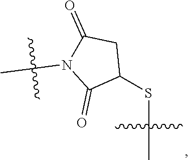 [SMILES: *SC1CC(=O)N(*)C1=O]

optionally

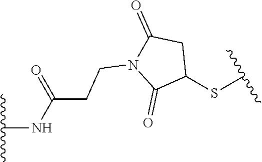 [SMILES: *NC(=O)CCN1C(=O)CC(S*)C1=O]

Embodiment 79. The oligomeric compound of any of embodiments 1-74, wherein the conjugate group comprises:

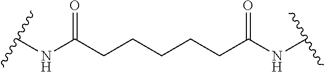 [SMILES: *NC(=O)CCCCCC(=O)N*]

Embodiment 80. The oligomeric compound of any of embodiments 1-74, wherein the conjugate group comprises:

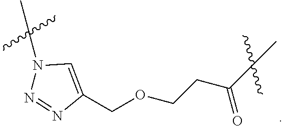 [SMILES: *C(=O)CCOCc1cn(*)nn1]

Embodiment 81. The oligomeric compound of any of embodiments 1-74, wherein the conjugate group comprises:

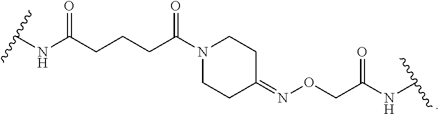 [SMILES: *NC(=O)CCCC(=O)N1CCC(=NOCC(=O)N*)CC1]

Embodiment 82. The oligomeric compound of any of embodiments 1-74, wherein the conjugate group comprises:

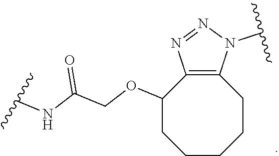 [SMILES: *NC(=O)COC1CCCCCc2c1nnn2*]

Embodiment 83. The oligomeric compound of any of embodiments 1-82, wherein the modified oligonucleotide is complementary to a target nucleic acid expressed in muscle.

Embodiment 84. The oligomeric compound of any of embodiments 1-83, wherein the modified oligonucleotide is capable of reducing the amount of a target nucleic acid through the activation of Rnase H.

Embodiment 85. The oligomeric compound of any of embodiments 1-83, wherein the modified oligonucleotide is capable of reducing the amount of a target nucleic acid through the activation of RISC/Ago2.

Embodiment 86. The oligomeric compound of any of embodiments 1-83, wherein the modified oligonucleotide is capable of modulating the splicing of a target nucleic acid.

Embodiment 87. The oligomeric compound of any of embodiments 1-83, wherein the modified oligonucleotide is a guide RNA, a tracrRNA, or a scout RNA.

Embodiment 88. The oligomeric compound of any of embodiments 1-87, wherein the modified oligonucleotide is complementary to the complement of a target nucleic acid expressed in muscle.

Embodiment 89. The oligomeric compound of any of embodiments 83-88, wherein the target nucleic acid is associated with a muscle disease.

Embodiment 90. The oligomeric compound of any of embodiments 83-89, wherein the target nucleic acid is selected from CaMK2d, NLRP3, PLN, DMD, DMPK, DNM2, DUX4, or HPRT.

Embodiment 91. The oligomeric compound of any of embodiments 83-90, wherein the muscle target nucleic acid has a sequence selected from any of SEQ ID NO: 1-15.

Embodiment 92. The oligomeric compound of any of embodiments 83-91, wherein the target nucleic acid is expressed in at least one of expressed in at least one of the following tissues: skeletal muscle (including but not limited to quadriceps, gastrocnemius, tibialis anterior, triceps, masseter, extensor digitorum longus (EDL), soleus, diaphragm), heart, sciatic nerve, aorta, or liver.

Embodiment 93. The oligomeric compound of any of embodiments 1-92, wherein the nucleobase sequence of the modified oligonucleotide comprises at least 12, at least 13, at least 14, at least 15, or at least 16 contiguous nucleobases of any of the nucleobase sequences of any of SEQ ID NO: 167-191.

Embodiment 94. The oligomeric compound of any of embodiments 1-93, wherein the modified oligonucleotide consists of 10 to 25, 10 to 30, 12 to 20, 12 to 25, 12 to 30, 13 to 20, 13 to 25, 13 to 30, 14 to 20, 14 to 25, 14 to 30, 15 to 20, 15 to 25, 15 to 30, 16 to 18, 16 to 20, 16 to 25, 16 to 30, 17 to 20, 17 to 25, 17 to 30, 18 to 20, 18 to 25, 18 to 30, 19 to 20, 19 to 25, 19 to 30, 20 to 25, 20 to 30, 21 to 25, 21 to 30, 21 to 50, 22 to 25, 22 to 30, 23 to 25, 23 to 30, 20 to 100, 40 to 100, 50 to 100, 50 to 200, 100 to 300, 150-300, or 200-300 linked nucleosides.

Embodiment 95. An oligomeric duplex, comprising a first oligomeric compound comprising a first modified oligonucleotide and a second compound oligomeric comprising a second modified oligonucleotide consisting of 16 to 30 linked nucleosides, wherein the nucleobase sequence of the second modified oligonucleotide comprises a complementary region of at least 12 nucleobases that is at least 90% complementary to an equal length portion of the first modified oligonucleotide, and wherein the second oligomeric compounds is an oligomeric compound of any of embodiments 1-86 or 89-94.

Embodiment 96. The oligomeric duplex of embodiment 95, wherein the first modified oligonucleotide is complementary to a muscle target nucleic acid.

Embodiment 97. The oligomeric duplex of embodiment 96, wherein the duplex is capable of reducing the amount of a target nucleic acid through the activation of RISC/Ago2.

Embodiment 98. The oligomeric duplex of any of embodiments 95-97, wherein at least one nucleoside of the second modified oligonucleotide comprises a modified sugar moiety.

Embodiment 99. The oligomeric duplex of embodiment 98, wherein the modified sugar moiety of the second modified oligonucleotide comprises a bicyclic sugar moiety.

Embodiment 100. The oligomeric duplex of embodiment 99, wherein the bicyclic sugar moiety of the second modified oligonucleotide comprises a 2′-4′ bridge selected from —O—CH₂—; and —O—CH(CH₃)—.

Embodiment 101. The oligomeric duplex of embodiment 100, wherein the modified sugar moiety of the second modified oligonucleotide comprises a non-bicyclic modified sugar moiety.

Embodiment 102. The oligomeric duplex of embodiment 101, wherein the non-bicyclic modified sugar moiety of the second modified oligonucleotide is a 2′-MOE sugar moiety, a 2′-F sugar moiety, or 2′-OMe sugar moiety.

Embodiment 103. The oligomeric duplex of any of embodiments 95-102, wherein at least one nucleoside of the second modified oligonucleotide comprises a sugar surrogate.

Embodiment 104. The oligomeric duplex of any of embodiments 95-102, wherein at least one internucleoside linkage of the second modified oligonucleotide is a modified internucleoside linkage.

Embodiment 105. The oligomeric duplex of embodiment 104, wherein at least one modified internucleoside linkage of the second modified oligonucleotide is a phosphorothioate internucleoside linkage.

Embodiment 106. The oligomeric duplex of any of embodiments 95-105, wherein at least one internucleoside linkage of the second modified oligonucleotide is a phosphodiester internucleoside linkage.

Embodiment 107. The oligomeric duplex of any of embodiments 95-104 or 106, wherein each internucleoside linkage of the second modified oligonucleotide is independently selected from a phosphodiester or a phosphorothioate internucleoside linkage.

Embodiment 108. The oligomeric duplex of any of embodiments 95-107, wherein the second modified oligonucleotide comprises at least one modified nucleobase.

Embodiment 109. The oligomeric duplex of embodiment 108, wherein the modified nucleobase of the second modified oligonucleotide is 5-methylcytosine.

Embodiment 110. The oligomeric duplex of any of embodiments 95-109, wherein the first oligomeric compound comprises a 5′-stabilized phosphate group.

Embodiment 111. The oligomeric duplex of any of embodiments 95-109, wherein the second oligomeric compound comprises a 5′-stabilized phosphate group.

Embodiment 112. The oligomeric duplex of embodiment 110 or 111, wherein the stabilized phosphate group comprises a cyclopropyl phosphonate or a vinyl phosphonate.

Embodiment 113. The oligomeric duplex of any of embodiments 95-112, wherein the first modified oligonucleotide of comprises a glycol nucleic acid (GNA) sugar surrogate.

Embodiment 114. The oligomeric duplex of any of embodiments 95-113, wherein first modified oligonucleotide comprises a 2′-NMA sugar moiety.

Embodiment 115. The oligomeric duplex of any of embodiments 95-114, wherein the second modified oligonucleotide comprises a glycol nucleic acid (GNA) sugar surrogate.

Embodiment 116. The oligomeric duplex of any of embodiments 95-115, wherein the second modified oligonucleotide compound comprises a 2′-NMA sugar moiety.

Embodiment 117. A method of modulating a nucleic acid target in a subject, comprising administering to the subject an oligomeric compound according to any of embodiments 1-94 or an oligomeric duplex of any of embodiments 95-116.

Embodiment 118. The method of embodiment 117, wherein the nucleic acid target is expressed in at least one of skeletal muscle (including but not limited to quadriceps, gastrocnemius, tibialis anterior, triceps, masseter, extensor digitorum longus (EDL), soleus, diaphragm), heart, sciatic nerve, aorta, or liver.

Embodiment 119. The method of embodiment 117 or 118, wherein administration of the oligomeric compound of any of embodiments 1-94 or the oligomeric duplex of any of embodiments 95-116 results in reduction of the nucleic acid target.

Embodiment 120. The method of embodiment 117 or 118, wherein administration of the oligomeric compound of any of embodiments 1-94 or the oligomeric duplex of any of embodiments 95-116 results in a change in the splicing of the nucleic acid target.

Embodiment 121. The method of any of embodiments 117-120, wherein the oligomeric compound or oligomeric duplex is administered via intravenous or subcutaneous dosing.

Embodiment 122. The method of any of embodiments 117-120, wherein the oligomeric compound or oligomeric duplex is administered at a dose of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, or 350 mg.

Embodiment 123. The method of any of embodiments 117-122, comprising administering the oligomeric compound or oligomeric duplex once every 4 weeks, once every 6 weeks, once every 8 weeks, once every 12 weeks, once every 16 weeks, once every 20 weeks, once every 24 weeks, once every 6 months, or once a year.

Embodiment 124. A bicycle ligand specific for transferrin receptor 1 (TfR1) which comprises an amino acid sequence selected from: CP[HyP]DAYLGC[tBuGly]SYCEPWK (SEQ ID NO: 245, herein referred to as BCY21757) and CP[HyP]DAYLGC[tBuGly]SYCEPWC (SEQ ID NO: 246, herein referred to as BCY21758), wherein HyP represents trans-4-hydroxy-L-proline and tBuGly represents t-butyl-glycine.

Embodiment 125. The bicycle ligand of embodiment 124, which comprises an N-terminal acetyl group and a C-terminal CONH₂ group.

Embodiment 126. The bicycle ligand of embodiment 124 or 125, which is a pharmaceutically acceptable salt.

Embodiment 127. The pharmaceutical salt of embodiment 126, wherein the pharmaceutically acceptable salt is selected from the sodium, potassium, calcium or ammonium salt.

Embodiment 128. The bicycle ligand of embodiment 124, wherein the first, second and third cysteine residues within said peptide ligands are covalently bonded to a molecular scaffold such that two polypeptide loops are formed on said molecular scaffold.

Embodiment 129. The bicycle ligand of embodiment 125, wherein the molecular scaffold is 1,1′,1″-(1,3,5-triazinane-1,3,5-triyl)triprop-2-en-1-one (TATA).

### Certain Compounds

In certain embodiments, provided herein are compounds comprising one or more oligonucleotide and one or more conjugate groups. In certain embodiments, an oligonucleotide is a modified oligonucleotide. In certain embodiments, an oligonucleotide is an unmodified oligonucleotide. In certain embodiments, compounds comprise an oligonucleotide, a cell-targeting moiety, and a conjugate linker. In certain embodiments, oligomeric compounds comprise an oligonucleotide, a bicycle ligand, and a conjugate linker. In certain embodiments, oligomeric compounds comprise an oligonucleotide, a polypeptide, a conjugate linker, and optionally N-terminal or C-terminal modifications to the polypeptide. In certain embodiments, oligomeric compounds comprise an oligonucleotide, two or more polypeptides, a branching group, a conjugate linker, and optionally N-terminal or C-terminal modifications to the polypeptides. In certain embodiments, the conjugate linker connects the polypeptide and/or the bicycle ligand to the oligonucleotide.

In certain embodiments, the N-terminus of a bicycle ligand is covalently connected to the conjugate linker, and the conjugate linker is covalently connected to the 3′ end of an oligonucleotide. In certain embodiments, the C-terminus of a bicycle ligand is covalently connected to a conjugate linker, and a conjugate linker is covalently connected to the 3′ end of an oligonucleotide. In certain embodiments, an internal amino acid of a bicycle ligand is covalently connected to a conjugate linker, and a conjugate linker is covalently connected to the 3′ end of an oligonucleotide. In certain embodiments, the N-terminus of a bicycle ligand is covalently connected to a conjugate linker, and the conjugate linker is covalently connected to the 5′ end of an oligonucleotide. In certain embodiments, the C-terminus of a bicycle ligand is covalently connected to an conjugate linker, and the conjugate linker is covalently connected to the 5′ end of an oligonucleotide. In certain embodiments, an internal amino acid of a bicycle ligand is covalently connected to a conjugate linker, and the conjugate linker is covalently connected to the 5′ end of an oligonucleotide. In certain embodiments, the N-terminus of a bicycle ligand is covalently connected to a conjugate linker, and the conjugate linker is covalently connected at an internal position of an oligonucleotide. In certain embodiments, the C-terminus of a bicycle ligand is covalently connected to a conjugate linker, and the conjugate linker is covalently connected at an internal position of an oligonucleotide. In certain embodiments, an internal amino acid of a bicycle ligand is covalently connected to a conjugate linker, and the conjugate linker is covalently connected at an internal position of an oligonucleotide. In certain embodiments, the internal position of an oligonucleotide is the 2′-position of a modified sugar moiety. In certain embodiments, the internal position of an oligonucleotide is a modified internucleoside linkage.

### Certain Conjugate Groups

In certain embodiments, conjugate moieties modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate moieties impart a new property on the attached oligonucleotide.

In certain embodiments, a conjugate group comprises a conjugate moiety and a conjugate linker. In certain embodiments, a conjugate moiety comprises or consists of a cell-targeting moiety. In certain embodiments, a cell-targeting moiety is capable of binding the cell-surface receptor or the cell-surface moiety. In certain embodiments, a compound comprising a cell-targeting moiety is capable of being internalized when it interacts with or binds the cell-surface receptor or the cell-surface moiety. In certain embodiments, a cell-targeting moiety comprises a bicyclic polypeptide or a bicycle ligand. In certain embodiments, a cell-targeting moiety consists of a bicyclic polypeptide or a bicycle ligand.

In certain embodiments, a bicycle ligand comprises a polypeptide comprising at least three reactive groups, separated by at least two loop sequences, and a molecular scaffold which forms covalent bonds with the reactive groups of the polypeptide such that at least two polypeptide loops are formed on the molecular scaffold. In certain embodiments, the molecular scaffold is 1,1′,1″-(1,3,5-triazinane-1,3,5-triyl)triprop-2-en-1-one (TATA). In certain embodiments, the molecular scaffold is 1,1′,1″-(1,3,5-triazinane-1,3,5-triyl)tris(2-bromoethanone) (TATB).

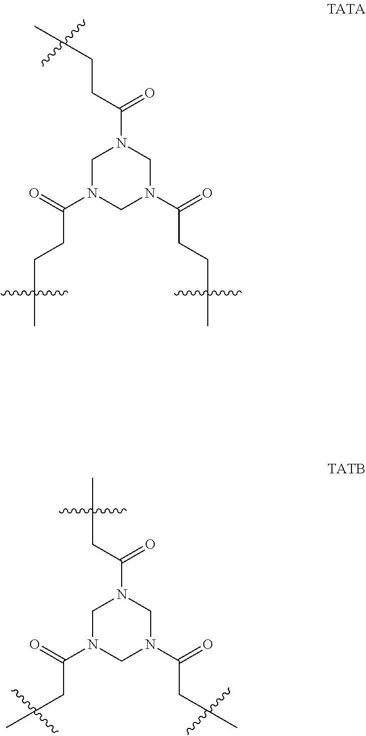 [SMILES: *CCC(=O)N1CN(C(=O)CC*)CN(C(=O)CC*)C1 ; *CC(=O)N1CN(C(=O)C*)CN(C(=O)C*)C1]

In certain embodiments, reactive groups are cysteines. In certain embodiments, loop sequences comprise 2, 3, 4, 5, 6, 7, 8, or 9 amino acids. In certain embodiments, loop sequences comprise three cysteine residues separated by two loop sequences, the first of which consists of 2 amino acids and the second of which consists of 9 amino acids. In certain embodiments, the loop sequences comprise three cysteine residues by two loop sequences both of which consist of 6 amino acids. In certain embodiments, the loop sequences comprise three cysteine residues separated by two loop sequences the first of which consists of 3 amino acids and the second of which consists of 8 amino acids. In certain embodiments, the loop sequences comprise three cysteine residues separated by two loop sequences the first of which consists of 7 amino acids and the second of which consists of 3 amino acids.

In certain embodiments, a bicycle ligand comprises an amino acid sequence selected from:

CᵢXXCᵢᵢXXXXXXXXXCᵢ;
CᵢXXXXXXCᵢᵢXXXXXXCᵢᵢᵢ;
CᵢXXXCᵢᵢXXXXXXXXᵢᵢᵢ;
or
CᵢXXXXXXXCᵢᵢXXXC_{iii;}

wherein Cᵢ, Cᵢᵢ and Cᵢᵢᵢ represent first, second and third cysteine residues, respectively, and each “X” represents an independently selected natural or non-natural amino acid, or a pharmaceutically acceptable salt thereof. In certain embodiments, a bicycle ligand further comprises an N-terminal extension and/or a C-terminal extension.

In certain embodiments, a bicycle ligand comprises an amino acid sequence at least 80%, 85%, 90%, 95%, or 100% identical to any of the following sequences:

(SEQ ID NO: 26)
CᵢSPDAHLGCᵢᵢISYCᵢᵢᵢ;
(SEQ ID NO: 27)
CᵢSPDAYLGCᵢᵢISYCᵢᵢᵢ;
(SEQ ID NO: 93)
CᵢP[HyP]DAYLGCᵢᵢISYCᵢᵢᵢ;
(SEQ ID NO: 95)
CᵢS[HyP]DAHLGCᵢᵢISYCᵢᵢᵢ;
(SEQ ID NO: 128)
CᵢS[Aze]DAHLGCᵢᵢISYCᵢᵢᵢ;
(SEQ ID NO: 86)
CᵢP[HyP]DAYLGCᵢᵢ[tBuGly]SYCᵢᵢᵢ;
(SEQ ID NO: 150)
Cᵢ[K(N₃)]PDAHLGCᵢᵢISYCᵢᵢᵢ;
(SEQ ID NO: 151)
CᵢS[K(N₃)]DAHLGCᵢᵢISYCᵢᵢᵢ;
or
(SEQ ID NO: 152)
CᵢSPD[K(N₃)]HLGCᵢᵢISYCᵢᵢᵢ;

Cᵢ, Cᵢᵢ and Cᵢᵢᵢ represent first, second and third cysteines, respectively; [HyP] represents 4-trans-hydroxy-L-proline; [Aze] represents azetidine; [tBuGly] represents t-butyl glycine; and [K(N₃)] represents 6-azido lysine. In certain embodiments, a bicycle ligand further comprises an N-terminal extension and/or a C-terminal extension.

In certain embodiments, a bicycle ligand comprises an amino acid sequence of: CiXXDXXXGCᵢᵢISYCᵢᵢᵢ (SEQ ID NO: 35); wherein each X is independently selected from natural or non-natural amino acid, or a pharmaceutically acceptable salt thereof. In certain embodiments, a bicycle ligand further comprises an N-terminal extension and/or a C-terminal extension.

In certain embodiments, an oligomeric compound comprises two or more bicyclic ligands attached through a conjugate linker including a bivalent linker including a branching group. In certain embodiments, a bivalent linker comprises one or more PEG repeats. In certain embodiments, the bivalent linker is shown in the structure below:

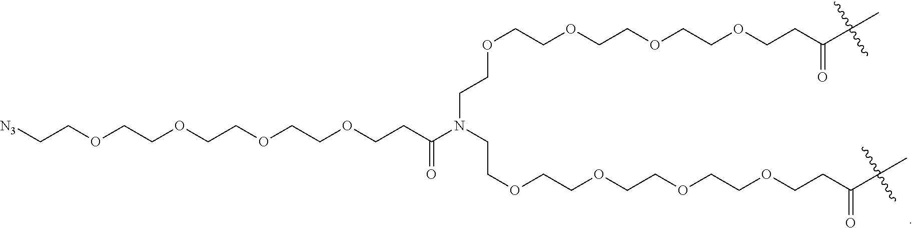 [SMILES: *C(=O)CCOCCOCCOCCOCCN(CCOCCOCCOCCOCCC(*)=O)C(=O)CCOCCOCCOCCOCCN=[N+]=[N-]]

In certain embodiments, aa bicycle ligand is capable of interacting with a cell surface receptor on a cell. In certain embodiments, a bicycle ligand is capable of interacting with a cell surface moiety on a cell. In certain embodiments, a bicycle ligand is capable of binding a cell surface receptor on a cell. In certain embodiments, a bicycle ligand is capable of binding a cell surface moiety on a cell. In certain embodiments, a bicycle ligand is capable of being internalized by the cell when it interacts with or binds a cell surface receptor or cell surface moiety. In certain embodiments, a cell surface receptor is not expressed ubiquitously (e.g., the cell surface receptor is undetectable in at least one tissue of a human subject), and a bicycle ligand selectively delivers an oligonucleotide to a tissue of interest or a cell of interest. By way of non-limiting example, tissue of interest may be any one of brain, spinal cord, retina, heart, kidney, liver, lung, skeletal muscle, cardiac muscle, smooth muscle, adipose, white adipose, brown adipose, spleen, bone, intestine, colon, testes, breast, ovary, placenta, uterus, bladder, pancreas, pituitary, prostate, skin, adrenal gland, and thyroid. By way of non-limiting example, the cell of interest may be any one of a myocyte, adipocyte, hepatocyte, cardiomyocyte, vascular smooth muscle cell, endothelial cell, neuron, blood cell, macrophage, lymphocyte, cancer cell, and immune cell.

In certain embodiments, a bicycle ligand is capable of interacting with or binding a cell surface receptor. In certain embodiments, the cell surface receptor is capable of internalizing the bicycle ligand. In certain embodiments, the cell surface receptor is capable of internalizing an oligonucleotide connected to the bicycle ligand via a conjugate linker. In certain embodiments, the cell surface receptor is human transferrin receptor.

In certain embodiments, a bicycle ligand is represented by the formula [B]n-[Zᵢ]-[J]ₘ—[Zᵢᵢ]—[O]ₒ—[Zᵢᵢᵢ]—[U]ₚ, wherein:
Zᵢ, Zᵢᵢ, and Zᵢᵢᵢ are the first, second, and third amino acids comprising a reactive group; each B, J, O, and U is independently selected amino acids or amino acid mimetics;
n is from 0 to 5;
m is from 3 to 7;
o is from 3 to 7;
p is from 0 to 5;
wherein the sum of m+o is less than 12.

In certain embodiments, a bicycle ligand comprises the following structure,

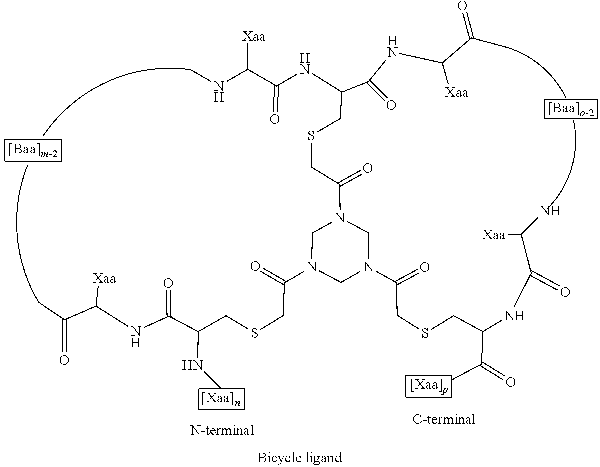

wherein each Xaa is an independently selected amino acid side chain;
each Baa is an independently selected amino acid or amino acid mimetic;
n is from 0 to 5;
m is from 3 to 7;
o is from 3 to 7;
p is from 0 to 5;
wherein the sum of m+o is less than 12.

In certain embodiments, the conjugate linker is attached through the N-terminus, the C-terminus, or through one of the loop amino acids.

In certain embodiments, a bicycle ligand comprises the formula [Zᵢ]-[J]ₘ[Zᵢᵢ]—[O]ₒ—[Zᵢᵢᵢ]. In certain embodiments, a bicycle ligand comprises the following structure:

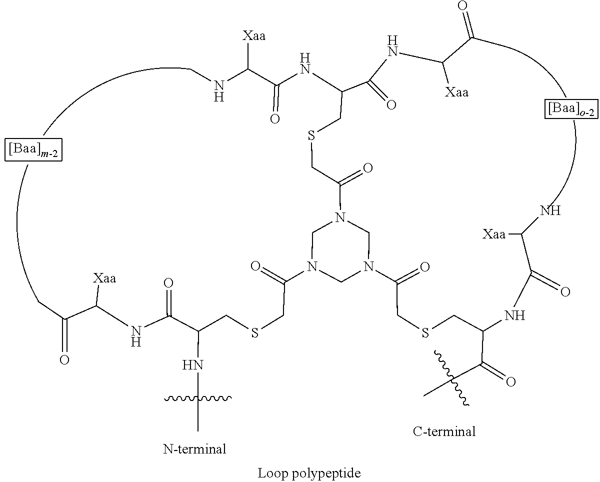

wherein each Xaa is an independently selected amino acid side chain, each Baa is an independently selected amino acid or amino acid mimetic;
m is from 3 to 7 and;
o is from 3 to 7.

In certain embodiments, a bicycle ligand further comprises an N-terminal extension and/or a C-terminal extension.

In certain embodiments, a conjugate linker is attached through the N-terminus, the C-terminus, or through one of the loop amino acids.

In certain embodiments, a bicycle ligand comprises or consists of a sequence selected from the following table:

TABLE 1
Bicycle ligands

| | N- | | |
|---|---|---|---|
| Bicycle | terminal | | SEQ ID |
| Number | Mod. | Sequence | NO: |
| BCY13983 | none | ACSADDWLGCISWCA | 36 |
| BCY14474 | none | ACSADDWLGCISWCA[Sar]₆[K-Fl] | 37 |
| BCY13986 | none | ACSSDAYLGCISWCA | 38 |
| BCY14475 | none | ACSSDAYLGCISWCA[Sar]₆[K-Fl] | 39 |
| BCY15466 | none | ACPPDAHLGCISWCA | 40 |
| BCY15889 | Ac | CPPDAHLGCISWC | 41 |
| BCY15467 | none | ACPQDAYLGCISWCA | 42 |
| BCY15890 | Ac | CPQDAYLGCISWC | 43 |
| BCY13989 | none | ACPPDSWQGCISYCA | 44 |
| BCY14476 | none | ACPPDSWQGCISYCA[Sar]₆[K-Fl] | 45 |
| BCY15468 | none | ACSPDAHLGCISYCA | 46 |
| BCY15768 | none | ACSPDAHLGCISYC[Sar]₆[K-Fl] | 192 |
| BCY15934 | none | CSPDAHLGCISYC[Sar]₆[K-Fl] | 47 |
| BCY15937 | Ac | CSPDAHLGCISYCA[Sar]₆[K-Fl] | 48 |
| BCY15938 | Ac | CSPDAHLGCISYC[Sar]6[K-Fl] | 47 |
| BCY15940 | none | [Fl-G][Sar]₅ACSPDAHLGCISYCA | 49 |
| BCY18030 | none | N[1Nal]NCSPDAHLGCISYC | 50 |
| BCY18039 | Ac | CSPDAHLGCISYCE[Pip]W | 51 |
| BCY17994 | Ac | CSPDAHLGCISYCEPW | 52 |
| BCY18029 | none | NWNCSPDAHLGCISYC | 53 |
| BCY17109 | none | NWNCSPDAHLGCISYCA | 54 |
| BCY18037 | Ac | CSPDAHLGCISYCE[Aze]W | 55 |
| BCY17992 | Ac | NWNCSPDAHLGCISYC | 53 |
| BCY18038 | Ac | CSPDAHLGCISYCE[dP]W | 56 |
| BCY18034 | Ac | N[1Nal]NCSPDAHLGCISYC | 57 |
| BCY18031 | none | N[dW]NCSPDAHLGCISYC | 58 |
| BCY18035 | Ac | N[dW]NCSPDAHLGCISYC | 58 |
| BCY17110 | none | HWMCSPDAHLGCISYCA | 59 |
| BCY17115 | none | ACSPDAHLGCISYCPHP | 60 |
| BCY17114 | none | ACSPDAHLGCISYCEPW | 61 |
| BCY17112 | none | NEVCSPDAHLGCISYCA | 62 |
| BCY17120 | none | ACSPDAHLGCISYCPIVH | 63 |
| BCY15891 | Ac | CSPDAHLGCISYC | 26 |
| BCY17111 | none | HTSCSPDAHLGCISYCA | 64 |
| BCY18036 | Ac | N[NMeTrp]NCSPDAHLGCISYC | 65 |
| BCY18032 | none | N[NMeTrp]NCSPDAHLGCISYC | 65 |
| BCY15939 | Ac | ACSPDAHLGCISYCA | 66 |
| BCY17119 | none | ACSPDAHLGCISYCEHQE | 67 |
| BCY17113 | none | ESFCSPDAHLGCISYCA | 68 |
| BCY17870 | none | NWNCSPDAHLGCISYC[K(N₃)] | 69 |
| BCY17871 | Ac | NWNCSPDAHLGCISYC[K(N₃)] | 69 |
| BCY17872 | AzPro | NWNCSPDAHLGCISYC | 53 |
| BCY17873 | Ac | CSPDAHLGCISYCEPW[K(N₃)] | 70 |
| BCY17874 | AzPro | CSPDAHLGCISYCEPW | 52 |
| BCY17868 | Ac | CSPDAHLGCISYC[K(N₃)] | 71 |
| BCY17869 | AzPro | CSPDAHLGCISYC | 26 |
| BCY17882 | Ac | N[dY]NCSPDAHLGCISYC[K(N₃)] | 72 |
| BCY17890 | Ac | CSPDAHLGCISYCE-[dP]W[K(N₃)] | 73 |
| BCY17892 | Ac | CSPDAHLGCISYCE-[Aze]W[K(N₃)] | 74 |
| BCY17894 | Ac | CSPDAHLGCISYCE-[Pip]W[K(N₃)] | 75 |
| BCY17906 | Ac | CSPDAHLGCISYC[K(N₃)(PYA-maleimide)] | 76 |
| BCY19405 | Ac | CSPDAHLGCISYCEPW[PEG10][K(N₃)] | 77 |
| BCY19406 | Ac | CSPDAHLGCISYCEPW[PEG24][K(N₃)] | 78 |
| BCY19407 | Ac | CSPDAHLGCISYCEPWGGSGGS[K(N₃)] | 79 |
| BCY15469 | none | ACPGDAHLGCISYCA | 80 |
| BCY15892 | Ac | CPGDAHLGCISYC | 81 |
| BCY15470 | none | ACPPDSHLGCISYCA | 82 |
| BCY15893 | Ac | CPPDSHLGCISYC | 83 |
| BCY15471 | none | ACSADDWLGCISYCA | 84 |
| BCY15894 | Ac | CSADDWLGCISYC | 85 |
| BCY17991 | Ac | CP[HyP]DAYLGC[tBuGly]SYC | 86 |
| BCY17995 | Ac | CP[HyP]DAYLGC[tBuGly]SYCEPW | 87 |
| BCY17993 | Ac | NWNCP[HyP]DAYLGC[tBuGly]SYC | 88 |
| BCY16754 | none | ACP[HyP]DAYLGC[tBuGly]SYCA | 89 |
| BCY18033 | none | NWNCP[HyP]DAYLGC[tBuGly]SYC | 88 |
| BCY17896 | Ac | CP[HyP]DAYLGC[tBuGly]SYC[K(N₃)] | 90 |
| BCY17899 | Ac | NWNCP[HyP]DAYLGC[tBuGly]SYC[K(N₃)] | 91 |
| BCY17901 | Ac | CP[HyP]DAYLGC[tBuGly]SYCEPW[K(N₃)] | 92 |
| BCY17990 | Ac | CP[HyP]DAYLGCISYC | 93 |
| BCY17875 | Ac | CP[HyP]DAYLGCISYC[K(N₃)] | 94 |
| BCY17876 | AzPro | CP[HyP]DAYLGCISYC | 93 |
| BCY17989 | Ac | CS[HyP]DAHLGCISYC | 95 |
| BCY16047 | none | ACS[HyP]DAHLGCISYCA | 96 |
| BCY17877 | Ac | CS[HyP]DAHLGCISYC[K(N₃)] | 97 |
| BCY17878 | AzPro | CS[HyP]DAHLGCISYC | 95 |
| BCY16962 | none | ACP[Aib]DAHLGC[tBuGly]SYCA | 98 |
| BCY17117 | none | TYMNCPPDAHLGCISYCA | 99 |
| BCY16048 | none | ACPPDAHLGCISYCA | 100 |
| BCY16963 | none | ACP[Aib]DAYLGC[tBuGly]SYCA | 101 |
| BCY17987 | Ac | CSADAHLGCISYC | 102 |
| BCY16753 | none | ACS[Aib]DAHLGC[tBuGly]SYCA | 103 |
| BCY16046 | none | ACSPDAHLGC[EPA]SYCA | 104 |
| BCY16964 | none | ACPPDAYLGC[tBuGly]SYCA | 105 |
| BCY16965 | none | ACS[Aib]DAYLGC[tBuGly]SYCA | 106 |
| BCY17986 | Ac | CAPDAHLGCISYC | 107 |
| BCY16550 | none | ACP[Aib]DAHLGCISYCA | 108 |
| BCY16966 | none | ACSPDAYLGC[tBuGly]SYCA | 109 |
| BCY16051 | none | ACSPDAHLGC[tBuGly]SYCA | 110 |
| BCY17118 | none | IDSNCPNDAHLGCISYCA | 111 |
| BCY17116 | none | WGKSCPIDAHLGCISYCA | 112 |
| BCY16053 | none | ACSPDAYLGCISYCA | 113 |
| BCY16557 | none | ACPPDAYLGCISYCA | 114 |
| BCY16035 | none | ACS[Aib]DAHLGCISYCA | 115 |
| BCY16043 | none | ACSPDAHLGC[Chg]SYCA | 116 |
| BCY15769 | none | ACAPDAHLGCISYCA[Sar]₆[K-Fl] | 117 |
| BCY15648 | none | ACYLPDW[tBuAla]CGDEYCA | 118 |
| BCY16031 | none | ACSPDAHLGCIS[2Nal]CA | 119 |
| BCY16079 | none | ACSPDAHLGCIS[3tBuTyr]CA | 120 |
| BCY16036 | none | ACSPD[Aib]HLGCISYCA | 121 |
| BCY16029 | none | ACSPDAHLGCIS[1Nal]CA | 122 |
| BCY16089 | none | ACSPDAH[tBuAla]GCISYCA | 123 |
| BCY16088 | none | ACSPDAH[Cba]GCISYCA | 124 |
| BCY16052 | none | ACSPDAHLGCISWCA | 125 |
| BCY16033 | none | ACSPD[Abu]HLGCISYCA | 126 |
| BCY16039 | none | ACS[Aze]DAHLGCISYCA | 127 |
| BCY17988 | Ac | CS[Aze]DAHLGCISYC | 128 |
| BCY17879 | Ac | CS[Aze]DAHLGCISYC[K(N₃)] | 129 |
| BCY17880 | AzPro | CS[Aze]DAHLGCISYC | 128 |
| BCY16038 | Ac | ACSPDDHLGCISYCA | 130 |
| BCY16050 | none | ACSPDSHLGCISYCA | 131 |
| BCY16034 | none | ACSPDAH[Abu]GCISYCA | 132 |
| BCY16032 | none | ACSPDAHLGCIS[4Pal]CA | 133 |
| BCY16049 | none | ACP[dA]DAHLGCISYCA | 134 |
| BCY16558 | none | ACSPDAYLGC[tBuAla]SYCA | 135 |
| BCY16041 | none | ACSPDAHLGC[C5g]SYCA | 136 |
| BCY16042 | none | ACSPDAHLGC[Cbg]SYCA | 137 |
| BCY16045 | none | ACSPDAHL[dA]CISYCA | 138 |
| BCY16037 | none | ACSPDAH[Aib]GCISYCA | 139 |
| BCY16044 | none | ACSPDAHLGC[Cpg]SYCA | 140 |
| BCY16040 | none | ACSPDAHLGC[B-MeIle]SYCA | 141 |
| BCY15771 | none | ACSADAHLGCISYCA[Sar]₆[K-Fl] | 142 |
| BCY15772 | none | ACSPAAHLGCISYCA[Sar]₆[K-Fl] | 143 |
| BCY15773 | none | ACSPDAALGCISYCA[Sar]₆[K-Fl] | 144 |
| BCY15774 | none | ACSPDAHAGCISYCA[Sar]₆[K-Fl] | 145 |
| BCY15775 | none | ACSPDAHLACISYCA[Sar]₆[K-Fl] | 146 |
| BCY15776 | none | ACSPDAHLGCASYCA[Sar]₆[K-Fl] | 147 |
| BCY15777 | none | ACSPDAHLGCIAYCA[Sar]₆[K-Fl] | 148 |
| BCY15770 | none | ACSPDAHLGCISACA[Sar]₆[K-Fl] | 149 |
| BCY17903 | Ac | C[K(N₃)]PDAHLGCISYC | 150 |
| BCY17904 | Ac | CS[K(N₃)]DAHLGCISYC | 151 |
| BCY17905 | Ac | CSPD[K(N₃)]HLGCISYC | 152 |
| BCY23180 | none | C[HyP][HyP]DAYLGC[tBuGly]SYCEPW | 195 |
| BCY21757 | Ac | CP[HyP]DAYLGC[tBuGly]SYCEPWK | 245 |
| BCY21758 | Ac | CP[HyP]DAYLGC[tBuGly]SYCEPWC | 246 |
| BCY23181 | none | C[Oxa][HyP]DAYLGC[tBuGly]SYCEPW | 196 |
| BCY23182 | none | C[Cis-HyP][HyP]DAYLGC[tBuGly]SYCEPW | 197 |
| BCY23183 | none | CP[Oxa]DAYLGC[tBuGly]SYCEPW | 198 |
| BCY23184 | none | CP[Cis-HyP]DAYLGC[tBuGly]SYCEPW | 199 |
| BCY23185 | none | CP[HyP]DA[DOPA]LGC[tBuGly]SYCEPW | 200 |
| BCY23186 | none | CP[HyP]DA[pCaPhe]LGC[tBuGly]SYCEPW | 201 |
| BCY23187 | none | CP[HyP]DA[pCoPhe]LGC[tBuGly]SYCEPW | 202 |
| BCY23188 | none | CP[HyP]DA[hTyr]LGC[tBuGly]SYCEPW | 203 |
| BCY23189 | none | CP[HyP]DAYL[dS]C[tBuGly]SYCEPW | 204 |
| BCY23190 | none | CP[HyP]DAYL[dT]C[tBuGly]SYCEPW | 205 |
| BCY23191 | none | CP[HyP]DAYL[dD]C[tBuGly]SYCEPW | 206 |
| BCY23192 | none | CP[HyP]DAYL[dE]C[tBuGly]SYCEPW | 207 |
| BCY23193 | none | CP[HyP]DAYL[dN]C[tBuGly]SYCEPW | 208 |
| BCY23194 | none | CP[HyP]DAYL[dQ]C[tBuGly]SYCEPW | 209 |
| BCY23195 | none | CP[HyP]DAYL[dY]C[tBuGly]SYCEPW | 210 |
| BCY23196 | none | CP[HyP]DAYLSC[tBuGly]SYCEPW | 211 |
| BCY23197 | none | CP[HyP]DAYLDC[tBuGly]SYCEPW | 212 |
| BCY23198 | none | CP[HyP]DAYLYC[tBuGly]SYCEPW | 213 |
| BCY23199 | none | CP[HyP]DAYLNC[tBuGly]SYCEPW | 214 |
| BCY23200 | none | CP[HyP]DAYLGC[tBuGly]S[DOPA]CEPW | 215 |
| BCY23201 | none | CP[HyP]DAYLGC[tBuGly]S[pCaPhe]CEPW | 216 |
| BCY23202 | none | CP[HyP]DAYLGC[tBuGly]S[pCoPhe]CEPW | 217 |
| BCY23203 | none | CP[HyP]DAYLGC[tBuGly]S[hTyr]CEPW | 218 |
| BCY23204 | none | CP[HyP]DAYLGC[tBuGly]SYCE[HyP]W | 219 |
| BCY23205 | none | CP[HyP]DAYLGC[tBuGly]SYCE[Oxa]W | 220 |
| BCY23206 | none | CP[HyP]DAYLGC[tBuGly]SYCE[Cis-HyP]W | 221 |
| BCY23207 | none | CP[HyP]DAYLGC[tBuGly]SYCEPY | 222 |
| BCY23208 | none | CP[HyP]DAYLGC[tBuGly]SYCEP[DOPA] | 223 |
| BCY23209 | none | CP[HyP]DAYLGC[tBuGly]SYCEP[pCaPhe] | 224 |
| BCY23210 | none | CP[HyP]DAYLGC[tBuGly]SYCEP[pCoPhe] | 225 |
| BCY23211 | none | CP[HyP]DAYLGC[tBuGly]SYCEP[hTyr] | 226 |
| BCY23216 | none | CP[HyP]EAYLGC[tBuGly]SYCEPW | 227 |
| BCY23217 | none | CP[HyP][Gla]AYLGC[tBuGly]SYCEPW | 228 |
| BCY23218 | none | CP[HyP]DAYSGC[tBuGly]SYCEPW | 229 |
| BCY23219 | none | CP[HyP]DAYTGC[tBuGly]SYCEPW | 230 |
| BCY23220 | none | CP[HyP]DAYDGC[tBuGly]SYCEPW | 231 |
| BCY23221 | none | CP[HyP]DAYEGC[tBuGly]SYCEPW | 232 |
| BCY23222 | none | CP[HyP]DAYNGC[tBuGly]SYCEPW | 233 |
| BCY23223 | none | CP[HyP]DAYQGC[tBuGly]SYCEPW | 234 |
| BCY23224 | none | CP[HyP]DAYLGC[tBuGly][HSer]YCEPW | 235 |
| BCY23225 | none | CP[HyP]DAYLGC[tBuGly]TYCEPW | 236 |
| BCY23226 | none | CP[HyP]DAYLGC[tBuGly]DYCEPW | 237 |
| BCY23227 | none | CP[HyP]DAYLGC[tBuGly]EYCEPW | 238 |
| BCY23228 | none | CP[HyP]DAYLGC[tBuGly]NYCEPW | 239 |
| BCY23229 | none | CP[HyP]DAYLGC[tBuGly]QYCEPW | 240 |
| BCY23230 | none | CP[HyP]DAYLGC[tBuGly]SYCDPW | 241 |
| BCY23231 | none | CP[HyP]DAYLGC[tBuGly]SYC[Gla]PW | 242 |
| BCY23514 | none | CP[HyP]DAYLGCYSYCEPW | 243 |
| BCY23515 | none | CP[HyP]DAYLGC[3HyV]SYCEPW | 244 |

wherein Ac represents acetyl, AzPro represents azidopropyl, Abu represents aminobutyric acid, Aib represents aminoisobutyric acid, Aze represents azetidine, B-MeIle represents beta-methyl isoleucine, C5g represents cyclopentyl glycine, Cba represents β-cyclobutylalanine, Cbg represents cyclobutyl glycine, Chg represents cyclohexyl glycine, Cpg represents cyclopropyl glycine, EPA represents 2-amino-3-ethyl-pentanoic acid, HyP represents trans-4-hydroxy-L-proline, K(N₃) represents 6-azido lysine, 1Nal represents 1-naphthylalanine, 2Nal represents 2-naphthylalanine, 4Pal represents 4-pyridylalanine, Pip represents pipecolic acid: tBuAla represents t-butyl-alanine, tBuGly represents t-butyl-glycine, 3tBuTyr represents 3-t-Butyl-Tyrosine, Sar represents sarcosine, K-Fl represents fluorescein attached at the 6-position of a lysine, Fl-G represents fluorescein attached at the N-terminus of a glycine, NMeTrp represents N-methyl tryptophan, dP represents _{D}-proline, dA represents _{D}-alanine, dW represents _{D}-tryptophan, dS represents _{D}-serine, dT represents _{D}-threonine, dD represents _{D}-aspartic acid, dN represents _{D}-asparagine, dQ represents _{D}-glutamine, Cis-HyP represents cis-L-4-hydroxyproline, DOPA represents 3,4-dihydroxy-phenylalanine, Gla represents L-γ-carboxyglutamic acid, HSer represents homoserine, hTyr represents homo-tyrosine, 3HyV represents 3-hydroxy-L-valine, Oxa represents oxazolidine-4-carboxylic acid, pCaPhe represents L-4-carbamoylphenylalanine, pCoPhe represents 4-carboxy-L-phenylalanine, and [K(N₃)(PYA-Maleimide)] represents a modified lysine having the following structure:

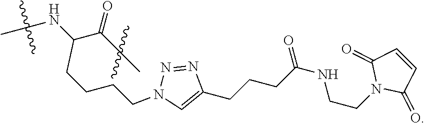 [SMILES: *NC(CCCCn1cc(CCCC(=O)NCCN2C(=O)C=CC2=O)nn1)C(*)=O]

In certain embodiments, a bicycle ligand has the structure:

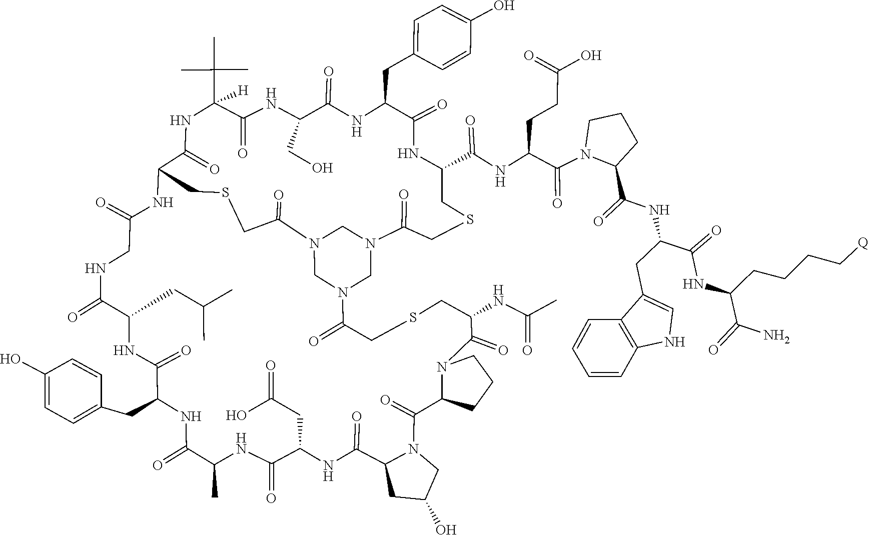

or a salt thereof, wherein Q is N₃ (BCY17901, SEQ ID NO: 92), NH₂ (BCY21758, SEQ ID NO: 245), SH (BCY21758, SEQ ID NO: 246), a conjugate linker, or a conjugate linker covalently connected to an oligonucleotide.

In certain embodiments, a bicycle ligand comprises an amino acid sequence which is selected from CP[HyP]DAYLGC[tBuGly]SYCEPWK (SEQ ID NO: 245, herein referred to as BCY21757) and CP[HyP]DAYLGC[tBuGly]SYCEPWC (SEQ ID NO: 246, herein referred to as BCY21758), wherein HyP represents trans-4-hydroxy-L-proline and tBuGly represents t-butyl-glycine. In certain embodiments, the bicycle ligand comprises an N-terminal acetyl group and a C-terminal CONH₂ group. In certain embodiments, the first, second, and third cysteine residues within the bicycle ligand are covalently bonded to a molecular scaffold such that two polypeptide loops are formed on the molecular scaffold. In certain embodiments, the molecular scaffold is 1,1′,1″-(1,3,5-triazinane-1,3,5-triyl)triprop-2-en-1-one (TATA).

### Transferrin Receptor

In certain embodiments, a bicycle ligand is capable of interacting with the type 1 transferrin receptor. In certain embodiments, a bicycle ligand is capable of binding the type 1 transferrin receptor. In certain embodiments, a bicycle ligand is capable of binding the type 1 transferrin receptor while not interfering with the binding of the natural ligand transferrin. In certain embodiments, a bicycle ligand inhibits the binding of the natural ligand transferrin.

### Certain Conjugate Linkers

In certain embodiments, oligomeric compounds comprise an oligonucleotide and a conjugate group, wherein the conjugate group comprises a conjugate moiety and a conjugate linker. In certain embodiments, the conjugate linker links the conjugate moiety to the oligonucleotide. In certain embodiments, the conjugate linker is a single chemical bond (i.e., the conjugate moiety is attached directly to an oligonucleotide through a single bond). In certain embodiments, the conjugate linker comprises one or more atoms. In certain embodiments, the conjugate linker comprises a chemical group. In certain embodiments, the conjugate linker comprises a chain structure, such as a hydrocarbyl chain, or an oligomer of repeating units such as ethylene glycol, nucleosides, or amino acid units. In certain embodiments, the oligonucleotide is a modified oligonucleotide. In certain embodiments, the conjugate moiety is a bicycle ligand. In certain embodiments, the conjugate moiety comprises two polypeptide loops attached to a molecular scaffold.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate moieties to parent compounds, such as the oligonucleotides provided herein. In general, a bifunctional linking moiety comprises at least two functional groups. One of the functional groups is selected to react with a particular site on a parent compound and the other is selected to react with a peptide extender. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

In certain embodiments, conjugate linkers comprise chemical groups that are formed upon a reaction between a first functional group and a second functional group. In certain embodiments, a modified oligonucleotide is attached to the first functional group during synthesis, and a conjugate moiety is attached to a second functional group during synthesis. Then, the two compounds are mixed under specific conditions to yield the final oligomeric compound. In certain embodiments, the conjugate moiety is a bicycle ligand. In certain embodiments, the conjugate moiety comprises two polypeptide loops attached to a molecular scaffold. Such reactions that are compatible with both oligonucleotide and peptide chemistry have been previously described and are often called “bioconjugation” reactions. These reactions include strain promoted azido-alkyne cycloaddition (SPAAC), copper-catalyzed click reaction (CuAAC), active ester conjugation to an amino modified oligonucleotide, maleimide-thiol Michael addition, ketol/hydroxylamine ligation, the Staudinger ligation, reductive amination, thio ether formation, disulfide formation, reductive alkylation, catalyst-free N-arylation, sulfur fluoride exchange click reaction (SuFEx), and inverse demand Diels Alder reaction. Certain such reactions are described in, e.g., Jbara, et al., “Oligonucleotide Bioconjugation with Bifunctional Palladium Reagents”, Angew. Chem. Int. Ed. 2021, 60(21)12109-12115; Dong, et al., “Sulfur(VI) Fluoride Exchange (SuFEx): Another Good Reaction for Click Chemistry,” Angew. Chem. Int. Ed. 2014, 53(36):9430-9448.4; Zhang, et al., “Arylation Chemistry for Bioconjugation,” Angew. Chem. Int. Ed. Engl. 2019; 58(15): 4810-4839; Walsh, et al., “Site-selective modification strategies in antibody-drug conjugates” Chem. Soc. Rev., 2021, 50: 1305-1353; Tiefenbrunn, et al., “Chemoselective ligation techniques: modern applications of time-honored chemistry”, Biopolymers, 2010, 94(1):95-106; Drake, et al., Bioconjug. Chem. 2014, 25(7):1331-1341; Bode, Acc. Chem. Res., 2017, 50, 9, 2104-2115; J. Magano, B. Bock, et al, Org. Proc. Res. Dev. 2014, 18:142-151; Craig S. McKay and M. G. Finn, “Click Chemistry in Complex Mixtures: Bioorthogonal Bioconjugation”, Chemistry & Biology 2014; Mitchell P. Christy et al., Org. Lett. 2020, 22: 2365; Ren et al., Angew. Chem. Int. Ed. Engl. 2009, 48, 9658-9662; Rohrbacher, F. et al., Helv. Chim. Acta. 2018, 101; Baalmaan, et al, “A Bioorthogonal Click Chemistry Toolbox for Targeted Synthesis of Branched and Well-Defined Protein-Protein Conjugates”, Angew. Chem. Int. Ed. 2020 (59): 12885-12893; Lang, et al, “Biorthogonal Reactions for Labeling Proteins”, J. Am. Chem. Soc, 2014, 9(1):16-20; Nair, et al., “The Thiol-Michael Addition Click Reaction: A Powerful and Widely Used Tool in Materials Chemistry”, Chem. Mater. 2013 26(1):724-744; Kalia and Raines, “Hydrolytic Stability of Hydrazones and Oximes”, Angew. Chem. Int. Ed., 2008, 47:7523-7526.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, the bicycle ligand comprises an N-terminal or a C-terminal extension azide group, which optionally may be joined with an oligomeric compound by cycloaddition with bicyclo[6.1.0]non-4-yn-9-ylmethyl carbamate-oligo or 2-(cyclooct-2-yn-1-yloxy)acetamide-oligo. In certain embodiments, the bicycle ligand comprises an N-terminal or a C-terminal extension amide group which optionally may be joined with an oligomeric compound by coupling with oligo-7-amido-7-oxoheptanoic acid. In certain embodiments, the bicycle ligand comprises an N-terminal or a C-terminal extension 2-(aminooxy)acetamide group which optionally may be joined with an oligomeric compound by condensation with 5-oxo-5-(4-oxopiperidin-1-yl)pentanamide-oligo. In certain embodiments, the bicycle ligand comprises an N-terminal or a C-terminal extension thiol group which optionally may be joined with an oligomeric compound by addition to 3-(2,5-dioxo-2,5-dihydro-1H-pyrrol-1-yl)propenamide-oligo.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) an oligonucleotide consisting of 8-30 nucleosides and (2) a conjugate linker comprising 1-10 linker-nucleosides that are contiguous with the nucleosides of the oligonucleotide. The total number of contiguous linked nucleosides in such an oligomeric compound is more than 30. Alternatively, an oligomeric compound may comprise an oligonucleotide consisting of 8-30 nucleosides and no conjugate linker. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate moiety to be cleaved from the oligonucleotide. For example, in certain circumstances oligomeric compounds comprising a particular conjugate moiety are better taken up by a particular cell type, but once the oligomeric compound has been taken up, it is desirable that the conjugate moiety be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphodiester linkage between an oligonucleotide and a conjugate moiety.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, oligomeric compounds disclosed herein comprise an oligonucleotide linked to conjugate moiety by a conjugate linker, wherein the oligomeric compound is prepared using Click chemistry known in the art. Compounds have been prepared using Click chemistry wherein alkynyl phosphonate internucleoside linkages on an oligomeric compound attached to a solid support are converted into the 1,2,3-triazolylphosphonate internucleoside linkages and then cleaved from the solid support (Krishna et al., J. Am. Chem. Soc. 2012, 134(28), 11618-11631), which is incorporated by reference herein in its entirety. Additional conjugate linkers suitable for use in several embodiments can be prepared by Click chemistry described in “Click Chemistry for Biotechnology and Materials Science” Ed. Joerg Laham, Wiley 2009, which is incorporated by reference herein in its entirety.

In certain embodiments, a Click reaction can be used to link a conjugate moiety and an oligonucleotide by reacting:

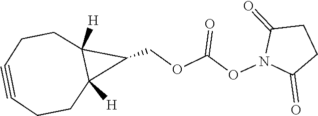 [SMILES: O=C(OC[C@@H]1[C@@H]2CCC#CCC[C@@H]21)ON1C(=O)CCC1=O]

with an oligonucleotide having a terminal amine, including but not limited to the following compound:

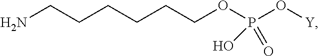

wherein Y is the remainder of the oligonucleotide, to yield:

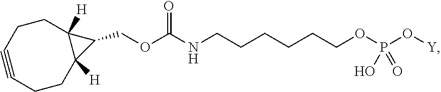

which can be reacted with a conjugate moiety having an azide to yield:

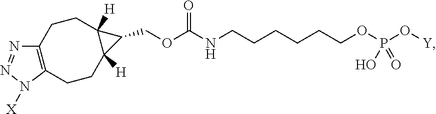

wherein N—N═N is formed from an azido group of the conjugate moiety, and wherein X represents the remainder of the conjugate moiety. In certain embodiments, the conjugate moiety comprises a bicycle ligand.

In certain embodiments, the conjugate moiety comprises a polypeptide. In certain embodiments, the azido group is attached to an amino-acid side chain of the polypeptide. In certain embodiments, the azido group is attached to the N-terminus of the polypeptide. In certain embodiments, the azido group replaces the amino group of a lysine of the polypeptide.

In certain embodiments, an oligomeric compound comprises an oligonucleotide linked to a conjugate moiety by a conjugate linker, wherein the conjugate linker is prepared from the following compound:

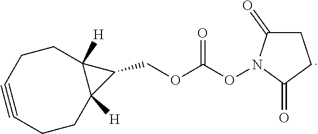 [SMILES: O=C(OC[C@@H]1[C@@H]2CCC#CCC[C@@H]21)ON1C(=O)CCC1=O]

In certain embodiments, an oligomeric compound comprises an oligonucleotide linked to a conjugate moiety by a conjugate linker, wherein the conjugate linker comprises:

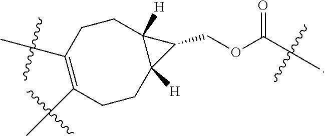 [SMILES: *C(=O)OC[C@@H]1[C@@H]2CC/C(*)=C(/*)CC[C@@H]21]

In certain embodiments, an oligomeric compound comprises an oligonucleotide linked to a conjugate moiety by a conjugate linker, wherein the conjugate linker comprises:

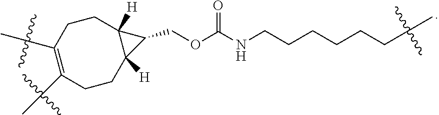 [SMILES: *CCCCCCNC(=O)OC[C@@H]1[C@@H]2CC/C(*)=C(/*)CC[C@@H]21]

In certain embodiments, an oligomeric compound comprises an oligonucleotide linked to a conjugate moiety by a conjugate linker, wherein the compound comprises:

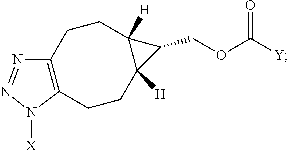

wherein N—N═N is formed from an azido group of the conjugate moiety; X represents the remainder of the conjugate moiety; and Y represents a portion of the oligomeric compound comprising the oligonucleotide. In certain embodiments, the conjugate moiety comprises a bicycle ligand. In certain embodiments, the conjugate moiety comprises a polypeptide. In certain embodiments, the azido group is attached to an amino-acid side chain of the polypeptide. In certain embodiments, the azido group is attached to the N-terminus of the polypeptide. In certain embodiments, the azido group replaces the amino group of a lysine of the polypeptide.

In certain embodiments, an oligomeric compound comprises an oligonucleotide linked to a conjugate moiety by a conjugate linker, wherein the oligomeric compound comprises:

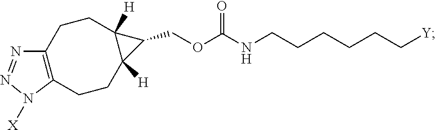

wherein N—N═N is formed from an azido group of the conjugate moiety; X represents the remainder of the conjugate moiety; and Y represents the remainder of the oligonucleotide. In certain embodiments, the conjugate moiety comprises a bicycle ligand. In certain embodiments, the conjugate moiety comprises a polypeptide. In certain embodiments, the azido group is attached to an amino-acid side chain of the polypeptide. In certain embodiments, the azido group is attached to the N-terminus of the polypeptide. In certain embodiments, the azido group replaces the amino group of a lysine of the polypeptide.

In certain embodiments, an oligomeric compound comprises an oligonucleotide linked to a conjugate moiety by a conjugate linker, wherein the oligomeric compound comprises:

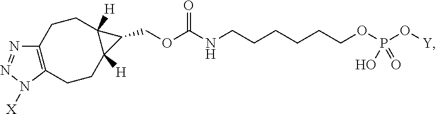

wherein N—N═N is formed from an azido group of the conjugate moiety; X represents the remainder of the conjugate moiety; and Y represents the remainder of the oligonucleotide. In certain embodiments, the conjugate moiety comprises a bicycle ligand. In certain embodiments, the conjugate moiety comprises a polypeptide. In certain embodiments, the azido group is attached to an amino-acid side chain of the polypeptide. In certain embodiments, the azido group is attached to the N-terminus of the polypeptide. In certain embodiments, the azido group replaces the amino group of a lysine of the polypeptide.

In certain embodiments, a Click reaction can be used to link a conjugate moiety and an oligonucleotide by reacting:

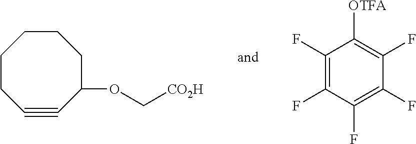

and F in solution together
with an oligonucleotide having a terminal amine, including but not limited to the following compound:

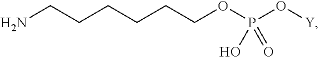

wherein Y is the remainder of the oligonucleotide, to yield:

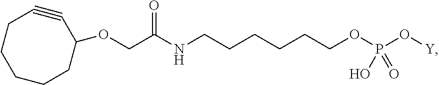

which can be reacted with a conjugate moiety having an azide to yield:

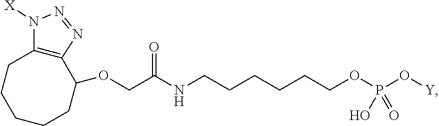

wherein N—N═N is formed from an azido group of the conjugate moiety, and wherein X represents the remainder of the conjugate moiety. In certain embodiments, the conjugate moiety comprises a bicycle ligand. In certain embodiments, the conjugate moiety comprises a polypeptide. In certain embodiments, the azido group is attached to an amino-acid side chain of the polypeptide. In certain embodiments, the azido group is attached to the N-terminus of the polypeptide. In certain embodiments, the azido group replaces the amino group of a lysine of the polypeptide.

In certain embodiments, an oligomeric compound comprises an oligonucleotide linked to a conjugate moiety by a conjugate linker, wherein the conjugate linker is prepared from the following compound:

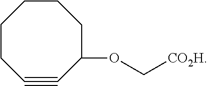 [SMILES: O=C(O)COC1C#CCCCCC1]

In certain embodiments, an oligomeric compound comprises an oligonucleotide linked to a conjugate moiety by a conjugate linker, wherein the conjugate linker comprises:

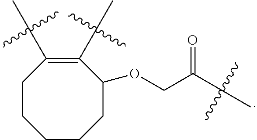 [SMILES: *C(=O)COC1CCCCC/C(*)=C\1*]

In certain embodiments, an oligomeric compound comprises an oligonucleotide linked to a conjugate moiety by a conjugate linker, wherein the conjugate linker comprises:

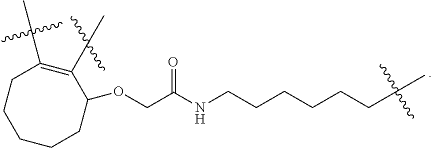 [SMILES: *CCCCCCNC(=O)COC1CCCCC/C(*)=C\1*]

In certain embodiments, an oligomeric compound comprises an oligonucleotide linked to a conjugate moiety by a conjugate linker, wherein the compound comprises:

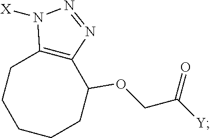

wherein N—N═N is formed from an azido group of the conjugate moiety; X represents the remainder of the conjugate moiety; and Y represents a portion of the oligomeric compound comprising the oligonucleotide. In certain embodiments, the conjugate moiety comprises a bicycle ligand. In certain embodiments, the conjugate moiety comprises a polypeptide. In certain embodiments, the azido group is attached to an amino-acid side chain of the polypeptide. In certain embodiments, the azido group is attached to the N-terminus of the polypeptide. In certain embodiments, the azido group replaces the amino group of a lysine of the polypeptide.

In certain embodiments, an oligomeric compound comprises an oligonucleotide linked to a conjugate moiety by a conjugate linker, wherein the oligomeric compound comprises:

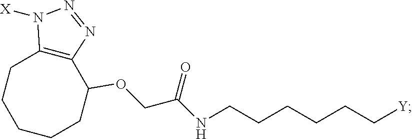

wherein N—N═N is formed from an azido group of the conjugate moiety; X represents the remainder of the conjugate moiety; and Y represents the remainder of the oligonucleotide. In certain embodiments, the conjugate moiety comprises a bicycle ligand. In certain embodiments, the conjugate moiety comprises a polypeptide. In certain embodiments, the azido group is attached to an amino-acid side chain of the polypeptide. In certain embodiments, the azido group is attached to the N-terminus of the polypeptide. In certain embodiments, the azido group replaces the amino group of a lysine of the polypeptide.

In certain embodiments, an oligomeric compound comprises an oligonucleotide linked to a conjugate moiety by a conjugate linker, wherein the oligomeric compound comprises:

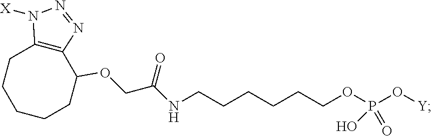

wherein N—N═N is formed from an azido group of the conjugate moiety; X represents the remainder of the conjugate moiety; and Y represents the remainder of the oligonucleotide. In certain embodiments, the conjugate moiety comprises a bicycle ligand. In certain embodiments, the conjugate moiety comprises a polypeptide. In certain embodiments, the azido group is attached to an amino-acid side chain of the polypeptide. In certain embodiments, the azido group is attached to the N-terminus of the polypeptide. In certain embodiments, the azido group replaces the amino group of a lysine of the polypeptide.

In certain embodiments, an oligomeric compound comprises an oligonucleotide linked to a conjugate moiety by a conjugate linker, wherein the oligomeric compound comprises:

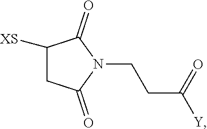

wherein X comprises the conjugate moiety; and Y comprises the oligonucleotide.

In certain embodiments, an oligomeric compound comprises an oligonucleotide linked to a conjugate moiety by a conjugate linker, wherein the oligomeric compound comprises:

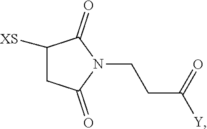

wherein X comprises the oligonucleotide; and Y comprises the conjugate moiety.

In certain embodiments, an oligomeric compound comprises an oligonucleotide linked to a conjugate moiety by a conjugate linker, wherein the conjugate linker comprises:

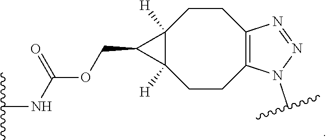 [SMILES: *NC(=O)OC[C@@H]1[C@@H]2CCc3c(nnn3*)CC[C@@H]21]

In certain embodiments, an oligomeric compound comprises an oligonucleotide linked to a conjugate moiety by a conjugate linker, wherein the conjugate linker comprises:

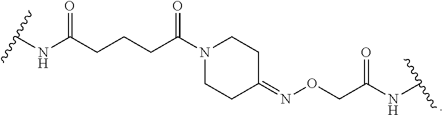 [SMILES: *NC(=O)CCCC(=O)N1CCC(=NOCC(=O)N*)CC1]

In certain embodiments, an oligomeric compound comprises an oligonucleotide linked to a conjugate moiety by a conjugate linker, wherein the conjugate linker comprises:

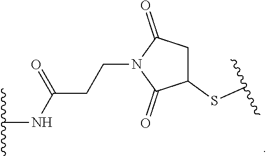 [SMILES: *NC(=O)CCN1C(=O)CC(S*)C1=O]

In certain embodiments, an oligomeric compound comprises an oligonucleotide linked to a conjugate moiety by a conjugate linker, wherein the conjugate linker comprises:

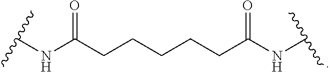 [SMILES: *NC(=O)CCCCCC(=O)N*]

In certain embodiments, an oligomeric compound comprises an oligonucleotide linked to a conjugate moiety by a conjugate linker, wherein the conjugate linker comprises:

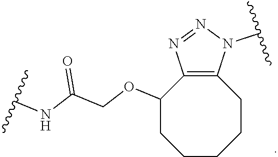 [SMILES: *NC(=O)COC1CCCCCc2c1nnn2*]

Synthetic methods describing preparation of the above starting materials and intermediates can be found in one or more of the following: Agard, et al., “A Strain-Promoted [3+2] Azide-Alkyne Cycloaddition for Covalent Modification of Biomolecules in Living Systems.” J. Am. Chem. Soc. 2004, 126:15046-15047; Lang, et al, “Biorthogonal Reactions for Labeling Proteins”, J. Am. Chem. Soc, 2014, 9(1):16-20; Nair, et al., “The Thiol-Michael Addition Click Reaction: A Powerful and Widely Used Tool in Materials Chemistry”, Chem. Mater. 2013 26(1):724-744; WO2011/136645; Komel and Kool, “Oximes and Hydrazones in Bioconjugation: Mechanism and Catalysis, Chem. Rev., 2017, 117:10358-10376; Wang, et al., “Polyfluorophenyl Ester-Terminated Homobifunctional CrossLinkers for Protein Conjugation”, Synlett, 2017, 28: 1934-1938; Kishimoto, et al, “Site-Specific Chemical Conjugation of Antibodies by Using Affinity Peptide for the Development of Therapeutic Antibody Format”, Bioconj. Chem., 2019, 30:698-702.

### Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### Certain Modified Nucleosides

Modified nucleosides comprise a modified sugar moiety or a modified nucleobase or both a modified sugar moiety and a modified nucleobase.

### Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted non-bicyclic nucleoside modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′—(CH₂)₂-2′, 4′—(CH₂)₃-2′, 4′—CH₂—O-2′ (“LNA”), 4′-CH₂—S-2′, 4′—(CH₂)₂—O-2′ (“ENA”), 4′-CH(CH₃)—O-2′ (referred to as “constrained ethyl” or “cEt”), 4′-CH₂—O—CH₂-2′, 4′—CH₂—N(R)-2′, 4′—CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′C(—RₐR_{b})—N(R)—O-2′, 4′—C(RₐR_{b})—O—N(R)-2′, 4′—CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, C(═NRₐ)—, —C(═O)—, —C(═S)—, —O—, —Si(Rₐ)₂—, —S(═O)ₓ—, and —N(Rₐ)—;
wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

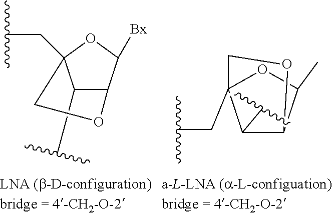

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

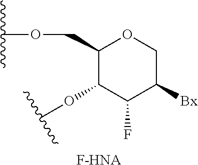

(“F-HNA”, see e.g., Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

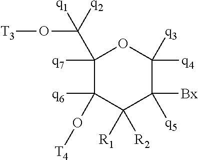

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate moiety, or a 5′ or 3-terminal group;
   q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is 0, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

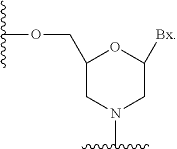

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides (see for example review article: Leumann, Bioorg. Med. Chem., 2002, 10, 841-854).

### Certain Modified Nucleobases

In certain embodiments, modified oligonucleotides comprise one or more nucleosides comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphodiesters (“P═O”) (also referred to as unmodified or naturally occurring linkages or phosphate linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO2021/030778, incorporated by reference herein. In certain embodiments, a modified internucleoside linkage comprises the formula:

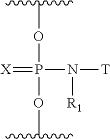

wherein independently for each internucleoside linking group of the modified oligonucleotide:
X is selected from 0 or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

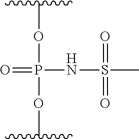 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

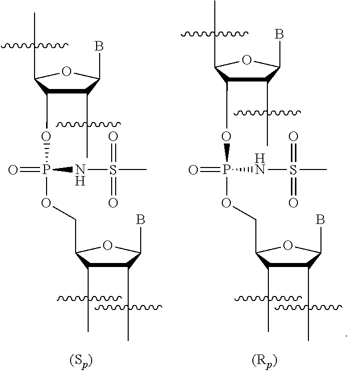

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate or mesyl phosphoramidate linkage. Nonetheless, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

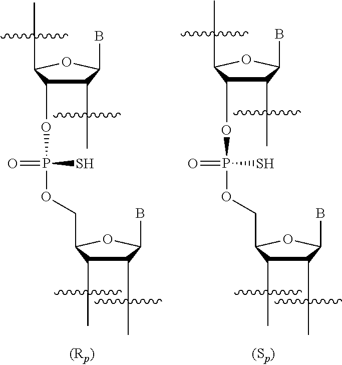

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

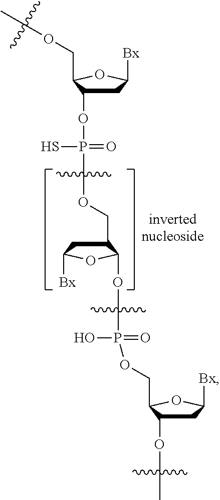

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

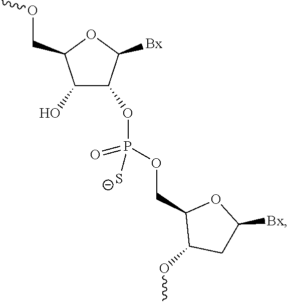

wherein each Bx represents any nucleobase.

### Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

### Gapmers

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric 20 gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap of a gapmer comprises 7-12 nucleosides. In certain embodiments, each nucleoside of the gap of a gapmer is an unmodified 2′-deoxy nucleoside.

In certain embodiments, the gapmer is a deoxy gapmer. In embodiments, the nucleosides on the gap side of each wing/gap junction are unmodified 2′-deoxy nucleosides and the nucleosides on the wing sides of each wing/gap junction are modified nucleosides. In certain embodiments, each nucleoside of the gap is an unmodified 2′-deoxy nucleoside. In certain embodiments, each nucleoside of each wing of a gapmer is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, each nucleoside of the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

Herein, the lengths (number of nucleosides) of the three regions of a gapmer may be provided using the notation [# of nucleosides in the 5′-wing]-[# of nucleosides in the gap]-[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified deoxynucleoside sugars. Thus, a 5-10-5 MOE gapmer consists of 5 linked MOE modified nucleosides in the 5′-wing, 10 linked deoxynucleosides in the gap, and 5 linked MOE nucleosides in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

### Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

### Certain Lengths

It is possible to increase or decrease the length of an oligonucleotide without eliminating activity. For example, in Woolf et al. (Proc. Natl. Acad. Sci. USA 89:7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

### Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for f-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists essentially of a modified or unmodified oligonucleotide, a conjugate linker, and a conjugate moiety. In certain embodiments, the first oligomeric compound of an oligomeric duplex comprises or consists essentially of a modified or unmodified oligonucleotide. In certain embodiments, the second oligomeric compound of an oligomeric duplex comprises or consists essentially of a modified or unmodified oligonucleotide, a conjugate linker and a conjugate moiety. Either or both oligomeric compounds of an oligomeric duplex may comprise a conjugate linker, and a conjugate moiety. In certain embodiments, the oligomeric compound is directly connected to the conjugate linker, the conjugate linker is directly connected to the conjugate moiety. The oligonucleotides of each oligomeric compound of an oligomeric duplex may include non-complementary overhanging nucleosides. In certain embodiments, an overhanging nucleoside may be complementary to the target nucleic acid. In certain embodiments, an overhanging nucleoside is not to a target nucleic acid. In certain embodiments, the two oligonucleotides have at least one mismatch relative to one another. In certain embodiments, the oligomeric duplex is an antisense agent.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide comprises a complementary region of at least 9, at least 10, at least 11, at least 12, at least 13, at least 14, at least 15, at least 16, at least 17, at least 18, at least 19, at least 20, or 21 nucleobases that is at least 90% complementary to the nucleobase sequence of an equal portion of the first modified oligonucleotide. In certain embodiments, the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is at least 95% complementary to the nucleobase sequence of an equal portion of the first modified oligonucleotide. In certain embodiments, the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is 100% complementary to the nucleobase sequence of an equal portion of the first modified oligonucleotide. In certain embodiments, the oligomeric duplex is an antisense agent. In certain embodiments, the first modified oligonucleotide is an antisense RNAi oligonucleotide having a length of 21-23 oligonucleotides. In certain embodiments, the second modified oligonucleotide is a sense RNAi oligonucleotide having a length of 19-21 oligonucleotides.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH₂—; and —O—CH(CH₃)—, and a non-bicyclic sugar moiety, such as a 2′-MOE sugar moiety, a 2′-F sugar moiety, a 2′-OMe sugar moiety, or a 2′-NMA sugar moiety. In certain embodiments, at least one nucleoside of the first modified oligonucleotide and/or the second modified oligonucleotide can comprise an unmodified 2′-deoxyribosyl sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe. In certain embodiments, one or more 2′-F sugar moieties have a conformation other than 2′-β-D-ribosyl. In certain embodiments, one or more 2′-F sugar moieties is in the 2′-β-D-xylosyl conformation.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, hexitol nucleic acid (HNA), fluoro-hexitol nucleic acid (F-HNA), the sugar surrogates of glycol nucleic acid (GNA) and unlocked nucleic acid (UNA). In certain embodiments, at least one nucleoside of the first modified oligonucleotide comprises a sugar surrogate, which can be a GNA.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage. In certain embodiments, the modified internucleoside linkage is a mesyl phosphoramidate internucleoside linkage. In certain embodiments, at least one of the first or second internucleoside linkages from the 5′ end and/or the 3′ end of the first modified oligonucleotide comprises a mesyl phosphoramidate internucleoside linkage. In certain embodiments, at least one of the first or second internucleoside linkages from the 5′ end and/or the 3′ end of the second modified oligonucleotide comprises a mesyl phosphoramidate internucleoside linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester, a phosphorothioate, or a mesyl phosphoramidate internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, each internucleoside linkage of the first modified oligonucleotide and/or the second modified oligonucleotide can be independently selected from a phosphodiester or a mesyl phosphoramidate internucleoside linkage.

In any of the oligomeric duplexes described herein, the internucleoside linkage motif of the first modified oligonucleotide can be ssooooooooooooooooooss, wherein each “s” is a phosphorothioate internucleoside linkage and each “o” is a phosphodiester internucleoside linkage. In any of the oligomeric duplexes described herein, the internucleoside linkage motif of the second modified oligonucleotide can be ssooooooooooooooooss, wherein each “s” is a phosphorothioate internucleoside linkage and each “o” is a phosphodiester internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In some embodiments, the oligomeric duplex has a motif as described in International Publication No. WO 2022/174053.

In any of the oligomeric duplexes described herein, the first modified oligonucleotide and/orthe second modified oligonucleotide can comprise a conjugate group. Preferably, the second modified oligonucleotide comprises a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group is attached to the first modified oligonucleotide at an internal position. In certain embodiments, the conjugate group is attached to the first modified oligonucleotide through a 2′-modification of a furanosyl sugar moiety. In certain embodiments, the conjugate group is attached to the first modified oligonucleotide through a modified internucleoside linkage. In certain embodiments, the conjugate group is attached to the second modified oligonucleotide at the 5′-end of the modified oligonucleotide. In certain embodiments, the conjugate group is attached to the second modified oligonucleotide at the 3′-end of the modified oligonucleotide. In certain embodiments, the conjugate group is attached to the second modified oligonucleotide at an internal position. In certain embodiments, the conjugate group is attached to the second modified oligonucleotide through a 2′-modification of a furanosyl sugar moiety. In certain embodiments, the conjugate group is attached to the second modified oligonucleotide through a modified internucleoside linkage. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group is a bicyclic peptide capable of binding TfR1.

### Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce or inhibit the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, antisense compounds described herein are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid.

In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target RNA is a mature mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

### Complementarity/Mismatches to the Target Nucleic Acid

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### Certain Target Nucleic Acids

Modified oligonucleotides targeting muscle nucleic acids have been described previously that may be useful in conjunction with the provided invention, and in particular modified oligonucleotides may be comprised in compositions of the invention. In certain embodiments, a target nucleic acid is a muscle target nucleic acid. In certain embodiments, the target nucleic acid is selected from ACTC1, ACTN2, ACVR1, ACVR1B, C90RF72, CALR3, CaMK2d, CSRP3, DMD, DMPK, DNM2, DUX4, FBX032, FLNC, FXN, GYS1, HPRT, INHBA, JPH2, KLF15, LDB3, MEDL MEDI3, MEF2D. MSTN, MYBPC3, MYH6, MYH7, MYL2, MYL3, MLCK1, MYOZ2, MYPN, NEXN, NLRP3, PLN, PPPIR3A. PRKAG2, RYR, SOD1, TCAP, TNN, TNNC1, TNNI3, TNNT2, TPM1, TRIM64, or VCL. In certain embodiments, a modified oligonucleotide targeting one or more such target nucleic acids comprises one or more modified oligonucleotides described and set forth in International Patent Publication Nos WO2019/090160, WO2020/028842, WO2020/028841, WO2020/028831, WO2021/142260, WO2021/142227, WO2020/028840, WO2021/142217, WO2021/142331, or WO2021/142269.

In certain embodiments, the muscle target nucleic acid is selected from CaMK2d, NLRP3, PLN, DMD, DMPK, DNM2, DUX4, or HPRT. In certain embodiments, CaMK2d nucleic acid has the sequence set forth SEQ ID NO: 1 (the complement of GENBANK Accession No. NC_000004.12, truncated from nucleosides 113448001 to 113765000) or SEQ ID NO: 2 (GENBANK Accession No. NM_001321571.2). In certain embodiments, NLRP3 nucleic acid has the sequence set forth SEQ ID NO: 3 (GENBANK Accession No. NC_000001.11, truncated from nucleosides 247413001 to 247454000) or SEQ ID NO: 4 (GENBANK Accession No. NM_004895.4). In certain embodiments, PLN nucleic acid has the sequence set forth SEQ ID NO: 5 (GENBANK Accession No. NC_000006.12, truncated from nucleosides 118545001 to 118565000) or SEQ ID NO: 6 (GENBANK Accession No. NM_002667.4). In certain embodiments, DMD nucleic acid has the sequence set forth SEQ ID NO: 7 (The complement of GENBANK NT_011757.15 truncated from nucleotides 28916001 to 31142000). In certain embodiments, DMPK nucleic acid has the sequence set forth SEQ ID NO: 8 (GenBank Accession No. NT_011109.15 truncated from nucleotides 18540696 to 18555106) or SEQ ID NO: 9 (GENBANK Accession No. NM_001081560.1). In certain embodiments, DNM2 nucleic acid has the sequence set forth SEQ ID NO: 10 (GenBank Accession No. NC_000019.10 truncated from nucleosides 10715001 to 10835000) or SEQ ID NO: 11 (GENBANK Accession No. NM_004945.3). In certain embodiments, DUX4 nucleic acid has the sequence set forth SEQ ID NO: 12 (GENBANK Accession No. NC_000004.12, truncated from nucleotides 190171001 to 190187000) or SEQ ID NO: 13 (GENBANK Accession No. NM_001306068.2). In certain embodiments, HPRT1 nucleic acid has the sequence set forth SEQ ID NO: 253 (ENSEMBL ID ENSG00000165704.15, Release 107 (July 2022)).

In certain embodiments, a modified oligonucleotide targeting CaMK2d comprises one or more modified oligonucleotides described and set forth in International Patent Publication Nos. WO2019/165067, WO2021/158810, or WO2022/058386. In certain embodiments, a modified oligonucleotide targeting NLRP3 comprises one or more modified oligonucleotides described and set forth in International Patent Publication No. WO2022178146A1, which is incorporated herein by reference. In certain embodiments, a modified oligonucleotide targeting PLN comprises one or more modified oligonucleotides described and set forth in International Patent Publication No. WO2022/173976, which is incorporated herein by reference, or International Patent Publication No. WO2001/16312. In certain embodiments, a modified oligonucleotide targeting DMD comprises one or more oligonucleotides described and set forth in WO2018/014042, which is incorporated herein by reference, or International Patent Publication Nos. WO2022/020107, WO2021/025899, WO2021/003573, WO2021/142307, WO2020/257489, WO2020/219820, WO2020/214763, WO2020/198268, WO2020/089325, WO2020/028832, WO2019/200185, WO2019/090160, WO2019/060775, WO2019/014772, WO2018/129384, WO2018/067973, WO2018/055577, WO2018/014043, WO2018/014042, WO2018/007475, WO2018/005805, WO2017/210647, WO2017/192679, WO2017/047707, WO2015/137409, WO2014/153220, WO2013/112053, WO2013/100190, WO2012/029986, WO2011/057350, WO2010/123369, WO2010/048586, WO2009/054725, WO2007/135105, WO2006/000057, WO2004/083446, WO2004/048570, or WO2002/024906. In certain embodiments, a modified oligonucleotide targeting DMPK comprises one or more oligonucleotides described and set forth in International Patent Publication Nos. WO 2012/012443, WO2012/012467, WO2015/021457, which are incorporated herein by reference, or International Patent Publication Nos. WO2022/147209, WO2022/026152, WO2021142234, WO2021/076856, WO2020/028861, WO2019/113393, WO2006/006948, WO 2005/116204, WO2018/002812, WO2018/078131, or WO2018/078134. In certain embodiments, a modified oligonucleotide targeting DNM2 comprises one or more oligonucleotides described and set forth in International Patent Publication No. WO2019/140452, which is incorporated herein by reference, or International Patent Publication Nos. WO2020/028844, WO2015/055859, or WO2016/170162. In certain embodiments, a modified oligonucleotide targeting DUX4 comprises one or more oligonucleotides described and set forth in International Patent Publication Nos. WO2016/115490, WO2022/159712, which are incorporated herein by reference, or US Patent Publication No. US2021220479, or International Patent Publication Nos. WO2022/147207, WO2022/020106, WO2021/142275, WO2020/028840, WO2020/203880, WO2020/028864, WO2019/060432, WO 2017/050836, WO2016/115490, or WO2012/024535.

### Certain Pharmaceutical Compositions

In certain embodiments, pharmaceutical compositions described herein comprise one or more oligomeric compounds. In certain embodiments, the one or more oligomeric compounds each comprise a modified oligonucleotide. In certain embodiments, the pharmaceutical composition comprises a pharmaceutically acceptable diluent or carrier. In certain embodiments, a pharmaceutical composition comprises a sterile saline solution and one or more oligomeric compounds. In certain embodiments, a pharmaceutical composition consists or consists essentially of a sterile saline solution and one or more oligomeric compounds. In certain embodiments, the sterile saline is pharmaceutical grade saline. In certain embodiments, a pharmaceutical composition comprises one or more oligomeric compounds and sterile water. In certain embodiments, a pharmaceutical composition consists or consists essentially of one or more oligomeric compounds and sterile water. In certain embodiments, the sterile water is pharmaceutical grade water. In certain embodiments, the pharmaceutically acceptable diluent or carrier is distilled water for injection. In certain embodiments, a pharmaceutical composition comprises one or more oligomeric compound and phosphate-buffered saline (PBS). In certain embodiments, a pharmaceutical composition consists or consists essentially of one or more oligomeric compounds and PBS. In certain embodiments, the sterile PBS is pharmaceutical grade PBS. In certain embodiments, a pharmaceutical composition comprises one or more oligomeric compound and artificial cerebrospinal fluid. In certain embodiments, the sterile PBS is pharmaceutical grade PBS. In certain embodiments, a pharmaceutical composition consists or consists essentially of artificial cerebrospinal fluid. In certain embodiments, the artificial cerebrospinal fluid is pharmaceutical grade.

In certain embodiments, pharmaceutical compositions comprise one or more oligomeric compounds disclosed herein and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound disclosed herein encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise a conjugate moiety attached to an oligonucleotide, wherein the conjugate moiety is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of an oligomeric agent to a particular cell or tissue. In certain embodiments, a lipid moiety is selected to increase distribution of an oligomeric agent to fat tissue. In certain embodiments, a lipid moiety is selected to increase distribution of an oligomeric agent to muscle tissue.

In certain embodiments, pharmaceutical compositions disclosed herein comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver oligomeric compounds described herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions disclosed herein are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

Under certain conditions, certain compounds disclosed herein act as acids. Although such compounds may be drawn or described in protonated (free acid) form, in ionized (anion) form, or ionized and in association with a cation (salt) form, aqueous solutions of such compounds exist in equilibrium among such forms. For example, a phosphate linkage of an oligonucleotide in aqueous solution exists in equilibrium among free acid, anion, and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or salts thereof” expressly includes all such forms that may be fully or partially protonated/de-protonated/in association with a cation. In certain instances, one or more specific cation is identified.

In certain embodiments, oligomeric compounds disclosed herein are in aqueous solution with sodium. In certain embodiments, oligomeric compounds are in aqueous solution with potassium. In certain embodiments, oligomeric compounds are in PBS. In certain embodiments, oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide, excluding the mass of any conjugate group. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid. For example, where a modified oligonucleotide or an oligomeric compound is in solution comprising sodium (e.g., saline), the modified oligonucleotide or oligomeric compound may be partially or fully de-protonated and in association with Na+ ions. However, the mass of the protons is nevertheless counted toward the weight of the dose, and the mass of the Na+ ions is not counted toward the weight of the dose. Furthermore, the mass of a conjugate group or bicycle ligand is not included when calculating the weight of a dose as described herein; that is, the dose relates only to the oligonucleotide or oligomeric duplex. Thus, for example, a dose, or dosage unit, of 3.5 mg of Compound No. 486178 or Compound No. 1590463-BCY17868, equals the number of molecules of the fully protonated oligonucleotide portion of the molecule that weighs 3.5 mg. This is equivalent to 3.7 mg of solvent-free, sodium acetate-free, anhydrous sodiated Compound No. 486178; and it is equivalent to 4.7 mg of the conjugated compound 1590463-BCY17868.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: Design and Synthesis of Bicycle Ligands

Polypeptides were synthesized on Rink amide resin using standard Fmoc (9-fluorenylmethyloxycarbonyl) solid-phase peptide synthesis, either by manual coupling (for large scale) or using a Biotage SyroII automated peptide synthesizer (for small scale). Following TFA-based cleavage from the resin, peptides were precipitated with diethyl ether and dissolved in 50:50 acetonitrile/water. The crude peptides (at −1 mM concentration) were then cyclized with 1.3 equiv. of the molecular scaffold, 1,1′,1″-(1,3,5-triazinane-1,3,5-triyl)tris(2-bromoethanone) (TATB), using ammonium bicarbonate (100 mM) as abase. Completion of cyclization was determined by matrix-assisted laser desorption ionization time-of-flight (MALDI-TOF) or LC-MS. Once complete, the cyclization reaction was quenched using N-acetyl cysteine (10 equiv. with respect to the peptide), and the solutions were lyophilized. The residue was dissolved in an appropriate solvent and purified by RP-HPLC. Peptide fractions of sufficient purity and the correct molecular weight (verified by either MALDI-TOF and HPLC or LC-MS) were pooled and lyophilized. Concentrations were determined by UV absorption using the extinction coefficient at 280 nm, which was based on Trp/Tyr content. All amino acids, unless noted otherwise, are in the L-configurations. Each C-terminus is amidated.

TABLE 2
Bicycle ligands

| Bicycle Ligand | | N-terminal | SEQ ID |
|---|---|---|---|
| ID | Sequence (N to C) | modification | NO: |
| BCY17868 | CSPDAHLGCISYC[K(N₃)] | acetyl | 71 |
| BCY17869 | CSPDAHLGCISYC | azidopropyl | 26 |
| BCY17870 | NWNCSPDAHLGCISYC[K(N₃)] | none | 69 |
| BCY17871 | NWNCSPDAHLGCISYC[K(N₃)] | acetyl | 69 |
| BCY17872 | NWNCSPDAHLGCISYC | azidopropyl | 53 |
| BCY17873 | CSPDAHLGCISYCEPW[K(N₃)] | acetyl | 70 |
| BCY17874 | CSPDAHLGCISYCEPW | azidopropyl | 52 |
| BCY17875 | CP[HyP]DAYLGCISYC[K(N₃)] | acetyl | 94 |
| BCY17876 | CP[HyP]DAYLGCISYC | azidopropyl | 93 |
| BCY17877 | CS[HyP]DAHLGCISYC[K(N₃)] | acetyl | 97 |
| BCY17878 | CS[HyP]DAHLGCISYC | azidopropyl | 95 |
| BCY17879 | CS[Aze]DAHLGCISYC[K(N₃)] | acetyl | 129 |
| BCY17880 | CS[Aze]DAHLGCISYC | azidopropyl | 128 |
| BCY17882 | N[dY]NCSPDAHLGCISYC[K(N₃)] | acetyl | 72 |
| BCY17890 | CSPDAHLGCISYCE[dP]W[K(N₃)] | acetyl | 73 |
| BCY17892 | CSPDAHLGCISYCE[Aze]W[K(N₃)] | acetyl | 74 |
| BCY17894 | CSPDAHLGCISYCE[Pip]W[K(N₃)] | acetyl | 75 |
| BCY17896 | CP[HyP]DAYLGC[tBuGly]SYC[K(N₃)] | acetyl | 90 |
| BCY17899 | NWNCP[HyP]DAYLGC[tBuGly]SYC[K(N₃)] | acetyl | 91 |
| BCY17901 | CP[HyP]DAYLGC[tBuGly]SYCEPW[K(N₃)] | acetyl | 92 |
| BCY17903 | C[K(N₃)]PDAHLGCISYC | acetyl | 150 |
| BCY17904 | CS[K(N₃)]DAHLGCISYC | acetyl | 151 |
| BCY17905 | CSPD[K(N₃)]HLGCISYC | acetyl | 152 |
| BCY17906 | CSPDAHLGCISYC[K(N₃)(PYA-Maleimide)] | acetyl | 76 |

dY represents _{D}-tyrosine; [HyP] represents 4-trans-hydroxy-L-proline; [Aze] represents azetidine; [tBuGly] represents t-butyl glycine; [K(N₃)] represents 6-azido lysine; [Pip] represents pipecolic acid; and [K(N₃)(PYA-Maleimide)] represents a modified lysine having the following structure:

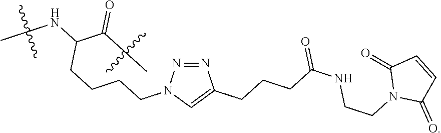 [SMILES: *NC(CCCCn1cc(CCCC(=O)NCCN2C(=O)C=CC2=O)nn1)C(*)=O]

### Example 2: Design of Modified Oligonucleotides Complementary to Mouse DMPK

Modified oligonucleotides complementary to mouse DMPK were designed (as indicated in the table below) and synthesized. Selected compounds in the table below have modifications on the 5′ end to allow conjugation to a bicycle ligand. Compound No. 486178 was previously disclosed in WO 2014/120861.

TABLE 3
Design of modified oligonucleotides complementary to mouse DMPK

| | | SEQ |
|---|---|---|
| Compound | | ID |
| No. | Chemistry Notation (5′ to 3′) | NO. |
| 486178 | Aₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | 167 |
| 1590463 | [BCN][nC6o]ₒAₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | 176 |
| 1614439 | [sC6o]ₒAₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | 179 |
| 1602000 | [PEG1alkyne][nC6o]ₒAₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | 178 |
| 1609732 | [BCN][nC6o]ₒ[T_{do}^{m}C_{do}A_{d}]ₒAₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | 168 |
| 1468770 | [maleimidC3oyl][nC6o]ₒAₖₛ^{m}CₖₛAₖₛA_{ds}T_{ds}A_{ds}A_{ds}A_{ds}T_{ds}A_{ds}^{m}C_{ds}^{m}C_{ds}G_{ds}AₖₛGₖₛGₖ | 175 |

A subscript “k” represents a cEt nucleoside, a subscript “d” represents a stereo-standard DNA nucleoside, a subscript “s” indicates a phosphorothioate internucleoside linkage, a subscript “o” indicates a phosphodiester internucleoside linkage, a superscript “m” before a C represents a 5-methylcytosine, a “[nC6o]” indicates a 6-aminohexanol linker, a “[BCN]” indicates a (bicyclo[6.1.0]nonyne)-formyl linker, a “[sC6o]” indicates a 6-mercaptohexanol liner, a
“[PEG1alkyne]” indicates a propargyl-PEG1-acid, a “[T_{do}^{m}C_{do}A_{d}]” indicates a TCA trinucleotide linker, and a “[maleimidC3oyl]” indicates a maleimido propionyl linker having structure:

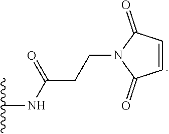 [SMILES: *NC(=O)CCN1C(=O)C=CC1=O]

A modified oligonucleotide complementary to 486178 (described herein above) was designed (as described in the table below) and synthesized. The compound in the table below has a modification on the 5′ end to allow conjugation to a bicycle ligand.

TABLE 4
Design of a modified oligonucleotide with linkers for conjugation to CD71
bicycle ligands, complementary to 486178.

| Compound | | SEQ ID |
|---|---|---|
| No. | Chemistry Notation (5′ to 3′) | NO. |
| 1614438 | [BCN][nC6o]ₒ^{m}Cₖₒ^{m}CₖₒTₖₒ^{m}C_{do}G_{do}G_{do}T_{do}A_{do}T_{do}T_{do}T_{do}A_{do}T_{do}TₖₒGₖₒTₖ | 169 |

A subscript “k” represents a cEt nucleoside, a subscript “d” represents a stereo-standard DNA nucleoside, a subscript “o” indicates a phosphodiester internucleoside linkage, a superscript “m” before a C represents a 5-methylcytosine, a “[nC60]” indicates a 6-aminohexanol linker, and a “[BCN]” indicates a (bicyclo[6.1.0]nonyne)-formyl linker.

Modified oligonucleotides complementary to mouse MALAT were designed (as indicated in the table below) and synthesized. The compound in the table below has a modification on the 3′ end to allow conjugation to a bicycle ligand.

TABLE 5
Design of modified oligonucleotides complementary to mouse MALAT

| Compound | | SEQ |
|---|---|---|
| No. | Chemistry Notation (5′ to 3′) | ID NO. |
| 1598988 | [5Cy3cHex]ₛGₑₒ^{m}CₑₒAₑₒTₑₒTₑₒ^{m}CₑₒTₑₒAₑₒAₑₒTₑₒAₑₒGₑₒ^{m}CₑₒAₑₒGₑₒ^{m}Cₑₒ[3nC7][BCN] | 177 |
| 1591118 | [5Cy3cHex]ₛGₖₛ^{m}CₖₛAₖₛT_{ds}T_{ds}^{m}C_{ds}T_{ds}A_{ds}A_{ds}T_{ds}A_{ds}G_{ds}^{m}C_{ds}AₖₛGₖₛ^{m}Cₖₒ[3nC7][BCN] | 170 |

A subscript “e” represents a 2′-MOE modified nucleoside, a subscript “s” indicates a phosphorothioate internucleoside linkage, a subscript “o” indicates a phosphodiester internucleoside linkage, a superscript “m” before a C represents a 5-methylcytosine, a “[BCN]” indicates a (bicyclo[6.1.0]nonyne)-formyl linker, a “[5Cy3cHex]” indicates a 5′-Cy3-cyclohexane moiety (GenePharma 11-4100), and a “[3nC7]” represents a 3′-C7 amino modifier having the following structure:

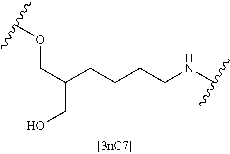 [SMILES: *NCCCCC(CO)CO*]

### Example 3: Design of RNAi Compounds Targeted to HPRT1

Antisense strand modified oligonucleotides complementary to mouse HPRT were designed and synthesized as indicated in the table below.

TABLE 6
Design of antisense strand modified oligonucleotides targeted to human/mouse HPRT1

| | | | SEQ |
|---|---|---|---|
| Compound | | | ID |
| No. | Chemistry Notation (5′ to 3′) | Target | NO. |
| 1453015 | A_{ys}U_{fs}A_{yo}A_{yo}A_{yo}A_{fo}U_{yo}C_{yo}U_{yo}A_{yo}C_{yo}A_{yo}G_{yo}U_{fo}C_{yo}A_{fo}U_{yo}A_{yo}G_{yo}G_{yo}A_{ys}A_{ys}U_{y} | mouse | 171 |
| | | HPRT | |

In the table above, a subscript “f” represents a 2′-F modified nucleoside, a subscript “y” represents a 2′-OMe modified nucleoside, a subscript “s” represents a phosphorothioate internucleoside linkage, and a subscript “o” represents a phosphodiester internucleoside linkage.

The sense oligonucleotide is complementary to the first of the 21 nucleosides of the antisense oligonucleotide (from 5′ to 3′) wherein the last two 3′-nucleosides of the antisense oligonucleotides are not paired with the sense oligonucleotide (are overhanging nucleosides).

TABLE 7
Design of sense strand modified oligonucleotides targeted to human/mouse HPRT1
containing C16-modified nucleosides

| | | SEQ |
|---|---|---|
| Compound | | ID |
| No. | Chemistry Notation (5′ to 3′) | NO. |
| 1550983 | U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{yo}A_{yo}U_{yo}[3nC7] | 181 |
| | [BCN] | |
| 1547771 | U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{yo}A_{yo}U_{yo}[3nC7] | 172 |
| | [maleimidC3oyl] | |

In the table above, a subscript “f” represents a 2′-F modified nucleoside, a subscript “y” represents a 2′-OMe modified nucleoside, a subscript “s” represents a phosphorothioate internucleoside linkage, a subscript “o” represents a phosphodiester internucleoside linkage, a “[BCN]” indicates a (bicyclo[6.1.0]nonyne)-formyl linker, a “[3nC7]” represents a 3′-C7 amino modifier, and a “[maleimidC3oyl]” indicates a maleimido propionyl linker.

TABLE 8
Design of RNAi compounds targeted to human/mouse HPRT1

| Duplex | Antisense Strand | Sense Strand |
|---|---|---|
| Compound No. | Compound No. | Compound No. |
| 1453015:1550983 | 1453015 | 1550983 |
| 1453015:1547771 | 1453015 | 1547771 |

### Example 4: In Vitro Binding Assay for Binding of Oligomeric Compounds Comprising CD71 Bicycle Ligands to Human Transferrin Receptor, CD71

A nanoBRET assay was developed to obtain inhibition constant Kᵢ of modified oligonucleotides conjugated to CD71 bicycle ligands and RNAi compounds conjugated to CD71 bicycle ligands. The use of the nanoBRET assay for the binding of oligonucleotides to proteins has been previously described (see, e.g., Vickers and Crooke, PloS One, 2016, 11(8):e061930).

An hCD71-Nluc fusion protein was constructed by linking NanoLuc (ProMega) through its N-terminal Val to the C-terminal F760 residue of h-CD71, using a GGGSGGSSG flexible linker. The fluorescently-labeled (Cy3) modified oligonucleotide 1598988 was conjugated to the bicycle ligand BCY17871 through a strain-promoted azide-alkyne cycloaddition (SPAAC) reaction.

Crude membrane fractions from HEK293 cells stably expressing hCD71-Nluc were resuspended in PBS and 100 μL were dispensed into white 96-well assay plates (ThermoFisher Scientific, Cat #136101), an amount corresponding to 10,000 cells per well. Membranes were treated with 11.1 μL of BCY17871-1598988 at serially diluted final concentrations of 1000-0.006 nM. The mixtures were incubated for 3 hours at room temperature to reach equilibrium. To initiate BRET, 12.4 μL of 100 μM of the Nluc substrate furimazine was added to each well, and the mixtures were incubated for 5-30 minutes. The assay plate was read on a Promega GlowMax Discover plate reader at wavelengths of 450 nm and 600 nm, and the ratio of emissions at wavelengths 450/600 were used to yield % BRET efficiency. Data was subjected to non-linear regression, then fitted to a single site binding hyperbolic function. The dissociation constant K_{D} of BC17871-1598988 was determined to be 57-58 nM.

In order to evaluate modified oligonucleotides conjugated to CD71 bicycle ligands and RNAi compounds conjugated to CD71 bicycle ligands, the assay was modified as follows. 100 μL of crude membrane fractions from stably transfected hCD71-Nluc HEK293 cells were dispensed into white 96-well assay plates (ThermoFisher Scientific, Cat #136101). BCY17871-1598988 was used as a tracer compound and added to each well at a final concentration of 60 nM. Modified oligonucleotides were conjugated to the bicycle ligands through a SPAAC reaction These were then added at a range of concentrations in triplicate assays points, and the mixtures were incubated for 3 hours at room temperature. BRET was initiated with the addition of furimazine and the assay was completed as described above. Inhibition constants (Kᵢ) were obtained by fitting % BRET efficiency values to a competitive inhibition model, using the K_{D} value estimated for BCY17871-1598988 obtained in the same run. Values are presented as the average of triplicate data in the tables below. Each experiment is presented in a separate table.

The bicycle ligands were attached to the modified oligonucleotides through a BCN linker. Compound 1590463-BCY17901 is shown below as an example:

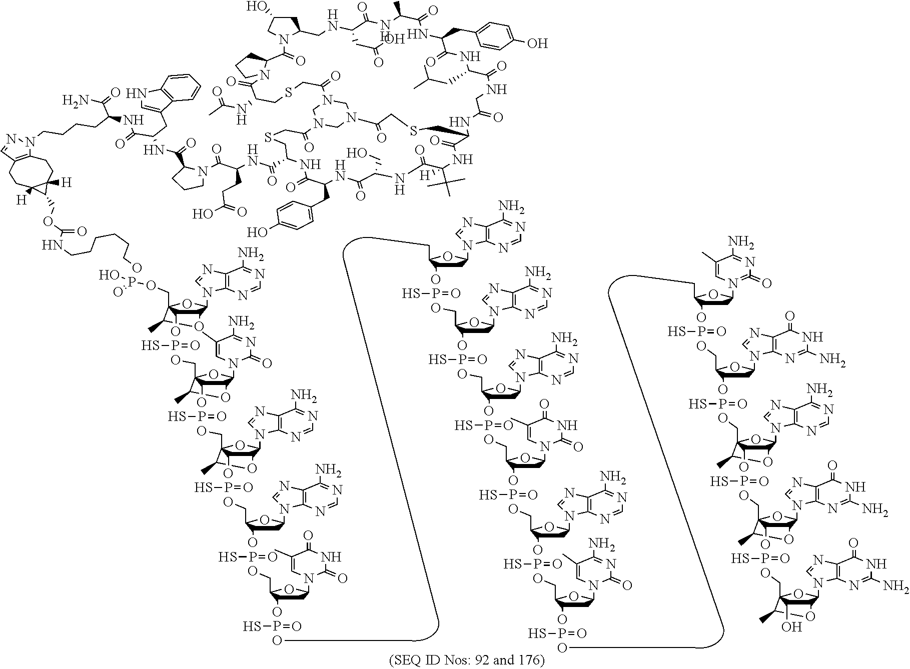

TABLE 9
Inhibition constants for modified oligonucleotides
conjugated to CD71 bicycle ligands, by nanoBRET

| Compound Number | Bicycle Ligand | Kᵢ (nM) |
|---|---|---|
| 1590463 | BCY17868 | 20 |
| 1590463 | BCY17869 | 55 |
| 1590463 | BCY17870 | 7 |
| 1590463 | BCY17871 | 6 |
| 1590463 | BCY17872 | 21 |
| 1590463 | BCY17873 | 6 |
| 1590463 | BCY17874 | 10 |
| 1590463 | BCY17875 | 4 |
| 1590463 | BCY17876 | 29 |
| 1590463 | BCY17877 | 18 |
| 1590463 | BCY17878 | 74 |
| 1590463 | BCY17879 | 25 |
| 1590463 | BCY17880 | 102 |

TABLE 10
Inhibition constants for modified oligonucleotides
conjugated to CD71 bicycle ligands, by nanoBRET

| | Bicycle | |
|---|---|---|
| Compound Number | Ligand | Kᵢ (nM) |
| 1590463 | BCY17882 | 23 |
| 1590463 | BCY17890 | 17 |
| 1590463 | BCY17892 | 11 |
| 1590463 | BCY17894 | 8 |
| 1590463 | BCY17896 | 2 |
| 1590463 | BCY17899 | 4 |
| 1590463 | BCY17901 | 1 |
| 1590463 | BCY17903 | 125 |
| 1590463 | BCY17904 | 31 |
| 1590463 | BCY17905 | 148 |
| 1590463 | BCY17873 | 7 |

TABLE 11
Inhibition constants for modified oligonucleotides
conjugated to CD71 bicycle ligands, by nanoBRET

| | Bicycle | |
|---|---|---|
| Compound Number | Ligand | Kᵢ (nM) |
| 1590463 | BCY17882 | 3 |
| 1590463 | BCY17890 | 2 |
| 1590463 | BCY17892 | 1 |
| 1590463 | BCY17894 | 1 |
| 1590463 | BCY17896 | 1 |
| 1590463 | BCY17899 | 1 |
| 1590463 | BCY17901 | 0.3 |
| 1590463 | BCY17903 | 24 |
| 1590463 | BCY17904 | 5 |
| 1590463 | BCY17905 | 16 |
| 1614439 | BCY17906 | 2 |
| 1590463 | BCY17873 | 1 |
| 1602000 | BCY17873 | 1 |
| 1609732 | BCY17873 | 1 |

TABLE 12
Inhibition constants for modified oligonucleotides
conjugated to CD71 bicycle ligands, by nanoBRET

| | Bicycle | |
|---|---|---|
| Compound Number | Ligand | Kᵢ (nM) |
| 1590463 | BCY17882 | 55 |
| 1590463 | BCY17890 | 20 |
| 1590463 | BCY17892 | 11 |
| 1590463 | BCY17894 | 10 |
| 1590463 | BCY17896 | 2 |
| 1590463 | BCY17899 | 4 |
| 1590463 | BCY17901 | 1 |
| 1590463 | BCY17904 | 60 |
| 1614439 | BCY17906 | 23 |
| 1590463 | BCY17873 | 10 |
| 1602000 | BCY17873 | 36 |
| 1609732 | BCY17873 | 10 |

TABLE 13
Inhibition constants for modified oligonucleotides
conjugated to CD71 bicycle ligands, by nanoBRET

| | Bicycle | |
|---|---|---|
| Compound Number | Ligand | Kᵢ (nM) |
| 1590463 | BCY17873 | 4 |
| 1590463 | BCY19405 | 3 |
| 1590463 | BCY19406 | 2 |
| 1590463 | BCY19407 | 2 |
| 1590463 | BCY19409 | 1 |

TABLE 14
Inhibition constants for modified oligonucleotides
conjugated to CD71 bicycle ligands, by nanoBRET

| | Bicycle | |
|---|---|---|
| Compound Number | Ligand | Kᵢ (nM) |
| 1598988 | BCY17896 | 9.9 |
| 1591118 | BCY17896 | 3.1 |
| 1598988 | BCY17901 | 4.4 |
| 1591118 | BCY17901 | 1.4 |

A duplex of modified oligonucleotides was generated by mixing Compound Nos. 486178 and 1614438 to generate Compound No. 486178:1614438.

TABLE 15
Inhibition constants for modified oligonucleotide duplexes
conjugated to CD71 bicycle ligands, by nanoBRET

| | Bicycle | |
|---|---|---|
| Compound Number | Ligand | Kᵢ (nM) |
| 486178:1614438 | BCY17873 | 95 |

TABLE 16
Inhibition constants for RNAi compounds conjugated
to CD71 bicycle ligands, by nanoBRET

| | Bicycle | |
|---|---|---|
| Compound Number | Ligand | Kᵢ (nM) |
| 1453015:1550983 | BCY17873 | 9 |

TABLE 17
Inhibition constants for RNAi compounds conjugated
to CD71 bicycle ligands, by nanoBRET

| | Bicycle | |
|---|---|---|
| Compound Number | Ligand | Kᵢ (nM) |
| 1453015:1550983 | BCY17873 | 38 |

### Example 5: Binding Affinity of Modified Oligonucleotides Conjugated to CD71 Bicycle Ligands

The binding affinity for each of the following modified oligonucleotides conjugated to CD71 bicycle ligands was tested on a Biacore X100 surface plasmon resonance (SPR) instrument. 200 units of modified oligonucleotides conjugated to CD71 bicycle ligand were immobilized on a streptavidin chip by injecting a 20 nM solution of 5′-Biotin-labeled DNA (5′-Biotin-TEG-DNA complement to modified oligonucleotide) duplexed with modified oligonucleotides conjugated to CD71 bicycle ligand in HBS—P (10 mM HEPES, pH 7.4, 150 mM NaCl, 0.0005% Surfactant P20) running buffer. CD71 in running buffer was then injected onto the modified oligonucleotides conjugated to CD71 bicycle ligand duplex-immobilized streptavidin chip at 25° C., at increasing concentrations of 6.25 nM, 12.5 nM, 25 nM, 50 nM, and 100 nM. Kinetic and equilibrium binding analysis was performed using Biacore X100 Evaluation Software, applying 1:1 binding fit. Binding affinity is expressed as equilibrium dissociation constant (K_{D}) in the table below.

TABLE 18
Binding affinity of modified oligonucleotides
conjugated to CD71 bicycle ligands, SPR

| Compound Number | Bicycle Ligand | K_{D} (nM) |
|---|---|---|
| 1590463 | BCY17868 | 0.9 |
| 1590463 | BCY17869 | 14.5 |
| 1590463 | BCY17870 | 0.1 |
| 1590463 | BCY17871 | 2.4 |
| 1590463 | BCY17872 | 77.1 |
| 1590463 | BCY17873 | 12 |
| 1590463 | BCY17874 | 32 |
| 1590463 | BCY17875 | 4.5 |
| 1590463 | BCY17876 | 95.4 |
| 1590463 | BCY17877 | 12.5 |
| 1590463 | BCY17878 | 126 |
| 1590463 | BCY17879 | 31.2 |
| 1590463 | BCY17880 | 8.2 |

### Example 6: Activity and Tolerability of Modified Oligonucleotides Conjugated to CD71 Bicycle Ligands in hTFR^{KI/+} Knock in Mice

Human transferrin receptor (hTFR)/CD71 knock-in mice used in these studies have the coding region of mouse exon 2 as well as the splice donor-site of mouse intron 2 replaced with the human TFR open reading frame according to NCBI transcript NM_001128148.2. Humanization of the transferrin receptor gene was done via CRISPR/Cas-9-mediated gene editing, allowing for generation of a model with constitutive expression of humanized transferrin receptor gene. Targeting strategy was based on NCBI transcripts NM_011638.4 (mouse) and NM_001128148.2 (human). A plasmid allowing expression of Cas9 mRNA, specific gRNA, and the puromycin resistance cassette; and a plasmid containing the homology regions of the mouse transferrin receptor gene, an FRT site, and the replaced human region were co-transfected into the Taconic Biosciences C57BL/6N Tac ES cell line. The humanized mice are called hTFR^{KI/+} knock-in mice herein. They express one copy of the mouse TFR gene and one copy of the humanized TFR gene under the control of the endogenous mouse promoter.

Activity and tolerability of modified oligonucleotides conjugated to CD71 bicycle ligands was tested in in hTFR^{KI/+} knock in mice. In addition, activity of Compound No. 1468770 (described herein above) conjugated to the Fab′ fragments of OKT9 antibody (BioXCell, catalog number: BE0023) that targets human CD71 was tested.

### Treatment

hTFR^{KI/+} mice were divided into groups of 3 mice each. Each mouse received an intravenous administration of conjugated modified oligonucleotide for a total of 3 doses (on Days 1, 8, and 15) at doses indicated in the table below. A group of 3 mice received an intravenous administration of unconjugated modified oligonucleotide, Compound No. 486178, for a total of 3 doses (on Days 1, 8, and 15) at doses indicated in the table below. A group of 4 mice received PBS as a negative control.

### RNA Analysis

The mice were sacrificed one week post final administration (on day 22), and RNA was extracted from various muscle tissues (including quadriceps (Quad), tibialis anterior (TA), diaphragm, triceps, heart, gastrocnemius (gastroc)), aorta, sciatic nerve, and liver tissue for quantitative real time RTPCR analysis to measure amount of mouse DMPK RNA using mouse primer probe set RTS3181 (forward sequence GACATATGCCAAGATTGTGCACTAC, designated herein as SEQ ID NO: 16; reverse sequence CACGAATGAGGTCCTGAGCTT, designated herein as SEQ ID NO: 17; probe sequence AACACTTGTCGCTGCCGCTGGC, designated herein as SEQ ID NO: 18). Results are presented as percent mouse DMPK RNA relative to PBS control, normalized to mouse GAPDH (% ocontrol).

TABLE 19
Reduction of mouse DMPK RNA in hTFR^{KI/+} knock in mice
with modified oligonucleotides conjugated to CD71 bicycle ligands

| | | | mouse DMPK RNA (% control) | | | | | | | | |
|---|---|---|---|---|---|---|---|---|---|---|---|
| Compound | | Dose | | | | | | | | | Sciatic |
| No. | Conjugate | (mg/kg) | Quad | TA | Diaphragm | Heart | Gastroc | Triceps | Aorta | Liver | Nerve |
| 486178 | None | 35 | 25 | 36 | 30 | 53 | 24 | 31 | 44 | 10 | 50 |
| 1468770 | OKT9 | 3.5 | 19 | 23 | 24 | 39 | 16 | 28 | 61 | 11 | 72 |
| 1590463 | BCY17868 | 3.5 | 47 | 58 | 46 | 76 | 44 | 56 | 60 | 28 | 71 |
| 1590463 | BCY17869 | 3.5 | 48 | 64 | 51 | 74 | 46 | 58 | 69 | 25 | 65 |
| 1590463 | BCY17877 | 3.5 | 45 | 54 | 54 | 78 | 42 | 54 | 103 | 34 | 72 |
| 1590463 | BCY17878 | 3.5 | 52 | 57 | 55 | 81 | 46 | 56 | 93 | 31 | 87 |
| 1590463 | BCY17879 | 3.5 | 46 | 55 | 50 | 79 | 43 | 55 | 78 | 29 | 76 |
| 1590463 | BCY17880 | 3.5 | 49 | 58 | 60 | 82 | 46 | 53 | 80 | 30 | 71 |
| 1590463 | BCY17875 | 3.5 | 34 | 39 | 44 | 72 | 30 | 40 | 61 | 30 | 71 |
| 1590463 | BCY17876 | 3.5 | 54 | 61 | 59 | 84 | 48 | 57 | 59 | 27 | 81 |
| 1590463 | BCY17870 | 3.5 | 35 | 43 | 44 | 70 | 32 | 40 | 67 | 29 | 72 |
| 1590463 | BCY17872 | 3.5 | 36 | 44 | 43 | 73 | 35 | 44 | 64 | 26 | 70 |
| 1590463 | BCY17871 | 3.5 | 40 | 51 | 44 | 71 | 37 | 49 | 61 | 27 | 79 |
| 1590463 | BCY17873 | 3.5 | 32 | 40 | 39 | 70 | 31 | 38 | 62 | 27 | 73 |
| 1590463 | BCY17874 | 3.5 | 36 | 48 | 45 | 72 | 36 | 43 | 69 | 29 | 72 |

### Plasma Chemistry Markers

To evaluate the effect of modified oligonucleotides on liver and kidney function, plasma levels of albumin (ALB), alanine aminotransferase (ALT), aspartate aminotransferase (AST), blood urea nitrogen (BUN), total bilirubin (TBIL), total Protein (PROT), Creatine (CREAT), and Creatine Kinase (CK) were measured on the day the mice were sacrificed (day 22) using an automated clinical chemistry analyzer (Hitachi Olympus AU400c, Melville, NY). The results were averaged for each group of mice and are presented in the tables below. Oligomeric compounds that caused changes in the levels of any of the liver or kidney function markers outside the expected range for modified oligonucleotides were excluded from further studies.

TABLE 20
Plasma chemistry markers in hTFR^{KI/+} knock in mice

| | | | Plasma clinical chemistry | | | | | | | |
|---|---|---|---|---|---|---|---|---|---|---|
| Com- | | Dose | | | | TBIL | BUN | | | |
| pound | | (mg/ | ALB | ALT | AST | (mg/ | (mg/ | PROT | CREAT | CK |
| No. | Conjugate | kg) | (g/dL) | (U/L) | (U/L) | dL) | dL) | (g/dL) | (mg/dL) | (U/L) |
| PBS | None | 0 | 2.95‡ | 30‡ | 124‡ | 0.19‡ | 23‡ | 5.03‡ | 0.13‡ | 409‡ |
| 486178 | None | 35 | 2.95 | 44 | 199 | 0.29 | 20 | 5.03 | 0.15 | 671 |
| 1468770 | OKT9 | 3.5 | 3.18 | 28 | 113 | 0.19 | 21 | 5.00 | 0.11 | 372 |
| 1590463 | BCY17868 | 3.5 | 3.04 | 51 | 172 | 0.16 | 19 | 4.97 | 0.14 | 571 |
| 1590463 | BCY17869 | 3.5 | 3.01 | 56 | 143 | 0.21 | 17 | 4.90 | 0.11 | 350 |
| 1590463 | BCY17870 | 3.5 | 3.18 | 40 | 132 | 0.20 | 20 | 5.20 | 0.17 | 374 |
| 1590463 | BCY17871 | 3.5 | 3.20 | 45 | 167 | 0.24 | 17 | 5.33 | 0.16 | 505 |
| 1590463 | BCY17872 | 3.5 | 3.21 | 42 | 116 | 0.17 | 18 | 5.23 | 0.15 | 297 |
| 1590463 | BCY17873 | 3.5 | 3.11 | 68 | 231 | 0.20 | 19 | 5.13 | 0.12 | 723 |
| 1590463 | BCY17874 | 3.5 | 3.31 | 28 | 107 | 0.13 | 17 | 5.33 | 0.15 | 395 |
| 1590463 | BCY17875 | 3.5 | 2.91 | 50 | 147 | 0.13 | 18 | 4.60 | 0.11 | 486 |
| 1590463 | BCY17876 | 3.5 | 2.86 | 25 | 133 | 0.13 | 15 | 4.57 | 0.13 | 463 |
| 1590463 | BCY17877 | 3.5 | 3.19 | 22 | 85 | 0.18 | 19 | 5.27 | 0.12 | 163 |
| 1590463 | BCY17878 | 3.5 | 3.20 | 31 | 160 | 0.14 | 19 | 5.23 | 0.14 | 531 |
| 1590463 | BCY17879 | 3.5 | 2.94 | 44 | 176 | 0.12 | 16 | 4.83 | 0.12 | 554 |
| 1590463 | BCY17880 | 3.5 | 3.15 | 34 | 268 | 0.20 | 17 | 4.97 | 0.13 | 1080 |

‡indicates that there were fewer than 4 samples available

### Hematology Assays

Blood obtained from mice on the day the mice were sacrificed (day 22) were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that ofmonocytes (MON), neutrophils (NEU), lymphocytes (LYM), eosinophils (EOS), basophils (BAS), reticulocytes, and platelets were evaluated. The results are presented in the tables below. Oligomeric compounds that caused changes in the blood cell count outside the expected range were excluded in further studies.

TABLE 21
Hematology Parameters in hTFR^{KI/+} knock in mice

| Com- | | Dose | WBC | RBC | HGB | | | | |
|---|---|---|---|---|---|---|---|---|---|
| pound | | (mg/ | (10\hat{ }3/ | (10\hat{ }12/ | (g/ | HCT | MCV | MCH | MCHC |
| No. | Conjugate | kg) | μL) | L) | dL) | (%) | (fL) | (pg) | (g/dL) |
| PBS | None | 0 | 4‡ | 10‡ | 13‡ | 43‡ | 45‡ | 14‡ | 31‡ |
| 486178 | None | 35 | 4 | 10 | 14 | 45 | 44 | 13 | 30 |
| 1468770 | OKT9 | 3.5 | 5 | 10 | 14 | 46 | 44 | 13 | 31 |
| 1590463 | BCY17868 | 3.5 | 4 | 10 | 14 | 46 | 46 | 14 | 30 |
| 1590463 | BCY17869 | 3.5 | 6 | 10 | 14 | 47 | 45 | 14 | 31 |
| 1590463 | BCY17870 | 3.5 | 5 | 10 | 14 | 46 | 45 | 14 | 30 |
| 1590463 | BCY17871 | 3.5 | 5 | 10 | 14 | 46 | 44 | 13 | 30 |
| 1590463 | BCY17872 | 3.5 | 6 | 10 | 14 | 46 | 44 | 14 | 31 |
| 1590463 | BCY17873 | 3.5 | 6 | 11 | 14 | 47 | 44 | 13 | 30 |
| 1590463 | BCY17874 | 3.5 | 7 | 11 | 14 | 47 | 44 | 13 | 31 |
| 1590463 | BCY17875 | 3.5 | 7 | 11 | 15 | 48 | 45 | 13 | 30 |
| 1590463 | BCY17876 | 3.5 | 6 | 10 | 14 | 47 | 45 | 14 | 30 |
| 1590463 | BCY17877 | 3.5 | 8 | 11 | 14 | 47 | 44 | 13 | 30 |
| 1590463 | BCY17878 | 3.5 | 6 | 11 | 14 | 47 | 44 | 13 | 31 |
| 1590463 | BCY17879 | 3.5 | 5 | 11 | 14 | 48 | 44 | 13 | 30 |
| 1590463 | BCY17880 | 3.5 | 7 | 11 | 15 | 48 | 44 | 13 | 30 |

‡indicates that there were fewer than 4 samples available

TABLE 22
Blood Cell Counts in hTFR^{KI/+} knock in mice

| Com- | | Dose | | | | | | PLT | RETI |
|---|---|---|---|---|---|---|---|---|---|
| pound | | (mg/ | NEU | LYM | MON | EOS | BAS | (10\hat{ }9/ | (10\hat{ }3/ |
| No. | Conjugate | kg) | (%) | (%) | (%) | (%) | (%) | L) | μL) |
| PBS | None | 0 | 11‡ | 82‡ | 6‡ | 0.87‡ | 0.00‡ | 1083‡ | 784‡ |
| 486178 | None | 35 | 9 | 83 | 6 | 1.87 | 0.13 | 1010 | 619 |
| 1468770 | OKT9 | 3.5 | 10 | 85 | 4 | 1.00 | 0.10 | 948 | 620 |
| 1590463 | BCY17868 | 3.5 | 12 | 80 | 6 | 2.33 | 0.00 | 912 | 755 |
| 1590463 | BCY17869 | 3.5 | 7 | 86 | 4 | 2.00 | 0.17 | 1019 | 842 |
| 1590463 | BCY17870 | 3.5 | 8 | 84 | 6 | 2.13 | 0.03 | 1113 | 779 |
| 1590463 | BCY17871 | 3.5 | 14 | 80 | 5 | 1.23 | 0.20 | 1087 | 730 |
| 1590463 | BCY17872 | 3.5 | 10 | 86 | 4 | 0.30 | 0.07 | 991 | 683 |
| 1590463 | BCY17873 | 3.5 | 10 | 81 | 5 | 3.20 | 0.30 | 958 | 727 |
| 1590463 | BCY17874 | 3.5 | 8 | 86 | 4 | 2.07 | 0.13 | 1054 | 715 |
| 1590463 | BCY17875 | 3.5 | 8 | 85 | 4 | 2.53 | 0.20 | 964 | 826 |
| 1590463 | BCY17876 | 3.5 | 9 | 85 | 4 | 2.07 | 0.27 | 974 | 775 |
| 1590463 | BCY17877 | 3.5 | 10 | 82 | 5 | 2.67 | 0.13 | 1018 | 703 |
| 1590463 | BCY17878 | 3.5 | 8 | 84 | 4 | 3.77 | 0.27 | 896 | 826 |
| 1590463 | BCY17879 | 3.5 | 13 | 82 | 4 | 1.33 | 0.07 | 861 | 749 |
| 1590463 | BCY17880 | 3.5 | 10 | 83 | 4 | 3.40 | 0.33 | 966 | 838 |

‡indicates that there were fewer than 4 samples available

### Body and Organ Weights

Body weights of hTFR^{KI/+} mice were measured on days 1 and 22, and the average body weight for each group is presented in the table below. Liver, kidney, and spleen weights were measured on the day the mice were sacrificed (day 22), and the average organ weights for each group are presented in the tables below. Oligomeric compounds that caused any changes in organ weights outside the expected range for modified oligonucleotides were excluded from further studies.

TABLE 23
Body and organ weights (in grams)

| | | | Body | | | | |
|---|---|---|---|---|---|---|---|
| Com- | | Dose | weight (g) | | | | |
| pound | | (mg/ | Day | Day | Organ weight (g) | | |
| No. | Conjugate | kg) | 1 | 22 | Liver | Kidney | Spleen |
| PBS | None | 0 | 23 | 24 | 1.15‡ | 0.28 | 0.09 |
| 486178 | None | 35 | 22 | 24 | 1.23 | 0.28 | 0.09 |
| 1468770 | OKT9 | 3.5 | 27 | 28 | 1.37 | 0.32 | 0.09 |
| 1590463 | BCY17868 | 3.5 | 22 | 22 | 1.14 | 0.28 | 0.09 |
| 1590463 | BCY17869 | 3.5 | 24 | 24 | 1.14 | 0.28 | 0.09 |
| 1590463 | BCY17870 | 3.5 | 22 | 23 | 1.18 | 0.28 | 0.09 |
| 1590463 | BCY17871 | 3.5 | 24 | 24 | 1.17 | 0.30 | 0.08 |
| 1590463 | BCY17872 | 3.5 | 23 | 24 | 1.19 | 0.30 | 0.08 |
| 1590463 | BCY17873 | 3.5 | 23 | 23 | 1.10 | 0.28 | 0.08 |
| 1590463 | BCY17874 | 3.5 | 22 | 22 | 1.03 | 0.27 | 0.09 |
| 1590463 | BCY17875 | 3.5 | 22 | 23 | 1.03 | 0.28 | 0.08 |
| 1590463 | BCY17876 | 3.5 | 22 | 23 | 1.04 | 0.27 | 0.09 |
| 1590463 | BCY17877 | 3.5 | 23 | 23 | 1.04 | 0.29 | 0.16 |
| 1590463 | BCY17878 | 3.5 | 24 | 25 | 1.13 | 0.28 | 0.09 |
| 1590463 | BCY17879 | 3.5 | 23 | 25 | 1.12 | 0.29 | 0.09 |
| 1590463 | BCY17880 | 3.5 | 23 | 24 | 1.09 | 0.29 | 0.08 |

‡indicates that there were fewer than 4 samples available

### Example 7: Activity and Tolerability of Modified Oligonucleotides Conjugated to CD71 Bicycle Ligands, and a Duplexed Modified Oligonucleotide Conjugated to a CD71 Bicycle Ligand in hTFR^{KI/+} Knock in Mice

Activity and tolerability of modified oligonucleotides conjugated to CD71 bicycle ligands was tested in in hTFR^{KI/+} knock in mice (described herein above). In addition, activity of Compound No. 1468770 (described herein above) conjugated to the Fab′ fragments of OKT9 antibody (BioXCell, catalog number: BE0023) that targets human CD71 was tested.

### Treatment

hTFR^{KI/+} mice were divided into groups of 3 mice each. Each mouse received an intravenous administration of conjugated modified oligonucleotide for a total of 3 doses (on Days 1, 8, and 15) at doses indicated in the table below. A group of 3 mice received an intravenous administration of unconjugated modified oligonucleotide, Compound No. 486178, for a total of 3 doses (on Days 1, 8, and 15) at doses indicated in the table below. A group of 4 mice received PBS as a negative control.

### RNA Analysis

The mice were sacrificed four days post final administration (on day 19), and RNA was extracted from various muscle tissues (including quadriceps (Quad), tibialis anterior (TA), diaphragm (Diaphr), triceps, heart, gastrocnemius (gastroc)), aorta, sciatic nerve, and liver tissue for quantitative real time RTPCR analysis to measure amount of mouse DMPK RNA using mouse primer probe set RTS3181 (described herein above). Results are presented as percent mouse DMPK RNA relative to PBS control, normalized to mouse GAPDH (% control).

TABLE 24
Reduction of mouse DMPK RNA in hTFR^{KI/+} knock in mice with modified
oligonucleotides conjugated to CD71 bicycle ligands

| Com- | | Dose | | | | | | | |
|---|---|---|---|---|---|---|---|---|---|
| pound | Con- | (mg/ | mouse DMPK RNA (% control) | | | | | | |
| No. | jugate | kg) | Quad | Gastroc | TA | Triceps | Diaphr | Heart | Liver |
| 486178 | None | 35 | 23 | 29 | 29 | 28 | 20 | 54 | 12 |
| 1468770 | OKT9 | 3.5 | 34 | 35 | 32 | 44 | 21 | 39 | 15 |
| 1590463 | BCY17882 | 3.5 | 49 | 49 | 61 | 56 | 49 | 75 | 43 |
| 1590463 | BCY17890 | 3.5 | 47 | 47 | 67 | 49 | 49 | 81 | 61 |
| 1590463 | BCY17892 | 3.5 | 45 | 46 | 49 | 46 | 33 | 61 | 30 |
| 1590463 | BCY17894 | 3.5 | 44 | 48 | 46 | 48 | 28 | 59 | 25 |
| 1590463 | BCY17896 | 3.5 | 36 | 43 | 37 | 41 | 26 | 62 | 24 |
| 1590463 | BCY17899 | 3.5 | 53 | 59 | 58 | 61 | 40 | 70 | 24 |
| 1590463 | BCY17901 | 3.5 | 26‡ | 32‡ | 24‡ | 27‡ | 19‡ | 43‡ | 28‡ |
| 1614439 | BCY17906 | 3.5 | 50 | 57 | 57 | 55 | 37 | 60 | 30 |
| 1590463 | BCY17904 | 3.5 | 58 | 64 | 61 | 58 | 44 | 75 | 27 |
| 1590463 | BCY17873 | 3.5 | 46 | 53 | 48 | 48 | 32 | 59 | 31 |
| 1602000 | BCY17873 | 3.5 | 50 | 54 | 58 | 52 | 33 | 69 | 28 |
| 486178: | BCY17873 | 3.5 | 60 | 61 | 66 | 56 | 48 | 69 | 40 |
| 1614438 | | | | | | | | | |
| 1609732 | BCY17873 | 3.5 | 45 | 49 | 51 | 46 | 34 | 59 | 25 |

‡indicates fewer than 3 samples available

### Plasma Chemistry Markers

To evaluate the effect of modified oligonucleotides on liver and kidney function, plasma levels of albumin (ALB), alanine aminotransferase (ALT), aspartate aminotransferase (AST), blood urea nitrogen (BUN), total bilirubin (TBIL), total Protein (PROT), Creatine (CREAT), and Creatine Kinase (CK) were measured on the day the mice were sacrificed (day 19) using an automated clinical chemistry analyzer (Hitachi Olympus AU400c, Melville, NY). The results were averaged for each group of mice and are presented in the tables below. Oligomeric compounds that caused changes in the levels of any of the liver or kidney function markers outside the expected range for modified oligonucleotides were excluded from further studies.

TABLE 25
Plasma chemistry markers in hTFR^{KI/+} knock in mice

| | | | Plasma clinical chemistry | | | | | | |
|---|---|---|---|---|---|---|---|---|---|
| Com- | | Dose | | | | TBIL | BUN | PROT | CREAT |
| pound | | (mg/ | ALB | ALT | AST | (mg/ | (mg/ | (g/ | (mg/ |
| No. | Conjugate | kg) | (g/dL) | (U/L) | (U/L) | dL) | dL) | dL) | dL) |
| PBS | None | 0 | 3.32 | 93 | 220 | 0.20 | 24 | 5.25 | 0.12 |
| 486178 | None | 35 | 3.53 | 40 | 106 | 0.16 | 25 | 5.67 | 0.15 |
| 1468770 | OKT9 | 3.5 | 3.56 | 34 | 74 | 0.15 | 26 | 5.63 | 0.15 |
| 1590463 | BCY17882 | 3.5 | 3.67 | 76 | 149 | 0.15 | 20 | 5.53 | 0.15 |
| 1590463 | BCY17890 | 3.5 | 3.76 | 36 | 127 | 0.15 | 24 | 5.73 | 0.16 |
| 1590463 | BCY17892 | 3.5 | 3.10 | 35 | 103 | 0.13 | 21 | 4.80 | 0.11 |
| 1590463 | BCY17894 | 3.5 | 3.13 | 102 | 257 | 0.19 | 22 | 5.00 | 0.14 |
| 1590463 | BCY17896 | 3.5 | 3.91 | 72 | 133 | 0.16 | 27 | 6.40 | 0.23 |
| 1590463 | BCY17899 | 3.5 | 3.61 | 54 | 134 | 0.15 | 24 | 5.83 | 0.17 |
| 1590463 | BCY17901 | 3.5 | 2.85‡ | 56‡ | 180‡ | 0.11‡ | 19‡ | 4.70‡ | 0.12‡ |
| 1614439 | BCY17906 | 3.5 | 3.13 | 53 | 153 | 0.14 | 21 | 4.93 | 0.12 |
| 1590463 | BCY17904 | 3.5 | 3.65 | 52 | 103 | 0.12 | 23 | 6.03 | 0.18 |
| 1590463 | BCY17873 | 3.5 | 3.17 | 40 | 134 | 0.16 | 22 | 5.17 | 0.13 |
| 1602000 | BCY17873 | 3.5 | 3.54 | 33 | 78 | 0.13 | 23 | 5.87 | 0.18 |
| 486178: | BCY17873 | 3.5 | 3.25 | 32 | 119 | 0.14 | 20 | 5.17 | 0.13 |
| 1614438 | | | | | | | | | |
| 1609732 | BCY17873 | 3.5 | 3.42 | 58 | 150 | 0.14 | 22 | 5.50 | 0.17 |

‡indicates fewer than 3 samples available

### Hematology Assays

Blood obtained from mice on the day the mice were sacrificed (day 19) were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), reticulocytes, and platelets were evaluated. The results are presented in the tables below. Oligomeric compounds that caused changes in the blood cell count outside the expected range were excluded in further studies.

TABLE 26
Hematology Parameters in hTFR^{KI/+} knock in mice

| Com- | | Dose | WBC | RBC | HGB | | | | |
|---|---|---|---|---|---|---|---|---|---|
| pound | | (mg/ | (10\hat{ }3/ | (10\hat{ }12/ | (g/ | HCT | MCV | MCH | MCHC |
| No. | Conjugate | kg) | μL) | L) | dL) | (%) | (fL) | (pg) | (g/dL) |
| PBS | None | 0 | 7 | 12 | 15 | 46 | 39 | 12 | 32 |
| 486178 | None | 35 | 7 | 12 | 14 | 45 | 40 | 13 | 32 |
| 1468770 | OKT9 | 3.5 | 9 | 12 | 15 | 47 | 40 | 12 | 31 |
| 1590463 | BCY17882 | 3.5 | 5 | 11 | 14 | 45 | 41 | 13 | 31 |
| 1590463 | BCY17890 | 3.5 | 4 | 11 | 14 | 44 | 40 | 13 | 32 |
| 1590463 | BCY17892 | 3.5 | 8 | 12 | 15 | 46 | 40 | 13 | 32 |
| 1590463 | BCY17894 | 3.5 | 12 | 12 | 14 | 46 | 40 | 13 | 32 |
| 1590463 | BCY17896 | 3.5 | 8 | 12 | 15 | 46 | 39 | 12 | 32 |
| 1590463 | BCY17899 | 3.5 | 7 | 12 | 15 | 46 | 39 | 12 | 32 |
| 1590463 | BCY17901 | 3.5 | 12‡ | 11‡ | 14‡ | 47‡ | 42‡ | 13‡ | 30‡ |
| 1614439 | BCY17906 | 3.5 | 8 | 12 | 15 | 47 | 40 | 13 | 32 |
| 1590463 | BCY17904 | 3.5 | 9 | 11 | 14 | 46 | 40 | 13 | 31 |
| 1590463 | BCY17873 | 3.5 | 9 | 12 | 15 | 47 | 40 | 12 | 31 |
| 1602000 | BCY17873 | 3.5 | 10 | 12 | 14 | 47 | 41 | 13 | 31 |
| 486178: | BCY17873 | 3.5 | 10 | 12 | 15 | 48 | 40 | 12 | 31 |
| 1614438 | | | | | | | | | |
| 1609732 | BCY17873 | 3.5 | 11‡ | 12‡ | 15‡ | 47‡ | 40‡ | 12‡ | 31‡ |

‡indicates that there are fewer than 3 samples available

TABLE 27
Blood Cell Counts in hTFR^{KI/+} knock in mice

| Com- | | Dose | | | | | | PLT | RETI |
|---|---|---|---|---|---|---|---|---|---|
| pound | Con- | (mg/ | NEU | LYM | MON | EOS | BAS | (10\hat{ }9/ | (10\hat{ }3/ |
| No. | jugate | kg) | (%) | (%) | (%) | (%) | (%) | L) | μL) |
| PBS | None | 0 | 11 | 83 | 5 | 1.28 | 0.15 | 1095 | 437 |
| 486178 | None | 35 | 12 | 82 | 4 | 1.37 | 0.40 | 1335 | 462 |
| 1468770 | OKT9 | 3.5 | 10 | 85 | 4 | 0.87 | 0.30 | 1169 | 430 |
| 1590463 | BCY17882 | 3.5 | 10 | 85 | 3 | 1.03 | 0.23 | 930 | 382 |
| 1590463 | BCY17890 | 3.5 | 13 | 82 | 4 | 1.40 | 0.33 | 942 | 354 |
| 1590463 | BCY17892 | 3.5 | 10 | 85 | 3 | 1.50 | 0.20 | 1119 | 401 |
| 1590463 | BCY17894 | 3.5 | 30 | 61 | 9 | 0.33 | 0.03 | 1081 | 416 |
| 1590463 | BCY17896 | 3.5 | 15 | 79 | 6 | 0.33 | 0.00 | 1246 | 441 |
| 1590463 | BCY17899 | 3.5 | 9 | 88 | 2 | 1.20 | 0.10 | 1185 | 428 |
| 1590463 | BCY17901 | 3.5 | 24‡ | 64‡ | 10‡ | 1.40‡ | 0.10‡ | 1122‡ | 411‡ |
| 1614439 | BCY17906 | 3.5 | 11 | 84 | 3 | 1.87 | 0.23 | 1127 | 458 |
| 1590463 | BCY17904 | 3.5 | 16 | 75 | 7 | 1.20 | 0.13 | 1238 | 417 |
| 1590463 | BCY17873 | 3.5 | 9 | 86 | 4 | 1.30 | 0.13 | 1444 | 501 |
| 1602000 | BCY17873 | 3.5 | 10 | 84 | 4 | 2.30 | 0.13 | 1165 | 414 |
| 486178: | BCY17873 | 3.5 | 10 | 85 | 3 | 1.57 | 0.13 | 1174 | 450 |
| 1614438 | | | | | | | | | |
| 1609732 | BCY17873 | 3.5 | 10‡ | 85‡ | 3‡ | 1.90‡ | 0.20‡ | 1203‡ | 442‡ |

‡indicates that there are fewer than 3 samples available

### Body and Organ Weights

Body weights of hTFR^{KI/+} mice were measured on days 1 and 19, and the average body weight for each group is presented in the table below. Liver, kidney, and spleen weights were measured on the day the mice were sacrificed (day 19), and the average organ weights for each group are presented in the tables below. Oligomeric compounds that caused any changes in organ weights outside the expected range for modified oligonucleotides were excluded from further studies.

TABLE 28
Body and organ weights (in grams)

| | | | Body | | | | |
|---|---|---|---|---|---|---|---|
| Com- | | Dose | weight (g) | | | | |
| pound | Con- | (mg/ | Day | Day | Organ weight (g) | | |
| No. | jugate | kg) | 1 | 19 | Liver | Kidney | Spleen |
| PBS | None | 0 | 29 | 30 | 1.40 | 0.40 | 0.07 |
| 486178 | None | 35 | 28 | 29 | 1.45 | 0.37 | 0.08 |
| 1468770 | OKT9 | 3.5 | 31 | 33 | 1.59 | 0.43 | 0.08 |
| 1590463 | BCY17882 | 3.5 | 22 | 23 | 1.11 | 0.31 | 0.09 |
| 1590463 | BCY17890 | 3.5 | 22 | 24 | 1.16 | 0.30 | 0.08 |
| 1590463 | BCY17892 | 3.5 | 27 | 29 | 1.48 | 0.37 | 0.08 |
| 1590463 | BCY17894 | 3.5 | 29 | 30 | 1.49 | 0.40 | 0.08 |
| 1590463 | BCY17896 | 3.5 | 29 | 30 | 1.53 | 0.43 | 0.08 |
| 1590463 | BCY17899 | 3.5 | 29 | 30 | 1.51 | 0.43 | 0.08 |
| 1590463 | BCY17901 | 3.5 | 30‡ | 32‡ | 1.69‡ | 0.44‡ | 0.08‡ |
| 1614439 | BCY17906 | 3.5 | 26 | 27 | 1.36 | 0.40 | 0.07 |
| 1590463 | BCY17904 | 3.5 | 28 | 29 | 1.52 | 0.41 | 0.08 |
| 1590463 | BCY17873 | 3.5 | 29 | 30 | 1.47 | 0.42 | 0.07 |
| 1602000 | BCY17873 | 3.5 | 33 | 35 | 1.80 | 0.44 | 0.09 |
| 486178: | BCY17873 | 3.5 | 29 | 31 | 1.43 | 0.41 | 0.08 |
| 1614438 | | | | | | | |
| 1609732 | BCY17873 | 3.5 | 30 | 32 | 1.52 | 0.42 | 0.08 |

‡indicates that there are fewer than 3 samples available

### Example 8: Activity and Tolerability of Modified Oligonucleotides Conjugated to CD71 Bicycle Ligands in hTFR^{KI/+} Knock in Mice, Multiple Dose

The activity and tolerability of modified oligonucleotides conjugated to CD71 bicycle ligands were tested in in hTFR^{KI/+} knock in mice (described herein above). In addition, activity and tolerability of Compound No. 1468770 (described herein above) conjugated to the Fab′ fragments of OKT9 antibody (BioXCell, catalog number: BE0023) that targets human CD71 were tested.

### Treatment

hTFR^{KI/+} mice were divided into groups of 3-4 mice each. Each mouse received an intravenous (i.v.) administration or subcutaneous (s.c.) administration of conjugated modified oligonucleotide for a total of 3 doses (on Days 1, 5, and 9) at doses indicated in the table below. A group of 3 mice received an intravenous administration of unconjugated modified oligonucleotide, Compound No. 486178, for a total of 3 doses (on Days 1, 5, and 9) at doses indicated in the table below. A group of 4 mice received PBS as a negative control.

### RNA Analysis

The mice were sacrificed six days post final administration (on day 15), and RNA was extracted from various muscle tissues (including quadriceps (Quad), tibialis anterior (TA), diaphragm, triceps, heart, gastrocnemius (gastroc)), aorta, sciatic nerve, and liver tissue for quantitative real time RTPCR analysis to measure amount of mouse DMPK RNA using mouse primer probe set RTS3181 (described herein above). Results are presented as percent mouse DMPK RNA relative to PBS control, normalized to mouse GAPDH (% control).

TABLE 29
Reduction of mouse DMPK RNA in hTFR^{KI/+} knock in mice with
modified oligonucleotides conjugated to CD71 bicycle ligands

| | | | | Quad- | Gastroc- | Tibialis | Dia- | |
|---|---|---|---|---|---|---|---|---|
| | | | | riceps | nemius | Anterior | phragm | Heart |
| | | | | mouse | mouse | mouse | mouse | mouse |
| | | Mode | | DMPK | DMPK | DMPK | DMPK | DMPK |
| Com- | | of | | RNA | RNA | RNA | RNA | RNA |
| pound | | Admin- | Dose | (% | (% | (% | (% | (% |
| No. | Conjugate | istration | (mg/kg) | control) | control) | control) | control) | control) |
| 486178 | None | i.v. | 5 | 79 | 82 | 77 | 61 | 88 |
| | | | 20 | 35 | 40 | 42 | 28 | 63 |
| | | | 50 | 20 | 20 | 23 | 16 | 48 |
| 1468770 | OKT9 | i.v | 1 | 84 | 91 | 87 | 67 | 73 |
| | | | 3.5 | 26 | 30 | 27 | 21 | 37 |
| | | | 10 | 17 | 17 | 18 | 11 | 34 |
| 1590463 | BCY17901 | i.v. | 1 | 79 | 74 | 71 | 57 | 75 |
| | | | 3.5 | 30 | 28 | 29 | 21 | 48 |
| | | | 10 | 14 | 15 | 14 | 14 | 44 |
| 1590463 | BCY17896 | i.v. | 1 | 74 | 75 | 84 | 60 | 81 |
| | | | 3.5 | 40 | 40 | 42 | 25 | 61 |
| | | | 10 | 23 | 28 | 28 | 16 | 56 |
| 1590463 | BCY17896 | s.c. | 3.5 | 63 | 61 | 59 | 48 | 76 |

TABLE 30
Reduction of mouse DMPK RNA in hTFR^{KI/+} knock in mice with
modified oligonucleotides conjugated to CD71 bicycle ligands

| | | Mode | | Gas- | Tibialis | | |
|---|---|---|---|---|---|---|---|
| | | of | Quad- | troc- | An- | Dia- | |
| | | Ad- | riceps | nemius | terior | phragm | Heart |
| Com- | | min- | ED50 | ED50 | ED50 | ED50 | ED50 |
| pound | | istra- | (mg/ | (mg/ | (mg/ | (mg/ | (mg/ |
| No. | Conjugate | tion | kg) | kg) | kg) | kg) | kg) |
| 486178 | None | i.v. | 13.5 | 15.4 | 15.2 | 8.1 | 39.5 |
| 1468770 | OKT9 | i.v | 2.2 | 2.5 | 2.4 | 1.6 | 2.8 |
| 1590463 | BCY17901 | i.v. | 2.2 | 2 | 1.9 | 1.2 | N.C |
| 1590463 | BCY17896 | i.v. | 2.6 | 2.9 | 3.3 | 1.4 | N.C |

### Plasma Chemistry Markers

To evaluate the effect of modified oligonucleotides on liver and kidney function, plasma levels of albumin (ALB), alanine aminotransferase (ALT), aspartate aminotransferase (AST), blood urea nitrogen (BUN), total bilirubin (TBIL), total Protein (PROT), Creatine (CREAT), and Creatine Kinase (CK) were measured on the day the mice were sacrificed (day 15) using an automated clinical chemistry analyzer (Hitachi Olympus AU400c, Melville, NY). The results were averaged for each group of mice and are presented in the tables below. Oligomeric compounds that caused changes in the levels of any of the liver or kidney function markers outside the expected range for modified oligonucleotides were excluded from further studies.

TABLE 31
Plasma chemistry markers in hTFR^{KI/+} knock in mice

| | | Mode | | | | | | | | |
|---|---|---|---|---|---|---|---|---|---|---|
| Com- | | of | Dose | Plasma clinical chemistry | | | | | | |
| pound | Con- | Admin- | (mg/ | ALB | ALT | AST | TBIL | BUN | PROT | CREAT |
| No. | jugate | istration | kg) | (g/dL) | (U/L) | (U/L) | (mg/dL) | (mg/dL) | (g/dL) | (mg/dL) |
| PBS | None | i.v. | 0 | 2.86 | 49 | 121 | 0.17 | 27 | 4.90 | 0.15 |
| 486178 | None | i.v. | 5 | 2.78 | 31 | 83 | 0.12 | 27 | 4.80 | 0.15 |
| | | | 20 | 2.78 | 44 | 114 | 0.13 | 28 | 4.83 | 0.16 |
| | | | 50 | 2.78 | 46 | 115 | 0.14 | 26 | 4.83 | 0.14 |
| 1468770 | OKT9 | i.v. | 1 | 2.85 | 32 | 144 | 0.16 | 25 | 4.90 | 0.15 |
| | | | 3.5 | 2.73 | 34 | 56 | 0.15 | 24 | 4.70 | 0.15 |
| | | | 10 | 2.67 | 43 | 109 | 0.15 | 26 | 4.67 | 0.16 |
| 1590463 | BCY17901 | i.v. | 1 | 2.76 | 65 | 107 | 0.13 | 23 | 4.73 | 0.15 |
| | | | 3.5 | 2.86 | 59 | 206 | 0.13 | 23 | 4.97 | 0.18 |
| | | | 10 | 2.63 | 35 | 98 | 0.14 | 23 | 4.53 | 0.15 |
| 1590463 | BCY17896 | i.v. | 1 | 2.82 | 40 | 111 | 0.12 | 24 | 4.83 | 0.16 |
| | | | 3.5 | 2.77 | 44 | 161 | 0.10 | 22 | 4.70 | 0.16 |
| | | | 10 | 2.70 | 34 | 119 | 0.11 | 22 | 4.57 | 0.16 |
| 1590463 | BCY17896 | s.c. | 3.5 | 2.65 | 71 | 129 | 0.11 | 20 | 4.63 | 0.14 |

### Hematology Assays

Blood obtained from mice on the day the mice were sacrificed (day 15) were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscularhemoglobin concentration (MCHC). Individual white blood cell counts, such as that of monocytes (MON), neutrophils (NEU), lymphocytes (LYM), eosinophils (EOS), basophils (BAS), reticulocytes, and platelets were evaluated. The results are presented in the tables below. Oligomeric compounds that caused changes in the blood cell count outside the expected range were excluded in further studies.

TABLE 32
Hematology Parameters in hTFR^{KI/+} knock in mice

| Com- | | Mode of | Dose | WBC | RBC | | | | | |
|---|---|---|---|---|---|---|---|---|---|---|
| pound | | Admin- | (mg/ | (10\hat{ }3/ | (10\hat{ }12/ | HGB | HCT | MCV | MCH | MCHC |
| No. | Conjugate | istration | kg) | μL) | L) | (g/dL) | (%) | (fL) | (pg) | (g/dL) |
| PBS | None | i.v. | 0 | 6 | 11 | 14 | 45 | 40 | 13 | 32 |
| 486178 | None | i.v. | 5 | 8 | 12 | 15 | 47 | 41 | 13 | 31 |
| | | | 20 | 7 | 11 | 15 | 46 | 40 | 13 | 32 |
| | | | 50 | 7 | 11 | 15 | 47 | 41 | 13 | 32 |
| 1468770 | OKT9 | i.v. | 1 | 6 | 12 | 15 | 47 | 40 | 13 | 32 |
| | | | 3.5 | 7 | 12 | 15 | 46 | 39 | 12 | 32 |
| | | | 10 | 11 | 12 | 15 | 47 | 40 | 13 | 31 |
| 1590463 | BCY17901 | i.v. | 1 | 7 | 12 | 15 | 48 | 40 | 13 | 31 |
| | | | 3.5 | 7 | 12 | 15 | 48 | 40 | 13 | 32 |
| | | | 10 | 9 | 11 | 15 | 47 | 41 | 13 | 31 |
| 1590463 | BCY17896 | i.v. | 1 | 8 | 12 | 15 | 47 | 40 | 13 | 32 |
| | | | 3.5 | 7 | 12 | 15 | 49 | 41 | 13 | 31 |
| | | | 10 | 9 | 11 | 15 | 47 | 41 | 13 | 31 |
| 1590463 | BCY17896 | s.c. | 3.5 | 10 | 11 | 14 | 47 | 42 | 13 | 31 |

TABLE 33
Blood Cell Counts in hTFR^{KI/+} knock in mice

| Com- | | Mode of | Dose | | | | | | PLT | RETI |
|---|---|---|---|---|---|---|---|---|---|---|
| pound | | Admin- | (mg/ | NEU | LYM | MON | EOS | BAS | (10\hat{ }9/ | (10\hat{ }3/ |
| No. | Conjugate | istration | kg) | (%) | (%) | (%) | (%) | (%) | L) | μL) |
| PBS | None | i.v. | 0 | 9 | 83 | 7 | 2 | 0.03 | 1220 | 422 |
| 486178 | None | i.v. | 5 | 8 | 84 | 6 | 1 | 0.00 | 1240 | 416 |
| | | | 20 | 10 | 80 | 6 | 3 | 0.10 | 1004 | 436 |
| | | | 50 | 12 | 78 | 9 | 1 | 0.03 | 1260 | 417 |
| 1468770 | OKT9 | i.v. | 1 | 9 | 83 | 6 | 2 | 0.20 | 1150 | 440 |
| | | | 3.5 | 8 | 84 | 6 | 2 | 0.13 | 1200 | 452 |
| | | | 10 | 7 | 84 | 6 | 3 | 0.03 | 1117 | 477 |
| 1590463 | BCY17901 | i.v. | 1 | 7 | 86 | 5 | 2 | 0.10 | 1263 | 448 |
| | | | 3.5 | 7 | 86 | 6 | 2 | 0.07 | 1149 | 451 |
| | | | 10 | 10 | 82 | 6 | 2 | 0.03 | 1112 | 417 |
| 1590463 | BCY17896 | i.v. | 1 | 8 | 85 | 5 | 2 | 0.07 | 1170 | 439 |
| | | | 3.5 | 13 | 79 | 5 | 4 | 0.00 | 1073 | 452 |
| | | | 10 | 8 | 84 | 6 | 2 | 0.07 | 1087 | 389 |
| 1590463 | BCY17896 | s.c. | 3.5 | 11 | 79 | 7 | 2 | 0.13 | 1203 | 446 |

### Body and Organ Weights

Body weights of hTFR^{KI/+} mice were measured on days 1 and 15, and the average body weight for each group is presented in the table below. Liver, kidney, and spleen weights were measured on the day the mice were sacrificed (day 15), and the average organ weights for each group are presented in the tables below. Oligomeric compounds that caused any changes in organ weights outside the expected range for modified oligonucleotides were excluded from further studies.

TABLE 34
Body and organ weights (in grams)

| | | Mode | | | | | | |
|---|---|---|---|---|---|---|---|---|
| | | of | | Body | | | | |
| | | Ad- | | weight | | | | |
| Com- | | min- | Dose | (g) | | Organ weight (g) | | |
| pound | | istra- | (mg/ | Day | Day | | Kid- | |
| No. | Conjugate | tion | kg) | 1 | 15 | Liver | ney | Spleen |
| PBS | | i.v. | 0 | 29 | 30 | 1.64 | 0.38 | 0.07 |
| 486178 | None | i.v. | 5 | 27 | 28 | 1.52 | 0.37 | 0.07 |
| | | | 20 | 28 | 30 | 1.71 | 0.36 | 0.08 |
| | | | 50 | 25 | 28 | 1.53 | 0.33 | 0.07 |
| 1468770 | OKT9 | i.v. | 1 | 25 | 26 | 1.40 | 0.33 | 0.06 |
| | | | 3.5 | 28 | 29 | 1.58 | 0.38 | 0.07 |
| | | | 10 | 28 | 29 | 1.61 | 0.35 | 0.07 |
| 1590463 | BCY17901 | i.v. | 1 | 26 | 27 | 1.49 | 0.36 | 0.07 |
| | | | 3.5 | 36 | 37 | 1.89 | 0.41 | 0.08 |
| | | | 10 | 23 | 25 | 1.35 | 0.35 | 0.07 |
| 1590463 | BCY17896 | i.v. | 1 | 27 | 28 | 1.49 | 0.35 | 0.07 |
| | | | 3.5 | 28 | 29 | 1.53 | 0.37 | 0.07 |
| | | | 10 | 24 | 25 | 1.39 | 0.33 | 0.07 |
| 1590463 | BCY17896 | s.c. | 3.5 | 26 | 27 | 1.50 | 0.35 | 0.08 |

### Example 9: Activity and Tolerability of RNAi Compounds Conjugated to CD71 Bicycle Ligands in hTFR^{KI/+} Knock in Mice

The activity and tolerability of RNAi compounds conjugated to CD71 bicycle ligands were tested in in hTFR^{KI/+} knock in mice (described herein above). In addition, activity and tolerability of Compound No. 1468770 (described herein above) conjugated to the Fab′ fragments of OKT9 antibody (BioXCell, catalog number: BE0023) that targets human CD71 were tested.

### Treatment

hTFR^{KI/+} mice were divided into groups of 3 mice each. Each mouse received an intravenous administration of conjugated RNAi compound for a total of 3 doses (on Days 1, 8, and 15) at doses indicated in the table below. A group of 4 mice received PBS as a negative control.

### RNA Analysis

The mice were sacrificed four days post final administration (on day 19), and RNA was extracted from various muscle tissues (including quadriceps (Quad), tibialis anterior (TA), diaphragm (Dia.), triceps, heart, gastrocnemius (gastroc)), aorta, and liver tissue for quantitative real time RTPCR analysis to measure amount of mouse HPRT RNA using mouse primer probe set RTS43125 (forward sequence CTCCTCAGACCGCTTTTTGC, designated herein as SEQ ID NO: 19; reverse sequence TAACCTGGTTCATCATCGCTAATC, designated herein as SEQ ID NO: 20; probe sequence CCGTCATGCCGACCCGCAGT, designated herein as SEQ ID NO: 21). Results are presented as percent mouse HPRT RNA relative to PBS control, normalized to mouse GAPDH (% control).

TABLE 35
Reduction of mouse HPRT RNA in hTFR^{KI/+} knock in mice with
RNAi compound conjugated to CD71 bicycle ligands

| | | Dose | mouse HPRT RNA (% control) | | | | | | |
|---|---|---|---|---|---|---|---|---|---|
| Compound No. | Conjugate | (mg/kg) | Quad | Gastroc | TA | Triceps | Dia. | Heart | Liver |
| 1453015:1547771 | OKT9 | 3.5 | 23 | 22 | 26 | 24 | 36 | 27 | 13 |
| 1453015:1550983 | BCY17873 | 3.5 | 27 | 24 | 32 | 30 | 48 | 34 | 57 |

### Plasma Chemistry Markers

To evaluate the effect of modified oligonucleotides on liver and kidney function, plasma levels of albumin (ALB), alanine aminotransferase (ALT), aspartate aminotransferase (AST), blood urea nitrogen (BUN), total bilirubin (TBIL), total Protein (PROT), Creatine (CREAT), and Creatine Kinase (CK) were measured on the day the mice were sacrificed (day 19) using an automated clinical chemistry analyzer (Hitachi Olympus AU400c, Melville, NY). The results were averaged for each group of mice and are presented in the tables below. Oligomeric compounds that caused changes in the levels of any of the liver or kidney function markers outside the expected range for modified oligonucleotides were excluded from further studies.

TABLE 36
Plasma chemistry markers in hTFR^{KI/+} knock in mice

| | | | Plasma clinical chemistry | | | | | | |
|---|---|---|---|---|---|---|---|---|---|
| | | Dose | ALB | ALT | AST | TBIL | BUN | PROT | CREAT |
| Compound No. | Conjugate | (mg/kg) | (g/dL) | (U/L) | (U/L) | (mg/dL) | (mg/dL) | (g/dL) | (mg/dL) |
| PBS | None | 0 | 3.32 | 93 | 220 | 0.20 | 24 | 5.25 | 0.12 |
| 1453015:1547771 | OKT9 | 3.5 | 3.17 | 54 | 219 | 0.19 | 21 | 4.80 | 0.11 |
| 1453015:1550983 | BCY17873 | 3.5 | 3.31 | 39 | 143 | 0.14 | 21 | 5.13 | 0.15 |

### Hematology Assays

Blood obtained from mice on the day the mice were sacrificed (day 19) were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), reticulocytes, and platelets were evaluated. The results are presented in the tables below. Oligomeric compounds that caused changes in the blood cell count outside the expected range were excluded in further studies.

TABLE 37
Hematology Parameters in hTFR^{KI/+} knock in mice

| | | Dose | WBC | RBC | HGB | HCT | MCV | MCH | MCHC |
|---|---|---|---|---|---|---|---|---|---|
| Compound No. | Conjugate | (mg/kg) | (10\hat{ }3/μL) | (10\hat{ }12/L) | (g/dL) | (%) | (fL) | (pg) | (g/dL) |
| PBS | None | 0 | 7 | 12 | 15 | 46 | 39 | 12 | 32 |
| 1453015:1547771 | OKT9 | 3.5 | 5 | 11 | 14 | 44 | 40 | 13 | 32 |
| 1453015:1550983 | BCY17873 | 3.5 | 9 | 11 | 14 | 45 | 40 | 13 | 31 |

TABLE 38
Blood Cell Counts in hTFR^{KI/+} knock in mice

| | | Dose | NEU | LYM | MON | EOS | BAS | PLT | RETI |
|---|---|---|---|---|---|---|---|---|---|
| Compound No. | Conjugate | (mg/kg) | (%) | (%) | (%) | (%) | (%) | (10\hat{ }9/L) | (10\hat{ }3/μL) |
| PBS | None | 0 | 11 | 83 | 5 | 1.28 | 0.15 | 1095 | 437 |
| 1453015:1547771 | OKT9 | 3.5 | 12 | 82 | 3 | 1.77 | 0.27 | 1039 | 358 |
| 1453015:1550983 | BCY17873 | 3.5 | 10 | 84 | 4 | 1.67 | 0.10 | 939 | 370 |

### Body and Organ Weights

Body weights of hTFR^{KI/+} mice were measured on days 1 and 19, and the average body weight for each group is presented in the table below. Liver, kidney, and spleen weights were measured on the day the mice were sacrificed (day 19), and the average organ weights for each group are presented in the tables below. Oligomeric compounds that caused any changes in organ weights outside the expected range for modified oligonucleotides were excluded from further studies.

TABLE 39
Body and organ weights (in grams)

| | | Dose | Body weight (g) | | Organ weight (g) | | |
|---|---|---|---|---|---|---|---|
| Compound No. | Conjugate | (mg/kg) | Day 1 | Day 19 | Liver | Kidney | Spleen |
| PBS | None | 0 | 29 | 30 | 1.40 | 0.40 | 0.07 |
| 1453015:1547771 | OKT9 | 3.5 | 21 | 22 | 1.11 | 0.28 | 0.08 |
| 1453015:1550983 | BCY17873 | 3.5 | 23 | 24 | 1.19 | 0.33 | 0.09 |

### Example 10: Design, Activity and Tolerability of Modified Oligonucleotides Conjugated to CD71 Bicycle Ligands in hTFR^{KI/+} Knock in Mice with Various Linker Chemistries

The activity and tolerability of modified oligonucleotides conjugated to CD71 bicycle ligands was tested in in hTFR^{KI/+} knock in mice (described herein above). Several CD71 bicycle ligands were designed with a C-terminal extension to increase the distance between the oligonucleotide and the polypeptide loops of the bicycle ligand, as indicated in the table below.

TABLE 40
Bicycle ligands

| Bicycle | | N-terminal | C-terminal | SEQ ID |
|---|---|---|---|---|
| Ligand ID | Bicycle Notation (N to C) | modification | modification | NO: |
| BCY19405 | CSPDAHLGCISYCEPW[PEG10][K(N₃)] | acetyl | amidated | 52 |
| BCY19406 | CSPDAHLGCISYCEPW[PEG24][K(N₃)] | acetyl | amidated | 52 |
| BCY19407 | CSPDAHLGCISYCEPWGGSGGS[K(N₃)] | acetyl | amidated | 79 |
| BCY19409 | (CSPDAHLGCISYCEPW)₂-[Biv] | acetyl | amidated | 194 |

[PEG10] represents ten repeats of ethylene glycol; [PEG24] represents 24 repeats of ethylene glycol, and Biv represents the bivalent linker according to the following structure, which is attached to two polypeptides:
Bivalent linker

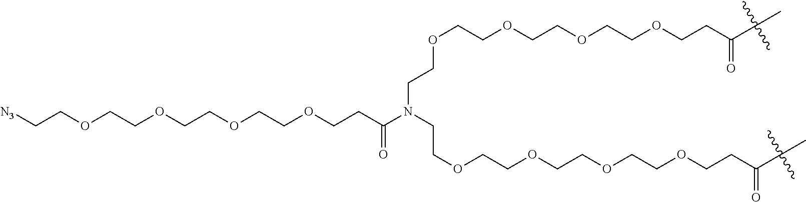 [SMILES: *C(=O)CCOCCOCCOCCOCCN(CCOCCOCCOCCOCCC(*)=O)C(=O)CCOCCOCCOCCOCCN=[N+]=[N-]]

### Treatment

hTFR^{KI/+} mice were divided into groups of 4 mice each. Each mouse received an intravenous administration of conjugated modified oligonucleotide for a total of 3 doses (on Days 1, 8, and 15) at doses indicated in the table below. A group of 4 mice received an intravenous administration of unconjugated modified oligonucleotide, Compound No. 486178, for a total of 3 doses (on Days 1, 8, and 15) at doses indicated in the table below. A group of 4 mice received PBS as a negative control.

### RNA Analysis

The mice were sacrificed 7 days post final administration (on day 22), and RNA was extracted from various muscle tissues (including quadriceps (Quad), tibialis anterior (TA), diaphragm (Diaphr), heart, gastrocnemlus (gastroc)), aorta, sciatic nerve, and liver tissue for quantitative real time RTPCR analysis to measure amount of mouse DMPK RNA using mouse primer probe set RTS3181 (described herein above). Results are presented as percent mouse DMPK RNA relative to PBS control, normalized to mouse GAPDH (% ocontrol).

TABLE 41
Reduction of mouse DMPK RNA in hTFR^{KI/+} knock in mice with modified
oligonucleotides conjugated to CD71 bicycle ligands

| | | | | mouse DMPK RNA (% control) | | | | | | |
|---|---|---|---|---|---|---|---|---|---|---|
| Compound | Bicycle | C-terminal | Dose | | | | Sciatic | | | |
| No. | Ligand | extension | (mg/kg) | Quad | Gastroc | TA | Nerve | Diaphr | Heart | Liver |
| PBS | None | None | 0 | 100 | 100 | 100 | 100 | 100 | 100 | 100 |
| 486178 | None | None | 35 | 26 | 26 | 35 | 47 | 20 | 57 | 13 |
| 1590463 | BCY17873 | None | 3.5 | 44 | 48 | 63 | 77 | 29 | 72 | 32 |
| 1590463 | BCY19405 | PEG10 | 3.5 | 44 | 49 | 49 | 75 | 32 | 75 | 32 |
| 1590463 | BCY19406 | PEG24 | 3.5 | 49 | 48 | 61 | 67 | 32 | 72 | 37 |
| 1590463 | BCY19407 | GGSGGS | 3.5 | 37 | 41 | 50 | 78 | 26 | 69 | 35 |
| | | extension | | | | | | | | |
| 1590463 | BCY19409 | Bivalent | 3.5 | 44 | 46 | 52 | 77 | 30 | 63 | 38 |
| | | linker | | | | | | | | |

### Body Weights

Body weights of hTFR^{KI/+} mice were measured on days 1 and 22, and the average body weight for each group is presented in the table below.

TABLE 42
Body weights (in grams)

| Compound | Bicycle | Peptide | Dose | Body weight (g) | |
|---|---|---|---|---|---|
| No. | Ligand | Linker | (mg/kg) | Day 1 | Day 22 |
| PBS | None | None | 0 | 30 | 30 |
| 486178 | None | None | 35 | 33 | 35 |
| 1590463 | BCY17873 | None | 3.5 | 29 | 30 |
| 1590463 | BCY19405 | PEG10 | 3.5 | 33 | 34 |
| 1590463 | BCY19406 | PEG24 | 3.5 | 34 | 35 |
| 1590463 | BCY19407 | GGSGGS | 3.5 | 31 | 32 |
| | | extension | | | |
| 1590463 | BCY19409 | Bivalent | 3.5 | 28 | 29 |

### Example 11: Activity of Modified Oligonucleotides Conjugated to CD71 Bicycle Ligands in hTFR^{KI/+} Knock in Mice, Single Dose

The activity of modified oligonucleotides conjugated to CD71 bicycle ligands was tested in heterozygous hTFR^{KI/+} knock in mice (described herein above).

### Treatment

hTFR^{KI/+} mice were divided into groups of 4 mice each. Each mouse received an intravenous (i.v.) administration of 3.5 mg/kg of conjugated modified oligonucleotide for a total of 3 doses (on Days 1, 8, and 15). A group of 4 mice received an intravenous administration of 35 mg/kg of unconjugated modified oligonucleotide, Compound No. 486178, for a total of 3 doses (on Days 1, 8, and 15). A group of 4 mice received PBS as a negative control.

### RNA Analysis

The mice were sacrificed one week post final administration (on day 22), and RNA was extracted from various muscle tissues, including quadriceps (quad), tibialis anterior (TA), gastrocnemius (gastroc), heart, and diaphragm, liver tissue, and sciatic nerve for quantitative real time RTPCR analysis to measure amount of mouse DMPK RNA using mouse primer probe set RTS3181 (described herein above). Mouse DMPK RNA levels were normalized to mouse GAPDH. Mouse GAPDH was amplified using mouse primer probe set mGapdh_LTS00102 (described herein above). Results are presented as percent mouse DMPK RNA relative to the amount of mouse DMPK RNA in PBS treated control animals, normalized to mouse GAPDH RNA (% control).

TABLE 43
Reduction of mouse DMPK RNA in hTFR^{KI/+} knock-in mice with modified
oligonucleotides conjugated to CD71 bicycle ligands

| Com- | | | mouse DMPK RNA (% control) | | | | | | |
|---|---|---|---|---|---|---|---|---|---|
| pound | | Dose | | | | | | | Sciatic |
| Number | Ligand | (mg/kg) | Quad | TA | Gastroc | Heart | Diaphragm | Liver | Nerve |
| 486178 | None | 35 | 26 | 35 | 26 | 57 | 20 | 13 | 47 |
| 1590463 | BCY17873 | 3.5 | 44 | 63 | 48 | 72 | 29 | 32 | 77 |
| | BCY19405 | 3.5 | 44 | 49 | 49 | 75 | 32 | 32 | 75 |
| | BCY19406 | 3.5 | 49 | 61 | 48 | 72 | 32 | 37 | 67 |
| | BCY19407 | 3.5 | 37 | 50 | 41 | 69 | 26 | 35 | 78 |
| | BCY19409 | 3.5 | 44 | 52 | 46 | 63 | 30 | 38 | 77 |

### Body Weight

Body weights of hTFR^{KI/+} mice were measured on days 1 and 22, and the average body weight for each group is presented in the table below.

TABLE 44
Body weight (in grams)

| Compound | | Dose | Body weight (g) | |
|---|---|---|---|---|
| Number | Ligand | (mg/kg) | Day 1 | Day 22 |
| PBS | None | 0 | 30 | 30 |
| 486178 | None | 35 | 33 | 35 |
| 1590463 | BCY17873 | 3.5 | 29 | 30 |
| | BCY19405 | 3.5 | 33 | 34 |
| | BCY19406 | 3.5 | 34 | 35 |
| | BCY19407 | 3.5 | 31 | 32 |
| | BCY19409 | 3.5 | 28 | 29 |

### Example 12: Activity of Modified Oligonucleotides Conjugated to CD71 Bicycle Ligands in hTFR^{I}+ Knock in Mice, Multiple Dose

The activity and tolerability of modified oligonucleotides conjugated to CD71 bicycle ligands was tested in heterozygous hTFR^{KI/+} knock in mice (described herein above).

### Treatment

hTFR^{KI/+} knock-in mice were divided into groups of 3 mice each. Each mouse received an intravenous administration of Compound No. 1590463-BCY17901 for a total of 3 doses (on Days 1, 8, and 15) at doses indicated in the tables below. A group of 3 mice received an intravenous administration of unconjugated modified oligonucleotide, Compound No. 486178, for a total of 3 doses (on Days 1, 8, and 15). A group of 4 mice received PBS as a negative control.

### RNA Analysis

The mice were sacrificed one week post final administration (on day 22), and RNA was extracted from various muscle tissues, including quadriceps (quad), gastrocnemius (gastroc), and heart, for quantitative real time RTPCR analysis to measure amount of mouse DMPK RNA using mouse primer probe set RTS3181 (described herein above). Mouse DMPK RNA levels were normalized to mouse GAPDH. Mouse GAPDH was amplified using mouse primer probe set mGapdh_LTS00102 (described herein above). Results are presented as percent mouse DMPK RNA relative to the amount of mouse DMPK RNA in PBS treated control animals, normalized to mouse GAPDH RNA (% control). ED₅₀ values were calculated in GraphPad Prism using nonlinear fit with variable Hill slope (four parameter), Y=Bottom+(Top-Bottom)/(1+(10\hat{ }log EC50/X)\hat{ }HillSlope), with the following constraints: Top=100, Bottom=0, Hill Slope <−1.

TABLE 45
Reduction of mouse DMPK RNA in hTFR^{KI/+} knock-in mice with modified
oligonucleotides conjugated to CD71 bicycle ligands

| | | | Quad | | Gastroc | | Heart | |
|---|---|---|---|---|---|---|---|---|
| | | | DMPK | | DMPK | | DMPK | |
| Com- | | | RNA | | RNA | | RNA | |
| pound | | Dose | (% | ED₅₀ | (% | ED₅₀ | (% | ED₅₀ |
| No. | Ligand | (mg/kg) | control) | (mg/kg) | control) | (mg/kg) | control) | (mg/kg) |
| PBS | none | 0 | 100 | N/A | 100 | N/A | 100 | N/A |
| 486178 | None | 1 | 96 | 13.7 | 100 | 14.1 | 109‡ | >30 |
| | | 3.5 | 88 | | 89 | | 102 | |
| | | 10 | 58 | | 60 | | 90 | |
| | | 30 | 28 | | 27 | | 73 | |
| 1590463 | BCY17901 | 0.3 | 83 | 1.6 | 83 | 1.5 | 102 | 6.3 |
| | | 1 | 68 | | 66 | | 83 | |
| | | 3.5 | 26 | | 24 | | 57 | |
| | | 10 | 13 | | 13 | | 46 | |

‡indicates that there were fewer than 3 samples available

### Body Weight

Body weights of hTFR^{KI/+} mice were measured on days 1 and 22, and the average body weight for each group is presented in the table below.

TABLE 46
Body weight (in grams)

| Compound | | Dose | Body weight (g) | |
|---|---|---|---|---|
| No. | Ligand | (mg/kg) | Day 1 | Day 22 |
| PBS | None | 0 | 20 | 22 |
| 486178 | None | 1 | 18 | 20 |
| | | 3.5 | 20 | 21 |
| | | 10 | 19 | 21 |
| | | 30 | 19 | 21 |
| 1590463 | BCY17901 | 0.3 | 20 | 22 |
| | | 1 | 20 | 22 |
| | | 3.5 | 19 | 21 |
| | | 10 | 19 | 21 |

### Example 13: Activity of Modified Oligonucleotides Conjugated to CD71 Bicycle Ligands in Homozygous hTFR^{KI/KI} Knock in Mice, Multiple Dose

The activity and tolerability of modified oligonucleotides conjugated to CD71 bicycle ligands was tested in homozygous hTFR^{KI/KI} knock in mice Human transferrin receptor (hTFR)/CD71 knock-in mice used in these studies have the coding region of mouse exon 2 as well as the splice donor-site of mouse intron 2 replaced with the human TFR open reading frame according to NCBI transcript NM_001128148.2. Humanization of the transferrin receptor gene was done via CRISPR/Cas-9-mediated gene editing, allowing for generation of a model with constitutive expression of humanized transferrin receptor gene. Targeting strategy was based on NCBI transcripts NM_011638.4 (mouse) and NM_001128148.2 (human). A plasmid allowing expression of Cas9 mRNA, specific gRNA, and the puromycin resistance cassette; and a plasmid containing the homology regions of the mouse transferrin receptor gene, an FRT site, and the replaced human region were co-transfected into the Taconic Biosciences C57BL/6N Tac ES cell line. The homozygous humanized mice are called hTFR^{IK} knock-in mice herein. They express two copies of the humanized TFR gene under the control of the endogenous mouse promoter.

### Treatment

hTFR^{KI/KI} knock-in mice were divided into groups of 3 mice each. Each mouse received an intravenous administration of Compound No. 1590463-BCY17901 for a total of 3 doses (on Days 1, 8, and 15) at doses indicated in the tables below. A group of 3 mice received an intravenous administration of unconjugated modified oligonucleotide, Compound No. 486178, for a total of 3 doses (on Days 1, 8, and 15). A group of 4 mice received PBS as a negative control.

### RNA Analysis

The mice were sacrificed one week post final administration (on day 22), and RNA was extracted from various muscle tissues, including quadriceps (quad), gastrocnemius (gastroc), and heart for quantitative real time RTPCR analysis to measure amount of mouse DMPK RNA using mouse primer probe set RTS3181 (described herein above). Mouse DMPK RNA levels were normalized to mouse GAPDH. Mouse GAPDH was amplified using mouse primer probe set mGapdh_LTS00102 (described herein above). Results are presented as percent mouse DMPK RNA relative to the amount of mouse DMPK RNA in PBS treated control animals (as indicated in the tables below), normalized to mouse GAPDH RNA (% control). ED₅₀ values were calculated in GraphPad Prism using nonlinear fit with variable Hill slope (four parameter), Y=Bottom+(Top-Bottom)/(1+(10\hat{ }log EC50/X)\hat{ }HillSlope), with the following constraints: Top=100, Bottom=0, Hill Slope <−1.

TABLE 47
Reduction of mouse DMPK RNA in hTFR^{KI/KI} knock-in mice with modified
oligonucleotides conjugated to CD71 bicycle ligands, data relative to PBS group

| | | | Quad | | Gastroc | | Heart | |
|---|---|---|---|---|---|---|---|---|
| | | | DMPK | | DMPK | | DMPK | |
| Com- | | | RNA | | RNA | | RNA | |
| pound | | Dose | (% | ED₅₀ | (% | ED₅₀ | (% | ED₅₀ |
| No. | Ligand | (mg/kg) | control) | (mg/kg) | control) | (mg/kg) | control) | (mg/kg) |
| PBS | none | 0 | 100 | N/A | 100 | N/A | 100 | N/A |
| 486178 | None | 5 | 86 | 17.6 | 87 | 22.0 | 90 | 43.0 |
| | | 20 | 46 | | 56 | | 66 | |
| | | 50 | 18 | | 21 | | 48 | |
| 1590463 | BCY17901 | 1 | 57 | 1.2 | 66 | 1.5 | 80 | 3.1 |
| | | 3.5 | 16 | | 20 | | 40 | |
| | | 10 | 8 | | 9 | | 28 | |

### Plasma Chemistry Markers

To evaluate the effect of modified oligonucleotides on liver and kidney function, plasma levels of albumin (ALB), alanine aminotransferase (ALT), aspartate aminotransferase (AST), total bilirubin (TBIL), total Protein (PROT), Creatine (CREAT), and Creatine Kinase (CK) were measured on the day the mice were sacrificed (day 22) using an automated clinical chemistry analyzer (Hitachi Olympus AU400c, Melville, NY). The results were averaged for each group of mice and are presented in the table below.

TABLE 48
Plasma chemistry markers in hTFR^{KI/KI} knock-in mice

| | | | Plasma clinical chemistry | | | | | | |
|---|---|---|---|---|---|---|---|---|---|
| Compound | | Dose | ALB | ALT | AST | TBIL | PROT | CREAT | CK |
| No. | Ligand | (mg/kg) | (g/dL) | (U/L) | (U/L) | (mg/dL) | (g/dL) | (mg/dL) | (U/L) |
| PBS | None | 0 | 3.2 | 31 | 70 | 0.14 | 5.2 | 0.13 | 180 |
| 486178 | None | 5 | 3.2 | 41 | 80 | 0.19 | 5.2 | 0.14 | 197 |
| | | 20 | 2.9 | 30 | 57 | 0.12 | 4.8 | 0.11 | 93 |
| | | 50 | 3.3 | 30 | 111 | 0.12 | 5.0 | 0.13 | 380 |
| 1590463 | BCY17901 | 1 | 3.2 | 47 | 84 | 0.11 | 5.1 | 0.13 | 165 |
| | | 3.5 | 3.3 | 58 | 91 | 0.12 | 5.2 | 0.13 | 176 |
| | | 10 | 3.2 | 27 | 48 | 0.13 | 5.3 | 0.12 | 86 |

### Hematology Assays

Blood obtained from mice on the day the mice were sacrificed (day 22) were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), and platelets (PLT) were evaluated. The results are presented in the tables below. “N.D.” indicated values that were not determined.

TABLE 49
Hematology Parameters in hTFRK^{KI/KI} knock-in mice

| Com- | | | | | | | | | |
|---|---|---|---|---|---|---|---|---|---|
| pound | | Dose | WBC | RBC | HGB | HCT | MCV | MCH | MCHC |
| No. | Ligand | (mg/kg) | (10³/μL) | (10¹²/L) | (g/dL) | (%) | (fL) | (pg) | (g/dL) |
| PBS | None | 0 | 9 | 5 | 10 | 42 | 83 | 21 | 25 |
| 486178 | None | 5 | 9‡ | 5‡ | 11‡ | 45‡ | 85‡ | 21‡ | 25‡ |
| | | 20 | 8 | 5 | 10 | 42 | 79 | 19 | 25 |
| | | 50 | 7 | 5 | 10 | 40 | 78 | 20 | 26 |
| 1590463 | BCY17901 | 1 | 6 | 5 | 11 | 41 | 85 | 22 | 26 |
| | | 3.5 | 5 | 5 | 11 | 40 | 82 | 21 | 26 |
| | | 10 | 6 | 5 | 11 | 42 | 84 | 22 | 26 |

‡indicates that there were fewer than 3 samples available

TABLE 50
Blood cell counts in hTFR^{KI/KI} knock-in mice

| Com- | | Dose | | | | | | |
|---|---|---|---|---|---|---|---|---|
| pound | | (mg/ | NEU | LYM | MON | EOS | BAS | PLT |
| No. | Ligand | kg) | (%) | (%) | (%) | (%) | (%) | (10⁹/L) |
| PBS | None | 0 | 10 | 83 | 6 | 1.63 | 0.13 | 1851 |
| 486178 | None | 5 | 8‡ | 85‡ | 5‡ | 1.65‡ | 0.10‡ | 1654‡ |
| | | 20 | 9 | 84 | 6 | 1.43 | 0.03 | 1662‡ |
| | | 50 | 8 | 82 | 7 | 1.6 | 0.03 | 1601‡ |
| 1590463 | BCY17901 | 1 | 7 | 88 | 4 | 1.83 | 0.17 | N.D. |
| | | 3.5 | 9 | 84 | 4 | 2.4 | 0.13 | N.D. |
| | | 10 | 12 | 83 | 4 | 1.87 | 0.10 | N.D. |

‡indicates that there were fewer than 3 samples available

### Body and Organ Weights

Body weights of (hTFR^{KI/KI} mice were measured on days 1 and 22, and the average body weight for each group is presented in the table below. Liver, kidney, and spleen weights were measured on the day the mice were sacrificed (day 22), and the average organ weights for each group are presented in the tables below.

TABLE 51
Body and organ weights (in grams)

| Compound | | Dose | Body weight (g) | |
|---|---|---|---|---|
| No. | Ligand | (mg/kg) | Day 1 | Day 22 |
| PBS | None | 0 | 32 | 32 |
| 486178 | None | 5 | 30 | 30 |
| | | 20 | 30 | 30 |
| | | 50 | 33 | 32 |
| 1590463 | BCY17901 | 1 | 31 | 31 |
| | | 3.5 | 33 | 32 |
| | | 10 | 32 | 31 |

### Example 14: Design of RNAi Compounds Targeted to DMPK

Antisense strand modified oligonucleotides complementary to mouse DMPK were designed and synthesized as indicated in the table below.

TABLE 52
Design of antisense strand modified oligonucleotides targeted to mouse DMPK

| | | | SEQ |
|---|---|---|---|
| Compound | | | ID |
| No. | Chemistry Notation (5′ to 3′) | Target | NO. |
| 1653456 | vP0-TₑₛA_{fs}G_{yo}AyₒC_{yo}A_{fo}A_{yo}U_{yo}A_{fo}A_{yo}A_{yo}U_{yo}A_{yo}C_{fo}C_{yo}G_{fo}A_{yo}G_{yo}G_{ys}A_{ys}A_{y} | mouse | 173 |
| | | DMPK | |

In the table above, “vP” represents a 5′ vinyl phosphonate moiety, a subscript “f” represents a 2′-F modified nucleoside, a subscript “y” represents a 2′-OMe modified nucleoside, a subscript “e” represents a 2′ MOE modified nucleoside, a subscript “s” represents a phosphorothioate internucleoside linkage, and a subscript “o” represents a phosphodiester internucleoside linkage.

The sense oligonucleotide is complementary to the first of the 21 nucleosides of the antisense oligonucleotide (from 5′ to 3′) wherein the last two 3′-nucleosides of the antisense oligonucleotides are not paired with the sense oligonucleotide (are overhanging nucleosides). Modified oligonucleotide Compound No. 1652967 was conjugated to BCY17901 as described herein above.

TABLE 53
Design of sense strand modified oligonucleotides targeted to mouse DMPK

| | | SEQ |
|---|---|---|
| Compound | | ID |
| No. | Chemistry Notation (5′ to 3′) | NO. |
| 1547300 | C_{ys}C_{ys}U_{yo}C_{yo}G_{fo}G_{yo}U_{fo}A_{fo}U_{fo}U_{yo}U_{yo}A_{yo}U_{yo}U_{yo}G_{yo}U_{yo}C_{yo}U_{yo}A_{yo}[3nC7] | 180 |
| | [maleimidC3oyl] | |
| 1652967 | C_{ys}C_{ys}U_{yo}C_{yo}G_{fo}G_{yo}U_{fo}A_{fo}U_{fo}U_{yo}U_{yo}A_{yo}U_{yo}U_{yo}G_{yo}U_{yo}C_{yo}U_{yo}A_{yo}[3nC7][BCN] | 174 |

In the table above, a subscript “f” represents a 2′-F modified nucleoside, a subscript “y” represents a 2′-OMe modified nucleoside, a subscript “s” represents a phosphorothioate internucleoside linkage, a subscript “o” represents a phosphodiester internucleoside linkage, a “[3nC7]” represents a 3′-C7 amino modifier, a “[BCN]” indicates a (bicyclo[6.1.0]nonyne)-formyl linker, and a “[maleimidC3oyl]” indicates a maleimido propionyl linker.

TABLE 54
Design of sense strand modified
oligonucleotide conjugated to Bicycle ligand

| Conjugated | Unconjugated | |
|---|---|---|
| Compound No. | Compound No. | Ligand |
| 1678385 | 1652967 | BCY17901 |

TABLE 55
Design of RNAi compounds targeted to human/mouse DMPK

| Duplex | Antisense Strand | Sense Strand |
|---|---|---|
| Compound No. | Compound No. | Compound No. |
| 1653456:1547300 | 1653456 | 1547300 |
| 1653456:1678385 | 1653456 | 1678385 |

### Example 15: Activity of Modified Oligonucleotides Conjugated to CD71 Bicycle Ligands in hTFR^{KI/+} Knock in Mice, Multiple Dose

The activity and tolerability of modified oligonucleotides conjugated to CD71 bicycle ligands was tested in heterozygous hTFR^{KI/+} knock in mice (described herein above).

### Treatment

hTFR^{KI/+} knock-in mice were divided into groups of 3 mice each. Each mouse received an intravenous administration of Compound 1590463-BCY17901 for a total of 3 doses (on Days 1, 8, and 15) at doses indicated in the tables below. A group of 3 mice received an intravenous administration of unconjugated modified oligonucleotide, Compound No. 486178, for a total of 3 doses (on Days 1, 8, and 15). A group of 4 mice received PBS as a negative control.

### RNA Analysis

The mice were sacrificed one week post final administration (on day 22), and RNA was extracted from various muscle tissues, including quadriceps (quad), gastrocnemius (gastroc), heart, and diaphragm, and liver tissue for quantitative real time RTPCR analysis to measure amount of mouse DMPK RNA using mouse primer probe set RTS3181 (described herein above). Mouse DMPK RNA levels were normalized to mouse GAPDH. Mouse GAPDH was amplified using mouse primer probe set mGapdh_LTS00102 (described herein above). Results are presented as percent mouse DMPK RNA relative to the amount of mouse DMPK RNA in PBS treated control animals, normalized to mouse GAPDH RNA (% control). ED₅₀ values were calculated in GraphPad Prism using nonlinear fit with variable Hill slope (four parameter), Y=Bottom+(Top-Bottom)/(1+(10\hat{ }log EC50/X)\hat{ }HillSlope), with the following constraints: Top=100, Bottom=0, Hill Slope <−1.

TABLE 56
Reduction of mouse DMPK RNA in hTFR^{KI/+} knock-in mice with modified
oligonucleotides conjugated to CD71 bicycle ligands

| | | | Quad | | Gastroc | | Diaphragm | |
|---|---|---|---|---|---|---|---|---|
| | | | DMPK | | DMPK | | DMPK | |
| Com- | | | RNA | | RNA | | RNA | |
| pound | | Dose | (% | ED₅₀ | (% | ED₅₀ | (% | ED₅₀ |
| No. | Ligand | (mg/kg) | control) | (mg/kg) | control) | (mg/kg) | control) | (mg/kg) |
| 486178 | None | 3.5 | 78 | 10.5 | 82 | 11.22 | 70 | 8.30 |
| | | 20 | 32 | | 31 | | 29 | |
| | | 50 | 15 | | 16 | | 15 | |
| 1590463 | BCY17901 | 1 | 57 | 1.28 | 56 | 1.24 | 57 | 1.34 |
| | | 3.5 | 25 | | 25 | | 27 | |
| | | 10 | 12 | | 12 | | 14 | |

TABLE 57
Reduction of mouse DMPK RNA in hTFR^{KI/+} knock-in mice with
modified oligonucleotides conjugated to CD71 bicycle ligands

| | | | Heart | | Liver | |
|---|---|---|---|---|---|---|
| | | | DMPK | | DMPK | |
| Com- | | | RNA | | RNA | |
| pound | | Dose | (% | ED₅₀ | (% | ED₅₀ |
| No. | Ligand | (mg/kg) | control) | (mg/kg) | control) | (mg/kg) |
| 486178 | None | 3.5 | 90 | 47.75 | 39 | <3.5 |
| | | 20 | 67 | | 18 | |
| | | 50 | 53 | | 10 | |
| 1590463 | BCY17901 | 1 | 89 | 5.45 | 42 | <3.5 |
| | | 3.5 | 56 | | 26 | |
| | | 10 | 38 | | 14 | |

### Plasma Chemistry Markers

To evaluate the effect of modified oligonucleotides on liver and kidney function, plasma levels of albumin (ALB), alanine aminotransferase (ALT), aspartate aminotransferase (AST), total bilirubin (TBIL), blood urea nitrogen (BUN), total Protein (PROT), Creatine (CREAT), and Creatine Kinase (CK) were measured on the day the mice were sacrificed (day 22) using an automated clinical chemistry analyzer (Hitachi Olympus AU400c, Melville, NY). The results were averaged for each group of mice and are presented in the table below.

TABLE 58
Plasma chemistry markers in hTFR^{KI/+} knock-in mice

| Com- | | | Plasma clinical chemistry | | | | | | | |
|---|---|---|---|---|---|---|---|---|---|---|
| pound | | Dose | ALB | ALT | AST | TBIL | BUN | PROT | CREAT | CK |
| No. | Ligand | (mg/kg) | (g/dL) | (U/L) | (U/L) | (mg/dL) | (mg/dL) | (g/dL) | (mg/dL) | (U/L) |
| PBS | None | 0 | 3.2 | 34 | 70 | 0.14 | 23 | 5.2 | 0.16 | 99 |
| 486178 | None | 3.5 | 3.1 | 26 | 75 | 0.12 | 22 | 5.0 | 0.12 | 116 |
| | | 20 | 3.0 | 30 | 85 | 0.16 | 22 | 5.0 | 0.16 | 128 |
| | | 50 | 3.1 | 26 | 65 | 0.12 | 21 | 5.1 | 0.14 | 96 |
| 1590463 | BCY17901 | 1 | 3.1 | 55 | 123 | 0.12 | 24 | 5.1 | 0.13 | 193 |
| | | 3.5 | 2.9 | 38 | 93 | 0.11 | 20 | 4.8 | 0.14 | 167 |
| | | 10 | 3.0 | 42 | 80 | 0.16 | 23 | 4.9 | 0.13 | 88 |

### Hematology Assays

Blood obtained from mice on the day the mice were sacrificed (day 22) were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), and platelets (PLT) were evaluated. The results are presented in the tables below.

TABLE 59
Hematology Parameters in hTFR^{KI/+} knock-in mice

| Compound | | Dose | WBC | RBC | HGB | HCT | MCV | MCH | MCHC |
|---|---|---|---|---|---|---|---|---|---|
| No. | Ligand | (mg/kg) | (10³/μL) | (10¹²/L) | (g/dL) | (%) | (fL) | (pg) | (g/dL) |
| PBS | None | 0 | 4.5 | 10 | 13 | 40 | 39 | 13 | 33 |
| 486178 | None | 3.5 | 3.4 | 10 | 13 | 40 | 39 | 13 | 33 |
| | | 20 | 5.1 | 10 | 13 | 40 | 39 | 13 | 34 |
| | | 50 | 7.0 | 11 | 14 | 41 | 38 | 13 | 34 |
| 1590463 | BCY17901 | 1 | 5.9 | 10 | 13 | 41 | 39 | 13 | 33 |
| | | 3.5 | 6.0 | 11 | 14 | 40 | 37 | 13 | 34 |
| | | 10 | 5.3 | 11 | 14 | 41 | 38 | 13 | 33 |

TABLE 60
Blood cell counts in hTFR^{KI/+} knock-in mice

| Compound | | Dose | NEU | LYM | MON | EOS | BAS | PLT | RETI |
|---|---|---|---|---|---|---|---|---|---|
| No. | Ligand | (mg/kg) | (%) | (%) | (%) | (%) | (%) | (10⁹/L) | (10³/μL) |
| PBS | None | 0 | 12 | 82 | 5 | 1.33 | 0.13 | 1242 | 361 |
| 486178 | None | 3.5 | 10 | 80 | 7 | 1.73 | 0.10 | 1057 | 368 |
| | | 20 | 9 | 86 | 5 | 0.83 | 0.10 | 1151 | 411 |
| | | 50 | 10 | 81 | 8 | 1.40 | 0.10 | 929 | 413 |
| 1590463 | BCY17901 | 1 | 7 | 88 | 4 | 0.43 | 0.07 | 1495 | 464 |
| | | 3.5 | 7 | 87 | 4 | 1.37 | 0.30 | 1233 | 361 |
| | | 10 | 8 | 85 | 6 | 0.27 | 0.13 | 1183 | 421 |

### Body Weight

Body weights of hTFR^{KI/+} mice were measured on days 1 and 22, and the average body weight for each group is presented in the table below.

TABLE 61
Body weight (in grams)

| Compound | | Dose | Body weight (g) | |
|---|---|---|---|---|
| No. | Ligand | (mg/kg) | Day 1 | Day 22 |
| PBS | None | 0 | 30 | 29 |
| 486178 | None | 3.5 | 29 | 29 |
| | | 20 | 28 | 29 |
| | | 50 | 31 | 34 |
| 1590463 | BCY17901 | 1 | 31 | 32 |
| | | 3.5 | 27 | 26 |
| | | 10 | 25 | 26 |

### Example 16: Activity of RNAi Compounds Conjugated to CD71 Bicycle Ligands in hTFR^{KI/+} Knock in Mice, Multiple Dose

The activity of RNAi Compound No. 1653456:1547300 conjugated to CD71 a bicycle ligand was tested in in heterozygous hTFR^{KI/+} knock in mice (described herein above). In addition, activity of Compound No. 1653456:1547300 (described herein above) conjugated to the Fab′ fragments of OKT9 antibody (BioXCell, catalog number: BE0023) that targets human CD71 was tested.

### Treatment

hTFR^{KI/+} knock-in mice were divided into groups of 3-4 mice each. A group of 3 mice received an intravenous administration of conjugated RNAi Compound 1653456:1547300-OKT9 Fab, for a total of 3 doses (on Days 1, 8, and 15). A group of 3 mice received an intravenous administration of conjugated RNAi Compound 1653456:1678385 for a total of 3 doses (on Days 1, 8, and 15) at doses indicated in the tables below. A group of 4 mice received a subcutaneous administration of conjugated RNAi Compound 1653456:1678385 for a total of 3 doses (on Days 1, 8, and 15) at 10 mg/kg. A group of 4 mice received PBS as a negative control.

### RNA Analysis

The mice were sacrificed one week post final administration (on day 22), and RNA was extracted from various muscle tissues, including quadriceps (quad), gastrocnemius (gastroc), heart, and diaphragm, and liver tissue for quantitative real time RTPCR analysis to measure amount of mouse DMPK RNA using mouse primer probe set RTS3181 (described herein above). Mouse DMPK RNA levels were normalized to mouse GAPDH. Mouse GAPDH was amplified using mouse primer probe set mGapdh_LTS00102 (described herein above). Results are presented as percent mouse DMPK RNA relative to the amount of mouse DMPK RNA in PBS treated control animals, normalized to mouse GAPDH RNA (% control). ED₅₀ values were calculated in GraphPad Prism using nonlinear fit with variable Hill slope (four parameter), Y=Bottom+(Top-Bottom)/(1+(10\hat{ }log EC50/X)\hat{ }HillSlope), with the following constraints: Top=100, Bottom=0, Hill Slope <−1. “N.C.” indicates that a value was not calculated.

TABLE 62
Reduction of mouse DMPK RNA in hTFR^{KI/+} knock-in mice with modified oligonucleotides conjugated to
CD71 bicycle ligands

| | | | | Quad | | Gastroc | | | |
|---|---|---|---|---|---|---|---|---|---|
| | | | | DMPK | | DMPK | | Diaphragm | |
| | | | | RNA | | RNA | | DMPK | |
| | | Mode of | Dose | (% | ED₅₀ | (% | ED₅₀ | RNA | ED₅₀ |
| Compound No. | Ligand | Administration | (mg/kg) | control) | (mg/kg) | control) | (mg/kg) | (% control) | (mg/kg) |
| 1653456:1547300 | OKT9 | i.v. | 1 | 30 | <1 | 29 | <1 | 31 | <1 |
| | | | 3.5 | 22 | | 21 | | 25 | |
| | | | 10 | 20 | | 19 | | 22 | |
| 1653456:1678385 | BCY17901 | i.v. | 1 | 36 | <1 | 35 | <1 | 37 | <1 |
| | | | 3.5 | 23 | | 22 | | 28 | |
| | | | 10 | 25 | | 26 | | 27 | |
| 1653456:1678385 | BCY17901 | s.c. | 10 | 22 | N.C. | 21 | N.C. | 29 | N.C. |

TABLE 63
Reduction of mouse DMPK RNA in hTFR^{KI/+} knock-in mice with modified oligonucleotides conjugated to
CD71 bicycle ligands

| | | | | Heart | | Liver | |
|---|---|---|---|---|---|---|---|
| | | | | DMPK | | DMPK | |
| | | Mode of | Dose | RNA (% | ED₅₀ | RNA (% | ED₅₀ |
| Compound No. | Ligand | Administration | (mg/kg) | control) | (mg/kg) | control) | (mg/kg) |
| 1653456:1547300 | OKT9 | i.v. | 1 | 47 | 1.56 | 64 | 3.80 |
| | | | 3.5 | 35 | | 55 | |
| | | | 10 | 34 | | 37 | |
| 1653456:1678385 | BCY17901 | i.v. | 1 | 69 | 3.16 | 98 | >10 |
| | | | 3.5 | 41 | | 69 | |
| | | | 10 | 40 | | 76 | |
| 1653456:1678385 | BCY17901 | s.c. | 10 | 44 | N.C. | 78 | N.C. |

### Plasma Chemistry Markers

To evaluate the effect of modified oligonucleotides on liver and kidney function, plasma levels of albumin (ALB), alanine aminotransferase (ALT), aspartate aminotransferase (AST), total bilirubin (TBIL), blood urea nitrogen (BUN), total Protein (PROT), Creatine (CREAT), and Creatine Kinase (CK) were measured on the day the mice were sacrificed (day 22) using an automated clinical chemistry analyzer (Hitachi Olympus AU400c, Melville, NY). The results were averaged for each group of mice and are presented in the table below.

TABLE 64
Plasma chemistry markers in hTFR^{KI/+} knock-in mice

| | | | | Plasma clinical chemistry | | | | | | | |
|---|---|---|---|---|---|---|---|---|---|---|---|
| | | Mode of | Dose | ALB | ALT | AST | TBIL | BUN | PROT | CREAT | CK |
| Compound No. | Ligand | Administration | (mg/kg) | (g/dL) | (U/L) | (U/L) | (mg/dL) | (mg/dL) | (g/dL) | (mg/dL) | (U/L) |
| PBS | None | i.v. | 0 | 3.2 | 34 | 70 | 0.14 | 23 | 5.2 | 0.16 | 99 |
| 1653456:1547300 | OKT9 | i.v. | 1 | 3.0 | 25 | 79 | 0.14 | 20 | 5.0 | 0.11 | 124 |
| | | | 3.5 | 3.1 | 46 | 88 | 0.11 | 26 | 5.1 | 0.14 | 99 |
| | | | 10 | 3.1 | 34 | 97 | 0.13 | 18 | 5.1 | 0.15 | 192 |
| 1653456:1678385 | BCY17901 | i.v. | 1 | 3.3 | 25 | 115 | 0.12 | 22 | 5.3 | 0.12 | 247 |
| | | | 3.5 | 3.0 | 38 | 116 | 0.13 | 26 | 5.0 | 0.11 | 251 |
| | | | 10 | 3.2 | 27 | 76 | 0.14 | 21 | 5.2 | 0.13 | 119 |
| 1653456:1678385 | BCY17901 | s.c. | 10 | 3.1 | 24 | 78 | 0.14 | 19 | 4.9 | 0.13 | 117 |

### Hematology Assays

Blood obtained from mice on the day the mice were sacrificed (day 22) were sent to IDEXX BioResearch for measurement of blood cell counts. Counts taken include red blood cell (RBC) count, white blood cell (WBC) count, hemoglobin (HGB), hematocrit (HCT), Mean corpuscular volume (MCV), mean corpuscular hemoglobin (MCH), and mean corpuscular hemoglobin concentration (MCHC). Individual white blood cell counts, such as that of monocytes (MON), neutrophils (NEU), lymphocytes (LYM), eosinophils (EOS), basophils (BAS), and platelets (PLT) were evaluated. The results are presented in the tables below.

TABLE 65
Hematology Parameters in hTFR^{KI/+} knock-in mice

| | | Mode of | Dose | WBC | RBC | HGB | HCT | MCV | MCH | MCHC |
|---|---|---|---|---|---|---|---|---|---|---|
| Compound No. | Ligand | Administration | (mg/kg) | 10³/μL) | (10¹²/L) | (g/dL) | (%) | (fL) | (pg) | (g/dL) |
| PBS | None | i.v. | 0 | 4.5 | 10 | 13 | 40 | 39 | 13 | 33 |
| 1653456:1547300 | OKT9 | i.v. | 1 | 4.4 | 11 | 14 | 40 | 38 | 13 | 34 |
| | | | 3.5 | 6.9 | 11 | 14 | 40 | 37 | 13 | 34 |
| | | | 10 | 4.1 | 11 | 13 | 40 | 37 | 13 | 34 |
| 1653456:1678385 | BCY17901 | i.v. | 1 | 5.3 | 11 | 13 | 41 | 38 | 13 | 33 |
| | | | 3.5 | 5.4 | 10 | 13 | 40 | 38 | 13 | 33 |
| | | | 10 | 4.5 | 11 | 14 | 41 | 37 | 13 | 34 |
| 1653456:1678385 | BCY17901 | s.c. | 10 | 4.8 | 10 | 13 | 40 | 39 | 12 | 33 |

TABLE 66
Blood cell counts in hTFR^{KI/+} knock-in mice

| | | Mode | | | | | | | | |
|---|---|---|---|---|---|---|---|---|---|---|
| | | of | Dose | NEU | LYM | MON | EOS | BAS | PLT | RETI |
| Compound No. | Ligand | Admin. | (mg/kg) | (%) | (%) | (%) | (%) | (%) | (10⁹/L) | (10³/μL) |
| PBS | None | i.v. | 0 | 12.0 | 82 | 5 | 1.33 | 0.13 | 1242 | 361 |
| 1653456:1547300 | OKT9 | i.v. | 1 | 10.8 | 82 | 6 | 0.33 | 0.07 | 1065 | 430 |
| | | | 3.5 | 9.4 | 83 | 6 | 1.27 | 0.13 | 1117 | 412 |
| | | | 10 | 10.0 | 82 | 7 | 1.20 | 0.23 | 1268 | 430 |
| 1653456:1678385 | BCY17901 | i.v. | 1 | 6.8 | 87 | 5 | 1.13 | 0.10 | 1043 | 393 |
| | | | 3.5 | 9.9 | 86 | 4 | 0.43 | 0.00 | 887 | 367 |
| | | | 10 | 9.1 | 85 | 5 | 1.03 | 0.17 | 1090 | 396 |
| 1653456:1678385 | BCY17901 | s.c. | 10 | 5.8 | 88 | 5 | 0.63 | 0.15 | 1368 | 389 |

### Body Weight

Body weights of hTFR^{KI/+} mice were measured on days 1 and 22, and the average body weight for each group is presented in the table below.

TABLE 67
Body weight (in grams)

| | | | | Body weight | |
|---|---|---|---|---|---|
| | | Mode of | Dose | (g) | |
| Compound No. | Ligand | Administration | (mg/kg) | Day 1 | Day 22 |
| PBS | None | i.v. | 0 | 30 | 29 |
| 1653456:1547300 | OKT9 Fab′ | i.v. | 1 | 30 | 29 |
| | | | 3.5 | 34 | 33 |
| | | | 10 | 29 | 29 |
| 1653456:1678385 | BCY17901 | i.v. | 1 | 28 | 28 |
| | | | 3.5 | 32 | 31 |
| | | | 10 | 26 | 26 |
| 1653456:1678385 | BCY17901 | s.c. | 10 | 28 | 28 |

### Example 17: Design of RNAi Compounds Targeted to DMPK Conjugated to CD71 Bicycle Ligands

A sense modified oligonucleotide was designed and synthesized as indicated in the table below. The sense oligonucleotide is complementary to the first of the 21 nucleosides (from 5′ to 3′) of the antisense oligonucleotide Compound No. 1653456 (described herein above), wherein the last two 3′-nucleosides of the antisense oligonucleotides are not paired with the sense oligonucleotide (are overhanging nucleosides).

TABLE 68
Design of sense strand modified oligonucleotides targeted to mouse DMPK

| | | SEQ |
|---|---|---|
| Compound | | ID |
| No. | Chemistry Notation (5′ to 3′) | NO. |
| 1709195 | [BCN][nC60]ₒC_{ys}C_{ys}U_{yo}C_{yo}G_{fo}G_{yo}U_{fo}A_{fo}U_{fo}U_{yo}U_{yo}A_{yo}U_{yo}U_{yo}G_{yo}U_{yo}C_{ys}U_{ys}A_{y} | 189 |

In the table above, a subscript “f” represents a 2′-F modified nucleoside, a subscript “y” represents a 2′-OMe modified nucleoside, a subscript “s” represents a phosphorothioate internucleoside linkage, a subscript “o” represents a phosphodiester internucleoside linkage, a “[nC6o]” indicates a 6-aminohexanol linker, and a “[BCN]” indicates a (bicyclo[6.1.0]nonyne)-formyl linker.

Modified oligonucleotide Compound Nos. 1709195 and 1590463 were further conjugated to BCY17901 as described in the table below.

TABLE 69
Design of sense strand modified
oligonucleotide conjugated to Bicycle ligand

| Conjugated | Unconjugated | |
|---|---|---|
| Compound No. | Compound No. | Ligand |
| 1709196 | 1709195 | BCY17901 |

TABLE 70
Design of RNAi compounds targeted to human/mouse DMPK

| Duplex | Antisense Strand | Sense Strand |
|---|---|---|
| Compound No. | Compound No. | Compound No. |
| 1710872 | 1653456 | 1709196 |
| 1678710 | 1653456 | 1678385 |

### Example 18: Activity of Modified Oligonucleotides Conjugated to CD71 Bicycle Ligands in Heterozygous hTFR^{KI/+} Knock in Mice, Multiple Dose

The activity and tolerability of modified oligonucleotides conjugated to CD71 bicycle ligands was tested in in heterozygous hTFR^{KI/+} knock in mice (described herein above).

### Treatment

hTFR^{KI/+} knock in mice were divided into groups of 3 mice each. Each mouse received an intravenous (i.v.) administration or subcutaneous (s.c.) administration of conjugated modified oligonucleotide for a total of 3 doses (on Days 1, 8, and 15) at doses indicated in the tables below. A group of 3 mice received a subcutaneous administration of unconjugated modified oligonucleotide, Compound No. 486178, for a total of 3 doses (on Days 1, 8, and 15). A group of 4 mice received PBS as a negative control.

### RNA Analysis

The mice were sacrificed one week post final administration (on day 22), and RNA was extracted from various muscle tissues (including quadriceps (quad), gastrocnemius (gastroc), heart, liver) for quantitative real time RTPCR analysis to measure amount of mouse DMPK RNA using mouse primer probe set RTS3181 (described herein above). Mouse DMPK RNA levels were normalized to mouse GAPDH. Mouse GAPDH was amplified using mouse primer probe set mGapdh_LTS00102 (described herein above). Results are presented as percent mouse DMPK RNA relative to the amount of mouse DMPK in PBS treated control animals, normalized to mouse GAPDH RNA (% control). ED₅₀ values were calculated in GraphPad Prism using nonlinear fit with variable Hill slope (four parameter), Y=Bottom+(Top-Bottom)/(1+(10\hat{ }log EC50/X)\hat{ }HillSlope), with the following constraints: Top=100, Bottom=0, Hillslope <−1.

TABLE 71
Reduction of mouse DMPK RNA in hTFR^{KI/+} knock in mice with modified oligonucleotides conjugated to
CD71 bicycle ligands

| | | | | | Quad | | Liver | |
|---|---|---|---|---|---|---|---|---|
| | | | | | DMPK | | DMPK | |
| Conjugated | Unconjugated | | | | RNA | | RNA | |
| Compound | Compound | | Route of | Dose | (% | ED₅₀ | (% | ED₅₀ |
| No. | No. | Ligand | Administration | (mg/kg) | control) | (mg/kg) | control) | (mg/kg) |
| N/A | 486178 | None | s.c. | 3.5 | 92 | 16.2 | 34 | 2.0 |
| | | | | 20 | 40 | | 14 | |
| | | | | 50 | 19 | | 9 | |
| 1666846 | 1590463 | BCY17901 | s.c. | 1 | 76 | 3.3 | 48 | 1.3 |
| | | | | 3.5 | 53 | | 34 | |
| | | | | 10 | 18 | | 20 | |
| | | | i.v. | 1 | 62 | 1.4 | 50 | 1.1 |
| | | | | 3.5 | 22 | | 25 | |
| | | | | 10 | 13 | | 20 | |

TABLE 72
Reduction of mouse DMPK RNA in hTFR^{KI/+} knock in mice with modified oligonucleotides conjugated to
CD71 bicycle ligands

| | | | | | Gastroc | | Heart | |
|---|---|---|---|---|---|---|---|---|
| | | | | | DMPK | | DMPK | |
| Conjugated | Unconjugated | | | | RNA | | RNA | |
| Compound | Compound | | Route of | Dose | (% | ED₅₀ | (% | ED₅₀ |
| No. | No. | Ligand | Administration | (mg/kg) | control) | (mg/kg) | control) | (mg/kg) |
| N/A | 486178 | None | s.c. | 3.5 | 92 | 15.7 | 101 | >50 |
| | | | | 20 | 39 | | 75 | |
| | | | | 50 | 19 | | 55 | |
| 1666846 | 1590463 | BCY17901 | s.c. | 1 | 73 | 2.7 | 98 | 9.9 |
| | | | | 3.5 | 47 | | 84 | |
| | | | | 10 | 16 | | 50 | |
| | | | i.v. | 1 | 54 | 1.1 | 85 | 6.6 |
| | | | | 3.5 | 22 | | 53 | |
| | | | | 10 | 13 | | 52 | |

### Body Weights

Body weights of hTFR^{KI/+} mice were measured on days 1 and 22, and the average body weight for each group is presented in the table below.

TABLE 73
Body weights (in grams)

| Conjugated | Unconjugated | | | Dose | Body weight | |
|---|---|---|---|---|---|---|
| Compound | Compound | | Route of | (mg/ | (g) | |
| No. | No. | Ligand | Administration | kg) | Day 1 | Day 22 |
| N/A | PBS | None | s.c. | 0 | 24 | 24 |
| N/A | 486178 | None | s.c. | 3.5 | 28 | 28 |
| | | | | 20 | 26 | 26 |
| | | | | 50 | 26 | 27 |
| 1666846 | 1590463 | BCY17901 | s.c. | 1 | 23 | 23 |
| | | | | 3.5 | 24 | 24 |
| | | | | 10 | 26 | 26 |
| | | | i.v. | 1 | 30 | 30 |
| | | | | 3.5 | 30 | 28 |
| | | | | 10 | 27 | 27 |

### Example 19: Activity and Tolerability of RNAi Compounds Conjugated to CD71 Bicycle Ligands in Heterozygous hTFR^{KI/+} Knock in Mice, Multiple Dose

The activity and tolerability of RNAi compounds conjugated to CD71 bicycle ligands was tested in in heterozygous hTFR^{KI/+} knock in mice (described herein above).

### Treatment

hTFR^{KI/+} knock in mice were divided into groups of 3 mice each. Each mouse received an intravenous (i.v.) administration or subcutaneous (s.c.) administration of conjugated RNAi compound for a total of 3 doses (on Days 1, 8, and 15) at doses indicated in the tables below. A group of 4 mice received PBS as a negative control.

### RNA Analysis

The mice were sacrificed one week post final administration (on day 22), and RNA was extracted from various muscle tissues (including quadriceps (quad), gastrocnemius (gastroc), heart, liver) for quantitative real time RTPCR analysis to measure amount of mouse DMPK RNA using mouse primer probe set RTS3181 (described herein above). Mouse DMPK RNA levels were normalized to mouse GAPDH. Mouse GAPDH was amplified using mouse primer probe set mGapdh_LTS00102 (described herein above). Results are presented as percent mouse DMPK RNA relative to the amount of mouse DMPK in PBS treated control animals, normalized to mouse GAPDH RNA (% control). ED₅₀ values were calculated in GraphPad Prism using nonlinear fit with variable Hill slope (four parameter), Y=Bottom+(Top-Bottom)/(1+(10\hat{ }log EC50/X)\hat{ }HillSlope), with the following constraints: Top=100, Bottom=0, Hillslope <−1.

TABLE 74
Reduction of mouse DMPK RNA in hTFR^{KI/+} knock in mice with RNAi compounds conjugated to CD71
bicycle ligands

| | | | | | Quad | | Liver | |
|---|---|---|---|---|---|---|---|---|
| | | | | | DMPK | | DMPK | |
| Conjugated | | | | | RNA | | RNA | |
| Compound | Unconjugated | | Route of | Dose | (% | ED₅₀ | (% | ED₅₀ |
| No. | Compound No. | Ligand | Administration | (mg/kg) | control) | (mg/kg) | control) | (mg/kg) |
| 1678710 | 1653456: | BCY17901 | s.c. | 0.1 | 85 | 0.7 | 107 | >10 |
| | 1652967 | | | 0.3 | 65 | | 106 | |
| | | | | 1 | 41 | | 87 | |
| | | | | 3.5 | 24‡ | | 106 | |
| | | | | 10 | 22 | | 94 | |
| | | | i.v. | 0.1 | 70 | 0.5 | 96 | >10 |
| | | | | 0.3 | 55 | | 125 | |
| | | | | 1 | 36 | | 88 | |
| | | | | 3.5 | 28 | | 93 | |
| | | | | 10 | 25 | | 88 | |
| 1710872 | 1653456:1709195 | BCY17901 | i.v. | 0.1 | 81 | 0.5 | 106 | >10 |
| | | | | 0.3 | 57 | | 118 | |
| | | | | 1 | 31 | | 112 | |
| | | | | 3.5 | 27 | | 77 | |
| | | | | 10 | 21 | | 92 | |

‡indicates that there were fewer than 3 samples available

TABLE 75
Reduction of mouse DMPK RNA in hTFR^{KI/+} knock in mice with modified oligonucleotides
conjugated to CD71 bicycle ligands

| | | | | | Gastroc | | Heart | |
|---|---|---|---|---|---|---|---|---|
| | | | | | DMPK | | DMPK | |
| Conjugated | | | | | RNA | | RNA | |
| Compound | Unconjugated | | Route of | Dose | (% | ED₅₀ | (% | ED₅₀ |
| No. | Compound No. | Ligand | Administration | (mg/kg) | control) | (mg/kg) | control) | (mg/kg) |
| 1678710 | 1653456: | BCY17901 | s.c. | 0.1 | 77 | 0.6 | 98 | 5.5 |
| | 1652967 | | | 0.3 | 63 | | 85 | |
| | | | | 1 | 37 | | 76 | |
| | | | | 3.5 | 22 | | 55 | |
| | | | | 10 | 21 | | 49 | |
| | | | i.v. | 0.1 | 66 | 0.4 | 90 | 3.9 |
| | | | | 0.3 | 53 | | 91 | |
| | | | | 1 | 34 | | 66 | |
| | | | | 3.5 | 27 | | 50 | |
| | | | | 10 | 23 | | 43 | |
| 1710872 | 1653456:1709195 | BCY17901 | i.v. | 0.1 | 68 | 0.4 | 98 | 4.9 |
| | | | | 0.3 | 52 | | 89 | |
| | | | | 1 | 29 | | 78 | |
| | | | | 3.5 | 25 | | 49 | |
| | | | | 10 | 20 | | 48 | |

### Plasma Chemistry Markers

To evaluate the effect of modified oligonucleotides on liver and kidney function, plasma levels of albumin (ALB), alanine aminotransferase (ALT), aspartate aminotransferase (AST), total bilirubin (TBIL), total Protein (PROT), Creatine (CREAT), Iron, and Creatine Kinase (CK) were measured on the day the mice were sacrificed (day 18) using an automated clinical chemistry analyzer (Hitachi Olympus AU400c, Melville, NY). The results were averaged for each group of mice and are presented in the table below.

TABLE 76
Plasma chemistry markers in hTFR^{KI/+} knock in mice

| Conjugated | | | Route of | Dose | | | | | | | IRON | |
|---|---|---|---|---|---|---|---|---|---|---|---|---|
| Compound | Unconjugated | | Admin- | (mg/ | ALB | ALT | AST | TBIL | BUN | CREAT | (μg/ | CK |
| No. | Compound No. | Ligand | istration | kg) | (g/dL) | (U/L) | (U/L) | (mg/dL) | (mg/dL) | (mg/dL) | dL) | (U/L) |
| N/A | PBS | None | s.c. | 0 | 3.35 | 40 | 95 | 0.13 | 24 | 0.17 | 274 | 206 |
| 1678710 | 1653456: | BCY17901 | s.c. | 10 | 3.11 | 20 | 66 | 0.10 | 20 | 0.16 | 244 | 175 |
| | 1652967 | | i.v. | 10 | 3.09 | 47 | 119 | 0.12 | 25 | 0.17 | 253 | 332 |
| 1710872 | 1653456: | BCY17901 | i.v. | 10 | 3.13 | 91 | 115 | 0.11 | 18 | 0.18 | 259 | 125 |
| | 1709195 | | | | | | | | | | | |

### Body and Organ Weights

Body weights of hTFR^{KI/+} mice were measured on days 1 and 22, and the average body weight for each group is presented in the table below. Liver, kidney, and spleen weights were measured on the day the mice were sacrificed (day 22), and the average organ weights for each group are presented in the tables below. “N.D.” indicates the data was not determined.

TABLE 77
Body and organ weights (in grams)

| Conjugated | | | Route of | Dose | Body | | | | |
|---|---|---|---|---|---|---|---|---|---|
| Compound | Unconjugated | | Adminis- | (mg/ | weight (g) | | Organ weight (g) | | |
| No. | Compound No. | Ligand | tration | kg) | Day 1 | Day 22 | Liver | Kidney | Spleen |
| N/A | PBS | None | s.c. | 0 | 24 | 24 | 1.21 | 0.31 | 0.10 |
| 1678710 | 1653456: | BCY17901 | s.c. | 0.1 | 23 | 24 | N.D. | N.D. | N.D. |
| | 1652967 | | | 0.3 | 28 | 27 | N.D. | N.D. | N.D. |
| | | | | 1 | 24 | 24 | N.D. | N.D. | N.D. |
| | | | | 3.5 | 24 | 25 | N.D. | N.D. | N.D. |
| | | | | 10 | 23 | 22 | 0.92 | 0.27 | 0.08 |
| | | | i.v. | 0.1 | 27 | 26 | N.D. | N.D. | N.D. |
| | | | | 0.3 | 25 | 26 | N.D. | N.D. | N.D. |
| | | | | 1 | 23 | 23 | N.D. | N.D. | N.D. |
| | | | | 3.5 | 26 | 25 | N.D. | N.D. | N.D. |
| | | | | 10 | 23 | 24 | 1.14 | 0.29 | 0.09 |
| 1710872 | 1653456: | BCY17901 | i.v. | 0.1 | 26 | 26 | N.D. | N.D. | N.D. |
| | 1709195 | | | 0.3 | 25 | 26 | N.D. | N.D. | N.D. |
| | | | | 1 | 24 | 23 | N.D. | N.D. | N.D. |
| | | | | 3.5 | 25 | 25 | N.D. | N.D. | N.D. |
| | | | | 10 | 25 | 25 | 1.05 | 0.29 | 0.07 |

### Example 20: Design of Modified Oligonucleotides Targeted to MALAT Conjugated to CD71 Bicycle Ligands

Modified oligonucleotides complementary to mouse MALAT were designed (as indicated in the table below) and synthesized. The compound in the table below has a modification on the 5′ end to allow conjugation to a bicycle ligand.

TABLE 78
Design of modified oligonucleotides complementary to mouse MALAT

| | | SEQ |
|---|---|---|
| Compound | | ID |
| No. | Chemistry Notation (5′ to 3′) | NO. |
| 1694083 | [BCN][nC60]ₒAₖₛGₖₛTₖₛA_{ds}^{m}C_{ds}T_{ds}A_{ds}T_{ds}A_{ds}G_{ds}^{m}C_{ds}A_{ds}T_{ds}^{m}CₖₛTₖₛGₖ | 184 |

In the table above, a subscript “k” represents a cEt nucleoside, a subscript “d” represents a stereo-standard DNA nucleoside, a subscript “s” indicates a phosphorothioate internucleoside linkage, a subscript “o” indicates a phosphodiester internucleoside linkage, a superscript “m” before a C represents a 5-methyl cytosine, a “[nC6o]” indicates a 6-aminohexanol linker, and a “[BCN]” indicates a (bicyclo[6.1.0]nonyne)-formyl linker.

The modified oligonucleotide was further conjugated to BCY17901 as described in the table below.

TABLE 79
Design of modified oligonucleotide
conjugated to Bicycle ligand

| Conjugated | Unconjugated | |
|---|---|---|
| Compound No. | Compound No. | Ligand |
| 1694082 | 1694083 | BCY17901 |

### Example 21: Design of RNAi Compounds Targeted to HPRT Conjugated to CD71 Bicycle Ligands

Antisense strand modified oligonucleotides complementary to mouse HPRT were designed and synthesized as indicated in the table below.

TABLE 80
Design of antisense strand modified oligonucleotides targeted to human/mouse HPRT1

| Compound | | SEQ |
|---|---|---|
| No. | Chemistry Notation (5′ to 3′) | ID NO. |
| 1586322 | vP- | 191 |
| | TₑₛU_{fs}A_{yo}A_{yo}A_{yo}A_{fo}U_{yo}C_{yo}U_{yo}A_{yo}C_{yo}A_{yo}G_{yo}U_{fo}C_{yo}A_{fo}U_{yo}A_{yo}G_{yo}G_{yo}A_{ys}A_{ys}U_{y} | |

In the table above, “vP” represents a 5′ vinyl phosphonate moiety, a subscript “f” represents a 2′-F modified nucleoside, a subscript “y” represents a 2′-OMe modified nucleoside, a subscript “e” represents a 2′ MOE modified nucleoside, a subscript “s” represents a phosphorothioate internucleoside linkage, and a subscript “o” represents a phosphodiester internucleoside linkage.

A sense modified oligonucleotide was designed and synthesized as indicated in the table below. The sense oligonucleotide is complementary to the first of the 21 nucleosides (from 5′ to 3′) of the antisense oligonucleotide wherein the last two 3′-nucleosides of the antisense oligonucleotides are not paired with the sense oligonucleotide (are overhanging nucleosides). The sense modified oligonucleotide has a modification on the 3′ end to allow conjugation to a bicycle ligand.

TABLE 81
Design of sense strand modified oligonucleotides targeted to mouse HPRT

| | | SEQ |
|---|---|---|
| Compound | | ID |
| No. | Chemistry Notation (5′ to 3′) | NO. |
| 1653454 | U_{ys}C_{ys}C_{yo}U_{yo}A_{yo}U_{yo}G_{fo}A_{yo}C_{fo}U_{fo}G_{fo}U_{yo}A_{yo}G_{yo}A_{yo}U_{yo}U_{yo}U_{yo}U_{yo}A_{yo}A_{yo}[3nC7] | 190 |
| | [BCN] | |

In the table above, a subscript “f” represents a 2′-F modified nucleoside, a subscript “y” represents a 2′-OMe modified nucleoside, a subscript “s” represents a phosphorothioate internucleoside linkage, a subscript “o” represents a phosphodiester internucleoside linkage, a “[BCN]” indicates a (bicyclo[6.1.0]nonyne)-formyl linker, and a “[3nC7]” represents a 3′-C7 amino modifier having the following structure:

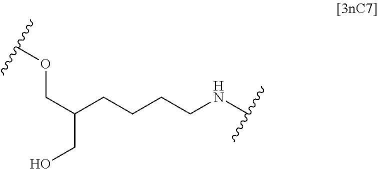 [SMILES: *NCCCCC(CO)CO*]

Sense modified oligonucleotide Compound 1653454 was further conjugated to BCY17901 as described in the table below.

TABLE 82
Design of sense strand modified
oligonucleotide conjugated to Bicycle ligand

| Conjugated | Unconjugated | |
|---|---|---|
| Compound No. | Compound No. | Ligand |
| 1694090 | 1653454 | BCY17901 |

TABLE 83
Design of RNAi compounds targeted to mouse HPRT

| Duplex | Antisense Strand | Sense Strand |
|---|---|---|
| Compound No. | Compound No. | Compound No. |
| 1694595 | 1586322 | 1694090 |

### Example 22: Effect of Oligomeric Agents Comprising CD71 Bicycle Ligands in Cynomolgus Monkeys

Cynomolgus monkeys were treated with modified oligonucleotides conjugated to CD71 bicycle ligands or RNAi compounds conjugated to CD71 bicycle ligands selected from studies described in the Examples above.

### Treatment

Prior to the study, the monkeys were kept in quarantine during which the animals were observed daily for general health. The monkeys were 2-4 years old and weighed 2-4 kg. Female cynomolgus monkeys were divided into 4 groups of 3 monkeys each. Each monkey received an intravenous (i.v.) infusion (1 hr) of conjugated modified oligonucleotide or conjugated RNAi compound via a percutaneously placed catheter. A new sterile indwelling catheter was placed before each dose. The monkeys were dosed with 10 mL/kg once per week for a total of 3 doses (on days 1, 8, 15) at 25 mg/kg.

During the study period, the monkeys were observed at least once daily for signs of illness or distress. Any animal experiencing more than momentary or slight pain or distress due to the treatment, injury or illness was treated by the veterinary staff with approved analgesics or agents to relieve the pain after consultation with the Study Director. Any animal in poor health or in a possible moribund condition was identified for further monitoring and possible euthanasia as soon as possible with attending veterinarian consultation. Scheduled euthanasia of the animals was conducted on day 28, approximately 13 days after the last dose, by exsanguination while under deep anesthesia. The protocols described in the Example were approved by the Institutional Animal Care and Use Committee (IACUC).

### RNA Analysis

To evaluate the effect of conjugated modified oligonucleotides or conjugated RNAi compounds on target RNA expression levels, cross-sectional slices of skeletal muscle tissues (quadriceps (quad), gastrocnemius (gastroc), tibialis anterior (TA), diaphragm, soleus) and heart tissue were collected from all animals. Approximately 8 mm² or 300 mg of liver tissue was be collected from all study animals and placed in 2 mL screw-cap tubes (150 mg x 2 tubes).

The monkeys were sacrificed two weeks post final administration (on day 28), and RNA was extracted from muscle tissues (including quadriceps (quad), diaphragm, soleus, tibialis anterior (TA), gastrocnemius (gastroc), heart), and liver for quantitative real time RTPCR analysis to measure amount of target RNA.

MALAT RNA levels were measured using NHP primer probe set MALATI_LTS01104 (forward sequence AAGGAGTGTACCGCTGTACTGTTG, designated herein as SEQ ID NO: 247; reverse sequence CCAAAGCTGCACTGTGCTGTA, designated herein as SEQ ID NO: 248; probe sequence ACACCTTCAGGGACTGGAGCTGCTTTTATC, designated herein as SEQ ID NO: 249). Monkey MALAT RNA levels were normalized to total RNA content, as measured by RIBOGREEN®. Results are presented as percent monkey MALAT RNA relative to the amount of monkey MALAT RNA in all groups not treated with the MALAT-targeting conjugated modified oligonucleotide. DMPK RNA levels were measured using NHP primer probe set DMPK_RTS4447 (forward sequence AGCCTGAGCCGGGAGATG, designated herein as SEQ ID NO: 250; reverse sequence GCGTAGTTGACTGGCAAAGTT, designated herein as SEQ ID NO: 251; probe sequence AGGCCATCCGCATGGCCAACC, designated herein as SEQ ID NO: 252). Monkey DMPK RNA levels were normalized to total RNA content, as measured by RIBOGREEN®. Results are presented as percent monkey DMPK RNA relative to the amount of monkey DMPK RNA in all groups not treated with the DMPK-targeting conjugated modified oligonucleotide or conjugated RNAi compound. HPRT RNA levels were measured using NHP primer probe set Rh02800695_ml (Thermofisher). Monkey HPRT RNA levels were normalized to total RNA content, as measured by RIBOGREEN®. Results are presented as percent monkey HPRT RNA relative to the amount of monkey HPRT RNA in all groups not treated with the HPRT-targeting conjugated RNAi compound.

TABLE 84
Reduction of target RNA in cynomolgus monkeys with oligomeric agents comprising CD71 bicycle ligands

| Conjugated | | | | | | | | | | |
|---|---|---|---|---|---|---|---|---|---|---|
| Compound | Unconjugated | | | Target RNA (% control) | | | | | | |
| No. | Compound No. | Ligand | Target | Quad | Heart | Diaphragm | Soleus | TA | Gastroc | Liver |
| 1694082 | 1694083 | BCY17901 | MALAT | 29 | 37 | 48 | 46 | 47 | 46 | 32 |
| 1666846 | 1590463 | BCY17901 | DMPK | 33 | 59 | 31 | 36 | 25 | 31 | 57 |
| 1678710 | 1653456:1652967 | BCY17901 | DMPK | 65 | 85 | 51 | 42 | 36 | 41 | 71 |
| 1694595 | 1586322:1653454 | BCY17901 | HPRT | 14 | 25 | 25 | 17 | 21 | 20 | 89 |

### Example 23: Design of Modified Oligonucleotides Targeted to DMPK Conjugated to CD71 Bicycle Ligands

Modified oligonucleotides complementary to mouse DMPK were designed (as indicated in the table below) and synthesized. The compound in the table below has a modification on the 5′ end to allow conjugation to a bicycle ligand.

TABLE 85
Design of modified oligonucleotides complementary to mouse DMPK

| | | SEQ |
|---|---|---|
| Compound | | ID |
| No. | Chemistry Notation (5′ to 3′) | NO. |
| 1730822 | [BCN][nC60]ₒAₖₛ^{m}CₖₒAₖₒA_{ds}T_{ds}A_{ds}A_{ds}A_{ds}T_{ds}A_{ds}^{m}C_{ds}^{m}C_{ds}G_{ds}AₖₒGₖₛGₖ | 186 |
| 1730823 | [BCN][nC60]ₒAₖₛ^{m}CₖₛAₖₛA_{dz}T_{dz}A_{dz}A_{dz}A_{ds}T_{ds}A_{ds}^{m}C_{ds}^{m}C_{ds}G_{ds}AₖₛGₖₛGₖ | 187 |
| 1730824 | [BCN][nC60]ₒAₖₛ^{m}CₖₒAₖₒA_{dz}T_{dz}A_{dz}A_{dz}A_{ds}T_{ds}A_{ds}^{m}C_{ds}^{m}C_{ds}G_{ds}AₖₒGₖₛGₖ | 188 |

In the table above, a subscript “k” represents a cEt nucleoside, a subscript “d” represents a stereo-standard DNA nucleoside, a subscript “z” represents a mesyl phosphoramidate internucleoside linkage, a subscript “s” indicates a phosphorothioate internucleoside linkage, a subscript “o” indicates a phosphodiester internucleoside linkage, a superscript “m” before a C represents a 5-methyl cytosine, a “[nC6o]” indicates a 6-aminohexanol linker, and a “[BCN]” indicates a (bicyclo[6.1.0]nonyne)-formyl linker.

The modified oligonucleotide was further conjugated to BCY17901 as described in the table below.

TABLE 86
Design of modified oligonucleotides
conjugated to Bicycle ligand

| Conjugated | Unconjugated | |
|---|---|---|
| Compound No. | Compound No. | Ligand |
| 1731582 | 1730822 | BCY17901 |
| 1731583 | 1730823 | BCY17901 |
| 1731584 | 1730824 | BCY17901 |

### Example 24: Activity of Modified Oligonucleotides Conjugated to CD71 Bicycle Ligands in Heterozygous hTFR^{KI/+} Knock in Mice

The activity and tolerability of modified oligonucleotides conjugated to CD71 bicycle ligands was tested in in heterozygous hTFR^{KI/+} knock in mice (described herein above).

### Treatment

hTFR^{KI/+} knock in mice were divided into groups of 4 mice each. Each mouse received an intravenous (i.v.) administration or subcutaneous (s.c.) administration of 3.5 mg/kg conjugated modified oligonucleotide for a total of 3 doses (on Days 1, 8, and 15), as indicated in the tables below. A group of 4 mice received PBS as a negative control.

### RNA Analysis

The mice were sacrificed one week post final administration for conjugated modified oligonucleotide groups on day 22. RNA was extracted from various muscle tissues (including quadriceps (quad) amd gastrocnemius (gastric)) for quantitative real time RTPCR analysis to measure amount of mouse DMPK RNA using mouse primer probe set RTS3181 (described herein above). Mouse DMPK RNA levels were normalized to mouse GAPDH. Mouse GAPDH was amplified using mouse primer probe set mGapdh_LTS00102 (described herein above). Results are presented as percent mouse DMPK RNA relative to the amount of mouse DMPK in s.c-PBS treated control animals, normalized to mouse GAPDH RNA (% control).

TABLE 89
Reduction of mouse DMPK RNA in hTFR^{KI/+} knock in mice
with modified oligonucleotides conjugated to CD71 bicycle ligands

| | Unconjugated | | Route of | DMPK RNA | |
|---|---|---|---|---|---|
| Conjugated | Compound | | Admini- | (% control) | |
| Compound No. | No. | Ligand | stration | Quad | Gastroc |
| 1666846 | 1590463 | BCY17901 | s.c. | 48 | 47 |
| | | | i.v. | 22 | 19 |
| 1731582 | 1730822 | BCY17901 | s.c. | 32 | 28 |
| | | | i.v. | 15 | 15 |
| 1731583 | 1730823 | BCY17901 | s.c. | 38 | 37 |
| | | | i.v. | 23 | 23 |
| 1731584 | 1730824 | BCY17901 | s.c. | 28 | 27 |
| | | | i.v. | 21 | 20 |

### Example 25: Design and Activity of RNAi Compounds Conjugated to CD71 Bicycle Ligands

TABLE 90
Bicycle ligands

| Bicycle Ligand | | N-terminal | SEQ ID |
|---|---|---|---|
| ID | Sequence (N to C) | modification | NO: |
| BCY21757 | CP[HyP]DAYLGC[tBuGly]SYCEPWK | acetyl | 245 |
| BCY21758 | CP[HyP]DAYLGC[tBuGly]SYCEPWC | acetyl | 246 |

Various sense modified oligonucleotides are designed. The sense oligonucleotides are complementary to the first of the 21 nucleosides (from 5′ to 3′) of the antisense oligonucleotides, wherein the last two 3′-nucleosides of the antisense oligonucleotides are not paired with the sense oligonucleotide (are overhanging nucleosides). The sense modified oligonucleotides are conjugated on the 5′ end to a bicycle ligand to form the following Conjugates:

TABLE 91
Conjugates with various linkers

| Bicycle Ligand | | |
|---|---|---|
| ID | Linker Name | Conjugate Structure |
| BCY21758 | Maleimide | 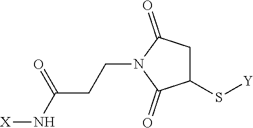 |
| BCY21757 | Bis-amide | 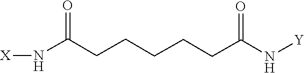 |
| BCY21757 | ketoxime | 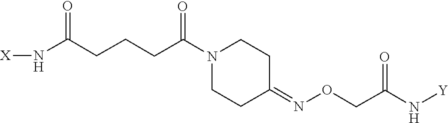 |
| BCY17901 | ALO | 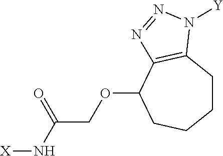 |

Y represents a bicycle ligand and X represents a modified oligonucleotide consisting of 21 linked nucleic acids conjugated at the 5′ end.

### Activity

The activity of RNAi compounds conjugated to CD71 bicycle ligands is tested in in heterozygous hTFRKI/+ knock in mice (described herein above).

### Treatment

hTFRKI/+ knock in mice are divided into groups of 2-4 mice each. Each mouse receives a single intravenous (i.v.) administration or subcutaneous (s.c.) administration of conjugated modified oligonucleotide or conjugated RNAi compound. A group of 4 mice receives PBS as a negative control.

### RNA Analysis

The mice are sacrificed one week post administration. RNA is extracted from various muscle tissues (including quadriceps (quad), gastrocnemius (gastroc), heart, liver) for quantitative real time RTPCR analysis to measure amount of mouse target RNA. Mouse target RNA levels are normalized to mouse GAPDH. Mouse GAPDH is amplified using mouse primer probe set mGapdh_LTS00102 (described herein above).

1. An oligomeric compound comprising a modified oligonucleotide and a conjugate group, wherein the modified oligonucleotide consists of 10 to 300 linked nucleosides, and the conjugate group comprises a bicycle ligand and a conjugate linker, wherein
the bicycle ligand comprises a polypeptide consisting of 13-22 linked amino acids or amino acid mimetics and a molecular scaffold, wherein
each of a first, a second, and a third amino acid of the polypeptide comprises a reactive group, each of which separately forms a bond with the molecular scaffold, thereby forming two polypeptide loops attached to the molecular scaffold; and
wherein a portion of the bicycle ligand binds to a type 1 transferrin receptor; and
wherein the modified oligonucleotide is covalently linked to the bicycle ligand through the conjugate linker.

2. The oligomeric compound of claim 1, wherein the conjugate group consists of the bicycle ligand and a conjugate linker.

3. The oligomeric compound of claim 1 or 2, wherein the oligomeric compound consists of the modified oligonucleotide and the conjugate group.

4. The oligomeric compound of any of claims 1-3, wherein the three reactive groups are each the thiol of a cysteine.

5. The oligomeric compound of any of claims 1-4, wherein the polypeptide has the following formula, from N-terminal to C-terminal:
[B]n-[Zᵢ]-[J]ₘ—[Zᵢᵢ]-[O]ₒ-[Zᵢᵢᵢ]—[U]ₚ, wherein:
Zᵢ, Zᵢᵢ, and Zᵢᵢᵢ are the first, second, and third amino acids comprising a reactive group;
each B, J, O, and U is independently selected amino acids or amino acid mimetics;
n is from 0 to 5;
m is from 3 to 7;
o is from 3 to 7;
p is from 0 to 5;
wherein the sum of m+o is less than 12.

6. The oligomeric compound of claim 5, wherein m is 7 and o is 3.

7. The oligomeric compound of claim 5, wherein m is 2 and o is 9.

8. The oligomeric compound of claim 5, wherein m and o are both 6.

9. The oligomeric compound of claim 5, wherein m is 3 and o is 8.

10. The oligomeric compound of any of claims 5-9, wherein n is 0.

11. The oligomeric compound of any of claims 5-9, wherein n is 3 or 4.

12. The oligomeric compound of any of claims 5-11, wherein p is 0.

13. The oligomeric compound of any of claims 5-11, wherein p is 3 or 4.

14. The oligomeric compound of any of claims 1-13, wherein the polypeptide has an N-terminal modification.

15. The oligomeric compound of claim 14, wherein the N-terminal modification is an acetyl group.

16. The oligomeric compound of claim 14, wherein the N-terminal modification is an azidopropyl group.

17. The oligomeric compound of any of claims 1-13, wherein the polypeptide has a C-terminal modification.

18. The oligomeric compound of claim 14, wherein the C-terminal modification is an amide group.

19. The oligomeric compound of any of claims 1-18, wherein the conjugate linker is attached to the N-terminal amino acid of the bicycle ligand.

20. The oligomeric compound of any of claims 1-19, wherein the conjugate linker is attached to the C-terminal amino acid of the bicycle ligand.

21. The oligomeric compound of any of claims 1-20, wherein the conjugate linker is attached to a side chain of an amino acid within one of the polypeptide loops of the bicycle ligand.

22. The oligomeric compound of any of claims 1-21, wherein the bicycle ligand comprises a C-terminal extension.

23. The oligomeric compound of claim 22, wherein the C-terminal extension is selected from PEG10 or PEG24.

24. The oligomeric compound of any of claims 1-23, wherein the bicycle ligand comprises an N-terminal extension.

25. The oligomeric compound of claim 24, wherein the N-terminal extension is selected from PEG10 or PEG24.

26. The oligomeric compound of any of claims 1-25, wherein the conjugate group is attached to the 5′-terminal nucleoside of the modified oligonucleotide.

27. The oligomeric compound of claim 26, wherein the conjugate group is attached to the 5′-position of the 5′-terminal nucleoside of the modified oligonucleotide.

28. The oligomeric compound of any of claims 1-25, wherein the conjugate group is attached to the 3′-terminal nucleoside of the modified oligonucleotide.

29. The oligomeric compound of claim 28, wherein the conjugate group is attached to the 3′-position of the 3′-terminal nucleoside of the modified oligonucleotide.

30. The oligomeric compound of any of claims 1-25, wherein the conjugate group is attached to an internal nucleoside of the modified oligonucleotide.

31. The oligomeric compound of claim 1-25, wherein the conjugate group is attached through a modified internucleoside linkage.

32. The oligomeric compound of claim 28-30, wherein the conjugate group is attached through a 2′-modified furanosyl sugar moiety.

33. The oligomeric compound of any of claims 1-32, wherein the bicycle ligand has an amino acid sequence with at least 80% identity to any of SEQ ID NO: 26-27, 36-56, 58-65, 67-76, 79-88, 90-152, or 192-246.

34. The oligomeric compound of claim 33, wherein the bicycle ligand has an amino acid sequence with at least 85%, at least 90%, at least 95%, or 100% identity to any of SEQ ID NO: 26-27, 36-56, 58-65, 67-76, 79-88, 90-152, or 192-246.

35. The oligomeric compound of any of claims 5-34, wherein [Zᵢ]-[J]ₘ—[Zᵢ]—[O]ₒ—[Zᵢᵢᵢ] has an amino acid sequence with at least 85%, at least 90%, at least 95%, or 100% identity to any of SEQ ID NO 26-27, 35-56, 58-65, 67-76, 79-88, 90-152, or 192-246.

36. The oligomeric compound of any of claims 5-35, wherein [Zᵢ]-[J]ₘ[Zᵢᵢ]—[O]ₒ—[Zᵢᵢᵢ] has an amino acid sequence of CXXDXXXGCISYC (SEQ ID NO: 35), wherein each “X” is an independently selected amino acid.

37. The oligomeric compound of any of claims 1-36, wherein the bicycle ligand comprises at least one, at least two, or at least three non-natural amino acids.

38. The oligomeric compound of claim 37, wherein at least one non-natural amino acid is selected from a _{D}-amino acid, allo-isoleucine, 2-amino-3-ethyl-pentanoic acid, aminoisobutyric acid, aminobutyric acid, azetidine, 7-azatryptophan, 6-azidolysine, β-cyclobutylalanine, β-methyl isoleucine, 4, 4-biphenylalanine, cis-hydroxyproline, cyclobutyl glycine, cyclohexyl glycine, cyclopentyl alanine, cyclopentyl glycine, 2,6-dimethyl tyrosine, 3,3-diphenyl alanine, 4-trans-hydroxy-L-proline, 1-napthaylalanine, 2-napthylalanine, N-methyl alanine, 1-methyl histidine, 3-methyl histidine, N-methyl-tryptophan, pipecolic acid,4-pyridylalanine, sarcosine, t-butyl alanine, or 3-t-butyl tyrosine.

39. The oligomeric compound of claim 38, wherein at least one non-natural amino acid is selected from 4-trans-hydroxy-L-proline, 6-azidolysine, and t-butyl glycine.

40. The oligomeric compound of any of claims 1-39, wherein the molecular scaffold is 1,1′,1″-(1,3,5-triazinane-1,3,5-triyl)triprop-2-en-1-one (TATA).

41. The oligomeric compound of any of claims 1-39, wherein the molecular scaffold is 1,1′,1″-(1,3,5-triazinane-1,3,5-triyl)tris(2-bromoethanone) (TATB).

42. The oligomeric compound of any of claims 1-41, wherein the bicycle ligand does not inhibit the binding of transferrin to the transferrin receptor.

43. The oligomeric compound of any of claims 1-42, wherein at least one nucleoside of the modified oligonucleotide comprises a modified sugar moiety.

44. The oligomeric compound of claim 43, wherein at least one modified sugar moiety comprises a bicyclic sugar moiety.

45. The oligomeric compound of claim 44, wherein the bicyclic sugar moiety comprises a 2′-4′ bridge selected from —O-CH2-; and —O—CH(CH3)-.

46. The oligomeric compound of claim 43-45, wherein at least one modified sugar moiety comprises a non-bicyclic modified sugar moiety.

47. The oligomeric compound of claim 46, wherein the non-bicyclic modified sugar moiety is a 2′-MOE sugar moiety or 2′-OMe sugar moiety.

48. The oligomeric compound of any of claims 43-47, wherein at least one nucleoside of the modified oligonucleotide compound comprises a sugar surrogate.

49. The oligomeric compound of any of claims 1-48, wherein the modified oligonucleotide comprises at least one modified internucleoside linkage.

50. The oligomeric compound of claim 49, wherein at least one modified internucleoside linkage is a phosphorothioate internucleoside linkage.

51. The oligomeric compound of any of claims 49-50, wherein each internucleoside linkage is a modified internucleoside linkage.

52. The oligomeric compound of claim 51, wherein each internucleoside linkage is a phosphorothioate internucleoside linkage.

53. The oligomeric compound of any of claims 1-50, wherein the modified oligonucleotide comprises at least one phosphodiester internucleoside linkage.

54. The oligomeric compound of any of claims 1-50 or 53, wherein each internucleoside linkage of the modified oligonucleotide is independently selected from a phosphodiester or a phosphorothioate internucleoside linkage.

55. The oligomeric compound of claim 49, wherein at least one modified internucleoside linkage is a mesyl phosphoramidate internucleoside linkage.

56. The oligomeric compound of any of claims 49-55, wherein each internucleoside linkage is independently selected from a phosphodiester, a phosphorothioate internucleoside, or a mesyl phosphoramidate internucleoside linkage.

57. The oligomeric compound of any of claims 49-55, wherein each internucleoside linkage is independently selected from a phosphorothioate internucleoside or a mesyl phosphoramidate internucleoside linkage.

58. The oligomeric compound of any of claims 1-57, wherein the modified oligonucleotide comprises at least one modified nucleobase.

59. The oligomeric compound of claim 58, wherein the modified nucleobase is 5-methylcytosine.

60. The oligomeric compound of any of claims 1-59, wherein the modified oligonucleotide comprises a deoxy region consisting of 5-12 contiguous 2′-deoxynucleosides.

61. The oligomeric compound of claim 60, wherein each nucleoside of the deoxy region is a 2′-β-D-deoxynucleoside.

62. The oligomeric compound of claim 60 or 61, wherein the deoxy region consists of 7, 8, 9, 10, or 7-10 linked nucleosides.

63. The oligomeric compound of any of claims 60-62, wherein each nucleoside immediately adjacent to the deoxy region comprises a modified sugar moiety.

64. The oligomeric compound of any of claims 60-63, wherein the deoxy region is flanked on the 5′-side by a 5′-region consisting of 1-6 linked 5′-region nucleosides and on the 3′-side by a 3′-region consisting of 1-6 linked 3′-region nucleosides; wherein
the 3′-most nucleoside of the 5′-region comprises a modified sugar moiety; and
the 5′-most nucleoside of the 3′-region comprises a modified sugar moiety.

65. The oligomeric compound of claim 64, wherein each nucleoside of the 3′-region comprises a modified sugar moiety.

66. The oligomeric compound of claim 64 or 65, wherein each nucleoside of the 5′-region comprises a modified sugar moiety.

67. The oligomeric compound of any of claims 1-66, wherein the modified oligonucleotide has a sugar motif comprising:
a 5′-region consisting of 1-6 linked 5′-region nucleosides;
an internal region consisting of 6-10 linked internal region nucleosides; and
a 3′-region consisting of 1-6 linked 3′-region nucleosides;
wherein each of the 5′-region nucleosides and each of the 3′-region nucleosides comprises a modified sugar moiety; and each of the internal region nucleosides is selected from a 2′-deoxynucleoside and a 2′-substituted nucleoside.

68. The oligomeric compound of claim 67, wherein the modified oligonucleotide has a sugar motif comprising:
a 5′-region consisting of 1-6 linked 5′-region nucleosides;
an internal region consisting of 6-10 linked internal region nucleosides; and
a 3′-region consisting of 1-6 linked 3′-region nucleosides;
wherein each of the 5′-region nucleosides and each of the 3′-region nucleosides is a cEt nucleoside or a 2′-MOE nucleoside; and each of the internal region nucleosides is a 2′-β-D-deoxynucleoside.

69. The oligomeric compound of any of claims 1-59, wherein each nucleoside of the modified oligonucleotide comprises a 2′-sugar moiety.

70. The oligomeric compound of claim 69, wherein each 2′-sugar moiety is selected from 2′-OMe, 2′-MOE, or 2′-NMA.

71. The oligomeric compound of claim 69 or 70, wherein each nucleoside of the modified oligonucleotide comprises the same 2′-sugar moiety.

72. The oligomeric compound of any of claims 1-71, wherein the conjugate linker is cleavable.

73. The oligomeric compound of any of claims 1-72, wherein the conjugate linker comprises 1-3 linker nucleosides.

74. The oligomeric compound of any of claims 1-72, wherein the conjugate linker does not comprise any linker nucleosides.

75. The oligomeric compound of any of claims 1-74, wherein the conjugate group comprises:

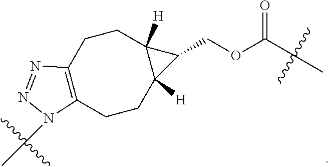 [SMILES: *C(=O)OC[C@@H]1[C@@H]2CCc3c(nnn3*)CC[C@@H]21]

76. The oligomeric compound of any of claims 1-74, wherein the conjugate group comprises:

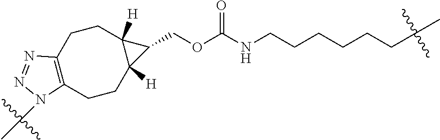 [SMILES: *CCCCCCNC(=O)OC[C@@H]1[C@@H]2CCc3c(nnn3*)CC[C@@H]21]

77. The oligomeric compound of any of claims 1-74, wherein the conjugate group comprises:

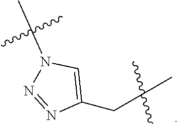 [SMILES: *Cc1cn(*)nn1]

78. The oligomeric compound of any of claims 1-74, wherein the conjugate group comprises: 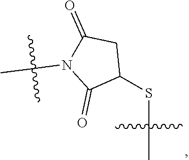 [SMILES: *SC1CC(=O)N(*)C1=O]
optionally

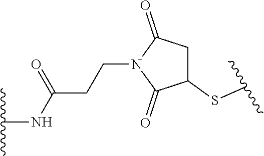 [SMILES: *NC(=O)CCN1C(=O)CC(S*)C1=O]

79. The oligomeric compound of any of claims 1-74, wherein the conjugate group comprises:

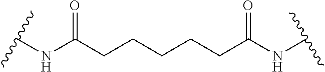

80. The oligomeric compound of any of claims 1-74, wherein the conjugate group comprises:

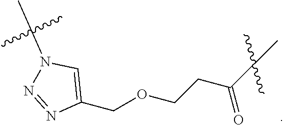 [SMILES: *C(=O)CCOCc1cn(*)nn1]

81. The oligomeric compound of any of claims 1-74, wherein the conjugate group comprises:

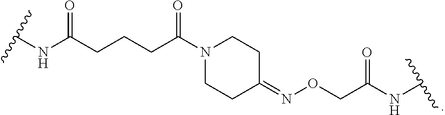 [SMILES: *NC(=O)CCCC(=O)N1CCC(=NOCC(=O)N*)CC1]

82. The oligomeric compound of any of claims 1-74, wherein the conjugate group comprises:

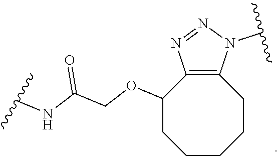 [SMILES: *NC(=O)COC1CCCCCc2c1nnn2*]

83. The oligomeric compound of any of claims 1-82, wherein the modified oligonucleotide is complementary to a target nucleic acid expressed in muscle.

84. The oligomeric compound of any of claims 1-83, wherein the modified oligonucleotide is capable of reducing the amount of a target nucleic acid through the activation of Rnase H.

85. The oligomeric compound of any of claims 1-83, wherein the modified oligonucleotide is capable of reducing the amount of a target nucleic acid through the activation of RISC/Ago2.

86. The oligomeric compound of any of claims 1-83, wherein the modified oligonucleotide is capable of modulating the splicing of a target nucleic acid.

87. The oligomeric compound of any of claims 1-83, wherein the modified oligonucleotide is a guide RNA, a tracrRNA, or a scout RNA.

88. The oligomeric compound of any of claims 1-87, wherein the modified oligonucleotide is complementary to the complement of a target nucleic acid expressed in muscle.

89. The oligomeric compound of any of claims 83-88, wherein the target nucleic acid is associated with a muscle disease.

90. The oligomeric compound of any of claims 83-89, wherein the target nucleic acid is selected from CaMK2d, NLRP3, PLN, DMD, DMPK, DNM2, DUX4, or HPRT.

91. The oligomeric compound of any of claims 83-90, wherein the muscle target nucleic acid has a sequence selected from any of SEQ ID NO: 1-15.

92. The oligomeric compound of any of claims 83-91, wherein the target nucleic acid is expressed in at least one of expressed in at least one of the following tissues: skeletal muscle (including but not limited to quadriceps, gastrocnemius, tibialis anterior, triceps, masseter, extensor digitorum longus (EDL), soleus, diaphragm), heart, sciatic nerve, aorta, or liver.

93. The oligomeric compound of any of claims 1-92, wherein the nucleobase sequence of the modified oligonucleotide comprises at least 12, at least 13, at least 14, at least 15, or at least 16 contiguous nucleobases of any of the nucleobase sequences of any of SEQ ID NO: 167-191.

94. The oligomeric compound of any of claims 1-93, wherein the modified oligonucleotide consists of 10 to 25, 10 to 30, 12 to 20, 12 to 25, 12 to 30, 13 to 20, 13 to 25, 13 to 30, 14 to 20, 14 to 25, 14 to 30, 15 to 20, 15 to 25, 15 to 30, 16 to 18, 16 to 20, 16 to 25, 16 to 30, 17 to 20, 17 to 25, 17 to 30, 18 to 20, 18 to 25, 18 to 30, 19 to 20, 19 to 25, 19 to 30, 20 to 25, 20 to 30, 21 to 25, 21 to 30, 21 to 50, 22 to 25, 22 to 30, 23 to 25, 23 to 30, 20 to 100, 40 to 100, 50 to 100, 50 to 200, 100 to 300, 150-300, or 200-300 linked nucleosides.

95. An oligomeric duplex, comprising a first oligomeric compound comprising a first modified oligonucleotide and a second compound oligomeric comprising a second modified oligonucleotide consisting of 16 to 30 linked nucleosides, wherein the nucleobase sequence of the second modified oligonucleotide comprises a complementary region of at least 12 nucleobases that is at least 90% complementary to an equal length portion of the first modified oligonucleotide, and wherein the second oligomeric compounds is an oligomeric compound of any of claims 1-86 or 89-94.

96. The oligomeric duplex of claim 95, wherein the first modified oligonucleotide is complementary to a muscle target nucleic acid.

97. The oligomeric duplex of claim 96, wherein the duplex is capable of reducing the amount of a target nucleic acid through the activation of RISC/Ago2.

98. The oligomeric duplex of any of claims 95-97, wherein at least one nucleoside of the second modified oligonucleotide comprises a modified sugar moiety.

99. The oligomeric duplex of claim 98, wherein the modified sugar moiety of the second modified oligonucleotide comprises a bicyclic sugar moiety.

100. The oligomeric duplex of claim 99, wherein the bicyclic sugar moiety of the second modified oligonucleotide comprises a 2′-4′ bridge selected from —O—CH₂—; and —O—CH(CH₃)—.

101. The oligomeric duplex of claim 100, wherein the modified sugar moiety of the second modified oligonucleotide comprises a non-bicyclic modified sugar moiety.

102. The oligomeric duplex of claim 101, wherein the non-bicyclic modified sugar moiety of the second modified oligonucleotide is a 2′-MOE sugar moiety, a 2′-F sugar moiety, or 2′-OMe sugar moiety.

103. The oligomeric duplex of any of claims 95-102, wherein at least one nucleoside of the second modified oligonucleotide comprises a sugar surrogate.

104. The oligomeric duplex of any of claims 95-102, wherein at least one internucleoside linkage of the second modified oligonucleotide is a modified internucleoside linkage.

105. The oligomeric duplex of claim 104, wherein at least one modified internucleoside linkage of the second modified oligonucleotide is a phosphorothioate internucleoside linkage.

106. The oligomeric duplex of any of claims 95-105, wherein at least one internucleoside linkage of the second modified oligonucleotide is a phosphodiester internucleoside linkage.

107. The oligomeric duplex of any of claims 95-104 or 106, wherein each internucleoside linkage of the second modified oligonucleotide is independently selected from a phosphodiester or a phosphorothioate internucleoside linkage.

108. The oligomeric duplex of any of claims 95-107, wherein the second modified oligonucleotide comprises at least one modified nucleobase.

109. The oligomeric duplex of claim 108, wherein the modified nucleobase of the second modified oligonucleotide is 5-methylcytosine.

110. The oligomeric duplex of any of claims 95-109, wherein the first oligomeric compound comprises a 5′-stabilized phosphate group.

111. The oligomeric duplex of any of claims 95-109, wherein the second oligomeric compound comprises a 5′-stabilized phosphate group.

112. The oligomeric duplex of claim 110 or 111, wherein the stabilized phosphate group comprises a cyclopropyl phosphonate or a vinyl phosphonate.

113. The oligomeric duplex of any of claims 95-112, wherein the first modified oligonucleotide of comprises a glycol nucleic acid (GNA) sugar surrogate.

114. The oligomeric duplex of any of claims 95-113, wherein first modified oligonucleotide comprises a 2′-NMA sugar moiety.

115. The oligomeric duplex of any of claims 95-114, wherein the second modified oligonucleotide comprises a glycol nucleic acid (GNA) sugar surrogate.

116. The oligomeric duplex of any of claims 95-115, wherein the second modified oligonucleotide compound comprises a 2′-NMA sugar moiety.

117. A method of modulating a nucleic acid target in a subject, comprising administering to the subject an oligomeric compound according to any of claims 1-94 or an oligomeric duplex of any of claims 95-116.

118. The method of claim 117, wherein the nucleic acid target is expressed in at least one of skeletal muscle (including but not limited to quadriceps, gastrocnemius, tibialis anterior, triceps, masseter, extensor digitorum longus (EDL), soleus, diaphragm), heart, sciatic nerve, aorta, or liver.

119. The method of claim 117 or 118, wherein administration of the oligomeric compound of any of claims 1-94 or the oligomeric duplex of any of claims 95-116 results in reduction of the nucleic acid target.

120. The method of claim 117 or 118, wherein administration of the oligomeric compound of any of claims 1-94 or the oligomeric duplex of any of claims 95-116 results in a change in the splicing of the nucleic acid target.

121. The method of any of claims 117-120, wherein the oligomeric compound or oligomeric duplex is administered via intravenous or subcutaneous dosing.

122. The method of any of claims 117-120, wherein the oligomeric compound or oligomeric duplex is administered at a dose of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, or 350 mg.

123. The method of any of claims 117-122, comprising administering the oligomeric compound or oligomeric duplex once every 4 weeks, once every 6 weeks, once every 8 weeks, once every 12 weeks, once every 16 weeks, once every 20 weeks, once every 24 weeks, once every 6 months, or once a year.

124. A bicycle ligand specific for transferrin receptor 1 (TfR1) which comprises an amino acid sequence selected from: CP[HyP]DAYLGC[tBuGly]SYCEPWK (SEQ ID NO: 245, herein referred to as BCY21757) and CP[HyP]DAYLGC[tBuGly]SYCEPWC (SEQ ID NO: 246, herein referred to as BCY21758), wherein HyP represents trans-4-hydroxy-L-proline and tBuGly represents t-butyl-glycine.

125. The bicycle ligand of claim 124, which comprises an N-terminal acetyl group and a C-terminal CONH₂ group.

126. The bicycle ligand of claim 124 or 125, which is a pharmaceutically acceptable salt.

127. The pharmaceutical salt of claim 126, wherein the pharmaceutically acceptable salt is selected from the sodium, potassium, calcium or ammonium salt.

128. The bicycle ligand of claim 124, wherein the first, second and third cysteine residues within said peptide ligands are covalently bonded to a molecular scaffold such that two polypeptide loops are formed on said molecular scaffold.

129. The bicycle ligand of claim 125, wherein the molecular scaffold is 1,1′,1″-(1,3,5-triazinane-1,3,5-triyl)triprop-2-en-1-one (TATA).
